SEQUENCE LISTING (48 sequences)

SEQ ID NO: 1 - DNA, 30 bp, Artificial Sequence
  Portion of non-productive TCR gene (out-of-frame prevents expression)
gtgccagcagcgaaaggggtcgagcagtat

SEQ ID NO: 2 - protein, 7 aa, Artificial Sequence
  Portion of non-productive TCR gene (out-of-frame prevents expression)
CASSEQY

SEQ ID NO: 3 - DNA, 30 bp, Artificial Sequence
  Portion of non-productive TCR gene (stop codon prevents expression)
gtgcaagtcgctagagggaactccggaagc

SEQ ID NO: 4 - protein, 10 aa, Artificial Sequence
  Portion of non-productive TCR gene (stop codon prevents expression)
CASRRETPEA

SEQ ID NO: 5 - DNA, 25 bp, Artificial Sequence
  Portion of non-productive TCR gene (2nd V(D)J recombination event)
gtgccagccggacctcccacggcta

SEQ ID NO: 6 - protein, 9 aa, Artificial Sequence
  Portion of non-productive TCR gene (2nd V(D)J recombination event)
CASRTSHGY

SEQ ID NO: 7 - DNA, 27 bp, Artificial Sequence
  Portion of repaired TCR found in peripheral blood (mutate T to C)
tgtgccagcagtgacgggactcagtac

SEQ ID NO: 8 - DNA, 29 bp, Artificial Sequence
  Portion of repaired TCR gene (delete C)
gtgccagcaggaaaggggtcgagcagtat

SEQ ID NO: 9 - protein, 10 aa, Artificial Sequence
  Portion of repaired TCR gene (delete C)
CASRKGVEQY

SEQ ID NO: 10 - DNA, 29 bp, Artificial Sequence
  Portion of repaired TCR gene (delete G)
gtgccagcagcaaaggggtcgagcagtat

SEQ ID NO: 11 - protein, 10 aa, Artificial Sequence
  Portion of repaired TCR gene (delete G)
CASSKGVEQY

SEQ ID NO: 12 - DNA, 29 bp, Artificial Sequence
  Portion of repaired TCR gene (delete A)
gtgccagcagcgaaggggtcgagcagtat

SEQ ID NO: 13 - protein, 10 aa, Artificial Sequence
  Portion of repaired TCR gene (delete A)
CASSEGVEQY

SEQ ID NO: 14 - DNA, 29 bp, Artificial Sequence
  Portion of repaired TCR gene (delete A)
gtgccagcagcgaaggggtcgagcagtat

SEQ ID NO: 15 - protein, 9 aa, Artificial Sequence
  Portion of repaired TCR found in peripheral blood (mutate T to C)
CASSDGTQY

SEQ ID NO: 16 - DNA, 29 bp, Artificial Sequence
  Portion of repaired TCR gene (delete T)
gtgccagcagcgaaaggggcgagcagtat

SEQ ID NO: 17 - protein, 10 aa, Artificial Sequence
  Portion of repaired TCR gene (delete T)
CASSERGEQY

SEQ ID NO: 18 - DNA, 34 bp, Artificial Sequence
  Portion of non-productive TCR gene (stop codon prevents expression)
gtgcaagtcgctagagggaaactccggaagcttt

SEQ ID NO: 19 - protein, 11 aa, Artificial Sequence
  Portion of non-productive TCR gene (stop codon prevents expression)
CASRRETPEAF

SEQ ID NO: 20 - DNA, 34 bp, Artificial Sequence
  Portion of repaired TCR gene (mutate A to C)
gtgcaagtcgctcgagggaaactccggaagcttt

SEQ ID NO: 21 - protein, 12 aa, Artificial Sequence
  Portion of repaired TCR gene (mutate A to C)
CASRSRETPEAF

SEQ ID NO: 22 - DNA, 34 bp, Artificial Sequence
  Portion of repaired TCR gene (mutate A to G)
gtgcaagtcgctggagggaaactccggaagcttt

SEQ ID NO: 23 - protein, 12 aa, Artificial Sequence
  Portion of repaired TCR gene (mutate A to G)
CASRWRETPEAF

SEQ ID NO: 24 - DNA, 34 bp, Artificial Sequence
  Portion of repaired TCR gene (mutate A to T)
gtgcaagtcgcttgagggaaactccggaagcttt

SEQ ID NO: 25 - protein, 12 aa, Artificial Sequence
  Portion of repaired TCR gene (mutate A to T)
CASRLRETPEAF

SEQ ID NO: 26 - DNA, 36 bp, Artificial Sequence
  Portion of donor TCR gene
tgtgccagctctgagggaggccaagacacccagtac

SEQ ID NO: 27 - protein, 6 aa, Artificial Sequence
  Portion of donor TCR gene
SEGGQD

SEQ ID NO: 28 - DNA, 33 bp, Artificial Sequence
  Portion of convergent V(D)J recombination
tgtgccagcagtcgacagggtggtgaacagtac

SEQ ID NO: 29 - protein, 5 aa, Artificial Sequence
  Portion of convergent V(D)J recombination
SRQGG

SEQ ID NO: 30 - DNA, 33 bp, Artificial Sequence
  Portion of recipient TCR gene
tgtgccagcagccgacagggagggaaacagtac

SEQ ID NO: 31 - DNA, 27 bp, Artificial Sequence
  Portion of repaired TCR found in peripheral blood (mutate T to G)
tgtgccagcagtgacgggactgagtac

SEQ ID NO: 32 - DNA, 27 bp, Artificial Sequence
  Portion of productive TCR gene found in peripheral blood
tgtgccagcaggggggaggttgacacc

SEQ ID NO: 33 - protein, 9 aa, Artificial Sequence
  Portion of productive TCR gene found in peripheral blood
CASRGEVDT

SEQ ID NO: 34 - DNA, 25 bp, Artificial Sequence
  Portion of non-productive TCR found in peripheral blood with out-of-frame V and J gene segments
tgtgccaccgtatggagggaagtac

SEQ ID NO: 35 - DNA, 24 bp, Artificial Sequence
  Portion of repaired TCR found in peripheral blood (delete G)
tgtgccacctatggagggaagtac

SEQ ID NO: 36 - protein, 8 aa, Artificial Sequence
  Portion of repaired TCR found in peripheral blood (delete G)
CATYGGKY

SEQ ID NO: 37 - DNA, 24 bp, Artificial Sequence
  Portion of repaired TCR found in peripheral blood (delete T)
tgtgccaccgatggagggaagtac

SEQ ID NO: 38 - protein, 8 aa, Artificial Sequence
  Portion of repaired TCR found in peripheral blood (delete T)
CATDGGKY

SEQ ID NO: 39 - DNA, 24 bp, Artificial Sequence
  Portion of repaired TCR found in peripheral blood (delete A)
tgtgccaccgttggagggaagtac

SEQ ID NO: 40 - protein, 8 aa, Artificial Sequence
  Portion of repaired TCR found in peripheral blood (delete A or T)
CATVGGKY

SEQ ID NO: 41 - DNA, 24 bp, Artificial Sequence
  Portion of repaired TCR found in peripheral blood (delete T)
tgtgccaccgtaggagggaagtac

SEQ ID NO: 42 - protein, 9 aa, Artificial Sequence
  Portion of repaired TCR found in peripheral blood (mutate T to G)
CASSDGTEY

SEQ ID NO: 43 - DNA, 24 bp, Artificial Sequence
  Portion of repaired TCR found in peripheral blood (delete G)
tgtgccaccgtatggaggaagtac

SEQ ID NO: 44 - protein, 8 aa, Artificial Sequence
  Portion of repaired TCR found in peripheral blood (delete G)
CATVWREY

SEQ ID NO: 45 - DNA, 27 bp, Artificial Sequence
  Portion of non-productive TCR found in peripheral blood encoding a stop codon in a somatic junction
tgtgccagcagtgacgggacttagtac

SEQ ID NO: 46 - protein, 8 aa, Artificial Sequence
  Portion of non-productive TCR found in peripheral blood encoding a stop codon in a somatic junction
CASSDGTY

SEQ ID NO: 47 - DNA, 27 bp, Artificial Sequence
  Portion of repaired TCR found in peripheral blood (mutate T to A)
tgtgccagcagtgacgggactaagtac

SEQ ID NO: 48 - protein, 9 aa, Artificial Sequence
  Portion of repaired TCR found in peripheral blood (mutate T to A)
CASSDGTKY

(19) United States
(12) Patent Application Publication
(10) Pub. No.: US 2024/0161874 A1
(43) Pub. Date: May 16, 2024

(54) METHODS FOR RECONSTITUTING T CELL SELECTION AND USES THEREOF

(71) Applicant: THE BOARD OF REGENTS OF THE UNIVERSITY OF TEXAS SYSTEM, Austin, TX (US)

(72) Inventors: Scott CHRISTLEY, Austin, TX (US); Benjamin GREENBERG, Austin, TX (US); Linsay COWELL, Austin, TX (US); Jared OSTMEYER, Austin, TX (US)

(73) Assignee: THE BOARD OF REGENTS OF THE UNIVERSITY OF TEXAS SYSTEM, Austin, TX (US)

(21) Appl. No.: 18/281,085

(22) PCT Filed: Mar. 11, 2022
(86) PCT No.: PCT/US2022/019992

§ 371 Date: Sep. 8, 2023

(51) Int. Cl. G16B 40/20; G16H 50/30

(52) U.S. Cl. CPC G16B 40/20; G16H 50/30

(57) ABSTRACT

Provided herein is a machine learning model to reconstitute T cell and B cell selections, and methods of use thereof. The methods provided herein include methods of prediction of the risk of developing an autoimmune disease or disorder, the risk of developing alloimmunity from organ or cellular transplant, the risk of developing graft-versus-host disease (GvHD) from organ or cellular transplant, the risk of developing alloimmunity from an adoptive T cell therapy, the risk of developing alloimmunity from a chimeric antigen receptor (CAR)-T cell therapy, and methods of prediction of the safety of an antibody drug in a subject. Also provided herein is a method of classifying T cell receptor p (TCRp) gene, and methods of use thereof. The methods provided include methods of determining an organ donor/organ recipient compatibility, methods of predicting graft versus host disease (GvHD) in a recipient, acute GvHD (aGvHD), chronic GvHD (cGvHD) and cancer relapse in a subject.

### CROSS-REFERENCE TO RELATED APPLICATIONS

This application claims benefit of priority under 35 U.S.C. § 119(e) of U.S. Provisional Applications Nos. 63/160,299, filed Mar. 12, 2021, and 63/274,263, filed Nov. 1, 2021. The disclosure of the prior applications is considered part of and is herein incorporated by reference in the disclosure of this application in its entirety.

### INCORPORATION OF SEQUENCE LISTING

The material in the accompanying sequence listing is hereby incorporated by reference into this application. The accompanying sequence listing text file, name 426871-000252_SL_ST25.txt, was created on Mar. 9, 2022, and is 17 kb. The file can be assessed using Microsoft Word on a computer that uses Windows OS.

### BACKGROUND

T cells are one of the most important cells of the human immune system and play a central role the body's adaptive immune response. T-cell receptors (TCRs) are protein sequences found on the surface of T cells that dictate which antigens the T cell can bind to and interact with. TCR genes are created without regard for which antigens the TCR can bind, making it essential that developing T cells undergo T cell selection in order to build immune tolerance. For example, it is important that some TCRs are culled during T cell selection to prevent development of T cells that might attack healthy tissues. Humans naturally provide a huge variety of TCRs through the mutation of TCR genes during T cell development. This diversity in TCR genes is important factor for a healthy immune system ensuring the body's immune system can respond to a variety of different antigens. However, the large volume of TCR genes produced during T cell development makes simulating T cell selection difficult using conventional tools. To generate more accurate and personalized models of T cell selection, it is desirable to develop machine learning systems that can predict whether TCRs would or would not survive T cell selection. It is also desirable to use the machine learning systems to predict other cell selection processes (e.g., B cell selection) and use the predictions in a variety of clinical applications.

### SUMMARY

The sequences of an immune cell receptor dictate if an immune cell passes or fails immune cell selection (e.g., T cell selection, B cell selection, and the like). There is an unmet need for methods of predicting if an immune cell passes or fails immune cell selection. Such methods implemented for example in a machine learning system will have substantial applications in the immunological field in general, and in the autoimmunity, alloimmunity and onco-immunology fields in particular.

An embodiment provides a method of classifying an immune receptor chain gene comprising: a) obtaining an immune receptor chain gene sequence comprising multiple gene segments and somatic alterations; b) translating at least one of the multiple gene segments or somatic alterations into an amino acid sequence; c) identifying an immune receptor chain gene encoding an amino acid sequence capable of antigen recognition as a productive immune receptor chain, d) identifying an immune receptor chain gene without an amino acid sequence capable of antigen recognition as a non-productive immune receptor chain gene, e) repairing an immune receptor chain gene identified as non-productive to generate a repaired immune receptor chain gene, having an amino acid sequence capable of antigen recognition, and f) classifying the immune receptor chain gene as a productive immune receptor chain gene or as a repaired immune receptor chain gene, thereby classifying the immune receptor chain gene.

The gene segments can be selected from the group consisting of variable (V) gene segments, diversity (D) gene segments, joining (J) gene segments, and any combination thereof. The immune receptor chain gene can be selected from the group consisting of T cell receptor (TCR), TCR alpha chain (TCRα), TCR beta chain (TCRβ), TCR delta chain (TCRβ), TCR gamma chain (TCRγ), B cell receptor (BCR), BCR light chain (BCRL), BCR heavy chain (BCRH), immunoglobulin light chain (IgL), immunoglobulin heavy chain (IgH), immunoglobulin kappa chain (Igκ) and immunoglobulin lambda chain (Igλ). For example, the immune receptor chain gene can be a TCRβ gene. The non-productive TCRβ gene can be a TCRβ gene with out-of-frame gene segments or a TCRβ gene with a stop codon in a somatic junction between gene segments. Repairing non-productive TCRβ gene can comprise adding or removing one or more nucleotides at a somatic junction between gene segments to bring the gene segments in a same reading frame and/or mutating a nucleotide in a somatic region between gene segments to convert a stop codon into an amino acid. The TCRβ gene sequence can comprise a complimentary determining region 1 (CDR1) sequence of the TCRβ gene, a CDR2 sequence of the TCRβ gene, a CDR3 sequence of the TCRβ gene, a combination thereof, or a sequence of a complete TCRβ gene. For example, the TCRβ gene sequence can be a CDR3 sequence of the TCRβ gene. Further the first three amino acids and the last three amino acids of the CDR3 sequences can be removed from the TCRβ gene sequence. Obtaining a TCRβ gene sequence can comprise sequencing TCRβ genes in a blood sample from a subject. The blood sample can be a peripheral blood mononucleated cell sample. Obtaining a TCRβ gene sequence can comprise further isolating T cells from a sample. Isolating T cells can be by cell sorting and/or RNA expression. T cells can be non-regulatory T cells. The subject can be human.

Another embodiment provides a method of determining an organ donor/organ recipient compatibility comprising: a) classifying T cell receptor β (TCRβ) genes of the organ donor and TCRβ genes of the organ recipient as productive TCRβ gene or repaired TCRβ gene using the method described herein; b) comparing a number of productive and repaired TCRβ genes in a donor to a number of productive TCRβ genes in a recipient; and c) quantifying the fraction of TCRβ from the organ recipient that are compatible with the organ donor, thereby determining an organ donor/organ recipient compatibility.

Quantifying can comprise calculating a post selection fraction PSF score. A PSF score can be a ratio between the number of compatible TCRβ genes from the organ recipient and the total number of TCRβ genes. The PSF score can range from 0 to 1. The PSF score can be a PSF_{RECIPIENT} score, wherein the PSF_{RECIPIENT} score is a ratio between F_{PROD} and F_{TOTAL}, wherein F_{TOTAL} is F_{REPAIR}+F_{PROD}, and wherein F_{PROD} is a number of TCRβ genes identified as productive TCRβ genes in both the organ recipient and the organ donor, and F_{REPAIR} is a number of TCRβ genes identified as repaired TCRβ genes in the organ donor and identified as productive TCRβ genes in the organ recipient. A PSF_{RECIPIENT} of zero can indicate that none the TCRβ genes sequenced in the organ recipient are compatible with the organ donor. A PSF_{RECIPIENT} score of 1 can indicate that all the TCRβ genes sequenced in the organ recipient are compatible with the organ donor. Where the PSF_{RECIPIENT} score is not favorable, the organ transplant may not go forward. Where the PSF_{RECIPIENT} score is favorable the organ donor's organ can be transplanted into the recipient. The TCRβ gene sequence can comprise a CDR3 sequence of the TCRβ gene. The first three amino acids and the last three amino acids of the CDR3 sequences from the TCRβ gene sequence can be removed.

An additional embodiment provides a method of predicting graft versus host disease (GvHD) in a recipient comprising: a) classifying T cell receptor β (TCRβ) genes of the donor and TCRβ genes of the recipient as productive TCRβ gene or repaired TCRβ gene using the method described herein; b) comparing a number of productive and repaired TCRβ genes in the recipient to a number of productive TCRβ genes in the donor; and c) quantifying the fraction of TCRβ from the donor that are compatible with the recipient, thereby predicting GvHD in a recipient.

The GvHD can be acute GvHD (aGvHD). The organ or cells can be bone marrow or a hematopoietic stem cell transplant. Predicting aGvHD can comprise quantifying a number of productive TCRβ genes from the donor that are compatible with the recipient. Quantifying can comprise calculating a post selection fraction PSF_{DONOR-PROD} score, wherein the PSF_{DONOR-PROD} score is a ratio between F_{PROD} and F_{TOTAL}, wherein F_{TOTAL} is F_{REPAIR}+F_{PROD}, and wherein F_{PROD} is a number of TCRβ genes identified as productive TCRβ genes in both the donor and the recipient, and F_{REPAIR} is a number of TCRβ genes identified as repaired TCRβ genes in the recipient and identified as productive TCRβ genes in the donor. The PSF_{DONOR-PROD} can range from 0 to 1. A PSF_{DONOR-PROD} of zero can indicate that none the TCRβ genes sequenced in the donor are compatible with the recipient. A PSF_{DONOR-PROD} score of 1 can indicate that all the TCRβ genes sequenced in the donor are compatible with the recipient. Where the PSF_{DONOR-PROD} score is unfavorable the organ or cellular transplant may not go forward. Where the PSF_{DONOR-PROD} score is favorable the donor's organ or cells can be transplanted into the recipient. The TCRβ gene sequence can comprise a CDR3 sequence of the TCRβ gene. The first three amino acids and the last three amino acids of the CDR3 sequences from the TCRβ gene sequence can be removed.

The GvHD can be chronic GvHD (cGvHD). The organ or cells can be bone marrow or a hematopoietic stem cell transplant. Predicting cGvHD can comprise quantifying a number of repaired TCRβ gene from the donor that are compatible with the recipient. Quantifying can comprise calculating a post selection fraction PSF_{DONOR-REPAIR} score, wherein the PSF_{DONOR-REPAIR} score is a ratio between F_{PROD} and F_{TOTAL}, wherein F_{TOTAL} is F_{REPAIR}+F_{PROD}, and wherein F_{PROD} is a number of TCRβ genes identified as productive TCRβ genes in the recipient and identified as repaired in the donor, and F_{REPAIR} is a number of TCRβ genes identified as repaired TCRβ genes in both the recipient and the donor. The PSF_{DONOR-REPAIR} can range from 0 to 1. A PSF_{DONOR-REPAIR} of zero can indicate that none the TCRβ genes sequenced in the donor are compatible with the recipient. A PSF_{DONOR-REPAIR} score of 1 can indicate that all the TCRβ genes sequenced in the donor are compatible with the recipient. Where the PSF_{DONOR-REPAIR} score is unfavorable the organ or cellular transplant may not go forward. Where the PSF_{DONOR-REPAIR} score is favorable the donor's organ or cells can be transplanted into the recipient. The TCRβ gene sequence can comprise a CDR3 sequence of the TCRβ gene. The first three amino acids and the last three amino acids of the CDR3 sequences from the TCRβ gene sequence can be removed.

An embodiment provides a method of predicting cancer relapse in a hematopoietic stem cell recipient comprising: a) classifying T cell receptor β (TCRβ) genes of a hematopoietic stem cell donor and TCRβ genes of a hematopoietic stem cell recipient as productive TCRβ gene or repaired TCRβ gene using the method described here; b) comparing a number of repaired TCRβ genes in both the hematopoietic stem cell donor and the hematopoietic stem cell recipient; and c) quantifying a number of repaired TCRβ genes in the hematopoietic stem cell donor that are not found in the hematopoietic stem cell recipient, thereby predicting cancer relapse in the hematopoietic stem cell recipient.

The hematopoietic stem cell recipient can be a subject having cancer. Repaired TCRβ genes from the hematopoietic stem cell donor that are absent in the hematopoietic stem cell recipient can be likely to produce a T cell receptor (TCR) that recognizes cancer cells in the hematopoietic stem cell recipient. Quantifying can comprise calculating a (NOVEL score, wherein the f_{NOVEL} score is the fraction of the total number of TCRβ genes identified as repaired TCRβ genes in the hematopoietic stem cell donor excluding the number of repaired TCRβ genes that are in common between the hematopoietic stem cell recipient and the hematopoietic stem cell donor. The lower the f_{NOVEL} score between the hematopoietic stem cell recipient and the hematopoietic stem cell donor is, the higher the risk of cancer relapse can be. The higher the f_{NOVEL} score between the hematopoietic stem cell recipient and the hematopoietic stem cell donor is, the higher the chance of an absence of cancer relapse can be. Where the f_{NOVEL} score is unfavorable the organ or cellular transplant may not go forward. Where the f_{NOVEL} score is favorable the donor's organ or cells can be transplanted into the recipient. The TCRβ gene sequence can comprise a CDR3 sequence of the TCRβ gene. The first three amino acids and the last three amino acids of the CDR3 sequences from the TCRβ gene sequence can be removed. The cancer can be selected from the group consisting of leukemias, lymphomas, and hematologic malignancies.

An embodiment provides a method of predicting if an immune cell passes or fails immune cell selection for an immune cell receptor chain (TCR) comprising obtaining a test immune cell receptor chain gene including multiple gene segments; translating the test immune cell receptor chain gene into an immune cell receptor protein sequence, for each multiple gene segment, determining a gene feature that numerically represents one gene segment; for each amino acid included in the immune receptor protein sequence, determining a feature vector that numerically represents one amino acid; and determining, by a machine learning system, a selection prediction for an immune cell receptor chain based on the gene features for each of the multiple gene segments, the feature vectors for each of the amino acids in the immune cell receptor chain protein sequence, and a number of trained weights included in one or more models of the machine learning system.

The immune receptor chain gene can be selected from the group consisting of T cell receptor (TCR), TCR alpha chain (TCRα), TCR beta chain (TCRβ), TCR delta chain (TCRβ), TCR gamma chain (TCRγ), B cell receptor (BCR), BCR light chain (BCRL), BCR heavy chain (BCRH), immunoglobulin light chain (IgL), immunoglobulin heavy chain (IgH), immunoglobulin kappa chain (Igκ) and immunoglobulin lambda chain (Igλ). For example, the immune receptor chain gene can be a TCRβ gene. The gene segments can be selected from the group consisting of variable (V) gene segments, diversity (D) gene segments, joining (J) gene segments, and any combination thereof. The selection prediction can distinguish a TCRβ protein sequence of a productive TCRβ gene from a TCRβ protein sequence of a repaired TCRβ gene. The machine learning system can include an ensemble of multiple models, each model included in the ensemble of multiple models can generate an output and the outputs from each model can be combined to determine the selection prediction. The models included in the ensemble of multiple models can be arranged in a neural decision tree architecture that includes a hierarchical arrangement of more than two consecutive decisions. The hierarchical arrangement of more than two consecutive decisions can include a base decision at a first position in the hierarchical arrangement and a terminal decision at a last position in the hierarchical arrangement; and the neural decision tree architecture can include decisions composed of a committee of decisions aggregated together into a single decision using an arithmetic mean, wherein the number of decisions in each committee increases from the terminal decision in the neural decision tree to the base decision on the neural decision tree, herein also referred to as a neural committee tree (NCT). The method can further comprise obtaining a training dataset including a library of TCRβ genes and the TCRβ protein sequences of the TCRβ genes; and training the one or more models included in the machine learning system using the training dataset by fitting the trained weights included in each model using an optimization process. The library of TCRβ genes can include multiple productive genes and multiple non-productive genes. A non-productive TCRβ gene can be a TCRβ gene with out-of-frame gene segments or a TCRβ gene with a stop codon in a somatic junction between gene segments. A TCRβ gene encoding an amino acid sequence capable of antigen recognition can be identified as a productive TCRβ gene, and a TCRβ gene without an amino acid sequence capable of antigen recognition can be identified as a non-productive TCRβ gene. The method can further comprise repairing each of the multiple non-productive genes; and translating each of the repaired non-productive genes into a TCRβ protein sequence. Repairing a non-productive TCRβ gene can comprise adding or removing one or more nucleotides at a somatic junction between gene segments to bring the gene segments into a same reading frame and/or mutating a nucleotide in a somatic region between gene segments to convert a stop codon into an amino acid. Repairing a TCRβ gene identified as non-productive can comprise generating a repaired TCRβ gene. The library of TCRβ genes and TCRβ protein sequences can be obtained from a sample provided by an HLA-matched healthy donor. The sample can be peripheral blood or a tissue sample. The feature vector can include a piece of data related to a property of an amino acid, the property can be at least one of a polarity, one or more secondary structure associations, a molecular volume, a codon diversity, or an electrostatic charge. T cells isolated from a particular T cell subset can used. T cells can be isolated by cell sorting. T cells can be isolated by RNA expression. The subject can be human. Each of the repaired non-productive genes can be weighted according to the probability of that a repair used to generate a particular repaired non-productive gene appears naturally among the subject's non-productive genes. A TCRβ gene can be from non-regulatory T cells.

Another embodiment provides a method of predicting a risk of developing an autoimmune disease or disorder in a subject comprising a) reconstituting T cell selection in a matching healthy donor by classifying each T cell receptors (TCRβ) gene as a productive TCRβ gene or a repaired TCRβ using the machine learning system described herein, b) applying the T cell selection reconstituted from the healthy donor to T cells from the subject, and c), evaluating a number of escaped T cells in the subject that fail T cell selection in the healthy donor, wherein a number of escaped T cells higher than a threshold indicates a risk of having or of developing an autoimmune disease or disorder.

Reconstituting T cell selection in the healthy donor can comprise sequencing TCRβ genes in a sample from the matching healthy donor and classifying each T cell receptor (TCRβ) gene as a productive TCRβ gene or a repaired TCRβ using the machine learning system described herein. Applying the T cell selection reconstituted from the healthy donor to T cells from the subject can comprise sequencing TCRβ genes in a sample from the subject and classifying each TCRβ gene of the subject as a productive TCRβ gene or a repaired TCRβ gene. A healthy donor can be an HLA-matched healthy donor. The HLA-matched healthy donor can be a genetic relative of the subject. The sample from the matching healthy donor can be a biospecimen from the subject collected prior to the development of any symptom of a disease. The biospecimen can be banked blood. The biospecimen can be collected prior to an immune checkpoint inhibitor therapy.

An additional embodiment provides a method of predicting a risk of developing an autoimmune disease or disorder in a subject comprising a) reconstituting T cell selection in multiple healthy donors by classifying each TCRβ gene as a productive TCRβ gene or a repaired TCRβ using the machine learning system described herein, b) applying the T cell selection reconstituted from the healthy donors to T cells from the subject, and c) evaluating a number of escaped T cells in the subject that fail T cell selection in the healthy donor, wherein a number of escaped T cells higher than a threshold indicates a risk of having or of developing an autoimmune disease or disorder.

Reconstituting T cell selection in multiple healthy donors can comprise a) sequencing TCRβ genes in a sample from each donor, b) determining HLA type of each donor or sequencing MHC genes for each donor, c) tagging each TCRβ gene by the donor's HLA type, and d) classifying each TCRβ gene as a productive TCRβ gene or a repaired TCRβ gene, using the HLA tag as an additional feature for each TCRβ gene. Applying the T cell selection reconstituted from the healthy donors to the subject can comprise a) sequencing TCRβ genes in a sample from the subject, b) determining HLA type of the subject or sequencing MHC genes of the subject, c) tagging each TCRβ gene by the subject's HLA type, and d) classifying each TCRβ gene of the subject as a productive TCRβ gene or a repaired TCRβ gene. Escaped T cells can be T cells with a productive TCRβ gene misclassified as a repaired TCRβ gene. The sample can be peripheral blood or a tissue sample.

An embodiment provides a method of predicting a risk of developing alloimmunity from organ transplant in an organ recipient comprising a) reconstituting T cell selection in an organ donor by classifying each TCRβ gene as a productive TCRβ gene or a repaired TCRβ gene using the machine learning system described herein, b) applying the T cell selection reconstituted from the donor to the organ recipient, and c) determining a number of T cells from the organ recipient that are non-tolerant to an organ donor tissue, wherein a number of non-tolerant T cells in the organ recipient higher than a threshold indicates a risk of having or of developing an alloimmunity from organ transplant.

Reconstituting T cell selection in the organ donor can comprise sequencing TCRβ genes in a sample from the organ donor and classifying each TCRβ gene as a productive TCRβ gene or a repaired TCRβ gene using the machine learning system described herein. Applying the T cell selection reconstituted from the organ donor to the organ recipient can comprise sequencing TCRβ genes in a sample from the organ recipient and classifying each TCRβ gene as a productive TCRβ gene or a repaired TCRβ gene. Non-tolerant T cells can be T cells with a productive TCRβ gene misclassified as a repaired TCRβ gene. A non-tolerant T cell can be a T cell from the organ recipient that is predicted to fail T cell selection in the organ donor. The non-tolerant T cell can be a T cell from the organ recipient that is likely to drive an organ transplant rejection.

Another embodiment provides a method of predicting a risk of developing graft-versus-host disease (GvHD) from transplant or cells in a recipient comprising a) reconstituting T cell selection in a recipient by each TCRβ gene as a productive TCRβ gene or a repaired TCRβ gene using the machine learning system described herein; b) applying T cell selection reconstituted from the recipient to the donor, and c) determining a number of T cells from the donor that are non-tolerant to a recipient, wherein a number of non-tolerant T cells in the donor higher than a threshold indicates a risk of having or of developing GvHD from organ or cellular transplant.

Reconstituting T cell selection in the recipient can comprise sequencing TCRβ genes in a sample from the recipient and classifying each TCRβ gene as a productive TCRβ gene or a repaired TCRβ gene by using the machine learning system described herein. Applying T cell selection reconstituted from the recipient to the donor can comprise sequencing TCRβ genes in a sample from the donor and classifying each TCRβ gene as a productive TCRβ gene or a repaired TCRβ gene. Non-tolerant T cells can be T cells with a productive TCRβ gene misclassified as a repaired TCR gene. A non-tolerant T cell can be a T cell from the donor that is predicted to fail T cell selection in the recipient. The non-tolerant T cell can be a T cell from the donor that is likely to drive GvHD. The sample from the donor can be a sample from the transplant. The sample from the recipient can be peripheral blood or a tissue sample.

An additional embodiment provides a method of predicting a risk of developing alloimmunity from an adoptive T cell therapy in a recipient comprising a) reconstituting T cell selection in a recipient by classifying each TCRβ gene as a productive TCRβ gene or a repaired TCRβ gene using the machine learning system described herein, b) applying T cell selection reconstituted from the recipient to the donor T cells, and c) determining a number of T cells from the donor being donated that are non-tolerant to the recipient, wherein a number of non-tolerant T cells in the donor higher than a threshold indicates a risk of having or of developing alloimmunity from an adoptive T cell therapy.

Reconstituting T cell selection in the recipient can comprise sequencing TCRβ genes in a sample from the donor and classifying each TCRβ gene as a productive TCRβ gene or a repaired TCRβ gene using the machine learning system described herein. Applying T cell selection reconstituted from the recipient to the donor T cells can comprise sequencing TCRβ genes in a sample of the donated T cells from the donor and classifying each TCRβ gene as a productive TCRβ gene or a repaired TCRβ gene. Non-tolerant T cells can be T cells with a productive TCRβ gene misclassified as a repaired TCRβ gene. A non-tolerant T cell can be a T cell from the donor that is predicted to fail T cell selection in the recipient. The non-tolerant T cell can be a T cell from the donor that is likely to drive alloimmunity in the recipient. Alloimmunity from an adoptive T cell therapy can comprise unwanted immune attacks from the donor T cells against the recipient's cells and tissues. The sample can be peripheral blood or a tissue sample. Adoptive T cells in the adoptive T cell therapy can be allogenic CAR T cells. Adoptive T cells in the adoptive T cell therapy can be allogenic T cells with an engineered TCR.

Another embodiment provides a method of predicting compatibility of an engineered T cell receptor (TCRβ) therapy in a recipient comprising: a) reconstituting T cell selection in a recipient by classifying each TCRβ gene as a productive TCRβ gene or a repaired TCRβ gene using the machine learning system described herein, b) applying the T cell selection reconstituted from the recipient to the engineered TCRβ, and c) determining if the engineered TCRβ is non-tolerant to the recipient, thereby predicting compatibility to an engineered TCRβ therapy.

Reconstituting T cell selection in the recipient can comprise sequencing T cell receptors (TCRβ) genes in a sample from the recipient and classifying each TCRβ gene as a productive TCRβ gene or a repaired TCRβ gene. Applying the T cell selection from the recipient to the engineered TCR can comprise classifying the engineered TCRβ gene as a productive TCRβ gene or a repaired TCRβ gene. A Non-tolerant engineered TCRβ gene can be a productive TCRβ gene misclassified as a repaired TCRβ gene. A non-tolerant engineered TCRβ is predicted to fail T cell selection in the recipient. The non-tolerant engineered TCRβ is likely to drive alloimmunity in the recipient. Alloimmunity from an engineered TCRβ therapy can comprise unwanted immune attacks from the T cells with an engineered TCRβ against the recipient's cells and tissues. The sample can be peripheral blood or a tissue sample.

An embodiment provides a method of predicting a risk of developing an autoimmune disease or disorder in a subject comprising a) reconstituting B cell selection in the healthy subjects by classifying each B cell receptor (BCR) genes as a productive BCR gene or a repaired BCR gene using the machine learning system described herein, wherein the immune receptor chain gene is a BCR gene, b) applying the B cell selection reconstituted from the healthy donors to B cells from the subject, and c) evaluating a number of escaped B cells in the subject that fail B cell selection in the healthy donor, wherein a number of escaped B cells higher than a threshold indicates a risk of having or of developing an autoimmune disease or disorder.

The gene segments can be selected from the group consisting of variable (V) gene segments, diversity (D) gene segments, joining (J) gene segments, and any combination thereof. The selection prediction can identify a BCR gene as a productive BCR gene or a repaired BCR gene. The machine learning system can include an ensemble of multiple prediction models, each prediction model included in the ensemble of multiple prediction models can generate a model prediction and the model predictions from each prediction model can be combined to determine the selection prediction. A modified neural decision tree architecture including a hierarchical arrangement of more than two consecutive decisions can be used to aggregate the model predictions into the selection prediction. The neural decision tree architecture can include decisions composed of a committee of decisions aggregated together into a single decision using an arithmetic mean, wherein the number of decisions in each committee increases from the terminal decision in the neural decision tree to the base decision on the neural decision tree, herein also referred to as a neural committee tree (NCT). The method can further comprise obtaining a training dataset including a library of BCR genes and the BCR protein sequences of the BCR genes; and training the one or more prediction models included in the machine learning system using the training dataset by determining the weight values included in each prediction model using an optimization process. The library of BCR genes can include multiple productive genes and multiple non-productive genes. A non-productive BCR gene can be a BCR gene with out-of-frame gene segments or a BCR gene with a stop codon in a somatic junction between gene segments. The method can further comprise repairing each of the multiple non-productive genes; and translating each of the repaired non-productive genes into a BCR protein sequence. Repairing non-productive BCR gene can comprise adding or removing one or more nucleotides at a somatic junction between gene segments to bring the gene segments in a same reading frame and/or mutating a nucleotide in a somatic region between gene segments to convert a stop codon into an amino acid. Repairing a BCR gene identified as non-productive can comprise generating a repaired BCR gene. The library of BCR genes and BCR protein sequences can be obtained from a sample provided by an HLA-matched healthy donor. The protein feature can include a piece of data related to a property of an amino acid, the property can be at least one of a polarity, one or more secondary structure associations, a molecular volume, a codon diversity, or an electrostatic charge. Each of the repaired non-productive genes can be weighted according to a probability that a repair used to generate a particular repaired non-productive gene appears naturally among the subject's non-productive genes. Reconstituting B cell selection in healthy subjects can comprise sequencing B cell receptor (BCR) genes in a sample from the healthy subjects and classifying each BCR gene of the healthy subjects as a productive BCR gene or a repaired BCR gene. Applying the B cell selection reconstituted from the healthy donors to B cells from the subject can comprise sequencing BCR genes in a sample from the subject and classifying each BCR gene as a productive BCR gene or a repaired BCR gene. Escaped B cells can be B cells with a productive BCR gene misclassified as a repaired BCR gene. The sample can be peripheral blood or a tissue sample.

An embodiment provides a method of predicting an antibody drug safety in a subject comprising a) reconstituting B cell selection in the subject by classifying each BCR gene of the subject as a productive BCR gene or a repaired BCR gene using the machine learning system described herein, wherein the immune receptor chain gene is BCR gene, and b) determining if a BCR gene encoding the antibody drug is tolerant to subject's self-antigens, wherein a tolerant BCR gene encoding an antibody drug is a BCR gene correctly classified as a productive BCR gene.

The gene segments can be selected from the group consisting of variable (V) gene segments, diversity (D) gene segments, joining (J) gene segments, and any combination thereof. The selection prediction can identify BCR gene as a productive BCR gene or a repaired BCR gene. Reconstituting B cell selection in the subject can comprise sequencing BCR genes in a sample from the subject and classifying each BCR gene of the subject as a productive BCR gene or a repaired BCR gene using the machine learning system described herein. A non-tolerant BCR gene encoding an antibody drug can be a BCR gene misclassified as a repaired BCR gene. A non-tolerant BCR gene encoding an antibody drug can be a BCR gene that is predicted to fail B cell selection in the subject. The non-tolerant BCR gene encoding an antibody drug can encode an antibody drug that is likely to bind self-antigens in the subject. An antibody drug classified as likely to bind self-antigen can indicate a lack of safety of use of the antibody drug in the subject. The sample can be peripheral blood or a tissue sample.

Another embodiment provides a method of predicting a risk of developing alloimmunity from a chimeric antigen receptor (CAR)-T cell therapy in a subject comprising determining if an antigen binding domain of the CAR is tolerant to subject's self-antigens, wherein determining if an antigen binding domain of the CAR is tolerant to subject's self-antigens comprises a) reconstituting B cell selection in the subject by classifying each BCR gene of the subject as a productive BCR gene or a repaired BCR gene using the machine learning system described herein, wherein the immune receptor chain gene is BCR gene, and b) determining if a BCR gene encoding the antigen binding domain of the CAR is tolerant to subject's self-antigens, wherein a tolerant BCR gene encoding the antigen binding domain of the CAR is a BCR gene correctly classified as a productive BCR gene.

Reconstituting B cell selection in a subject can comprise sequencing BCR genes in a sample from the subject and classifying each BCR gene of the subject as a productive BCR gene or a repaired BCR gene. A non-tolerant BCR gene encoding the antigen binding domain of the CAR can be a BCR gene misclassified as a repaired BCR gene. A non-tolerant BCR gene encoding the antigen binding domain of the CAR can be a BCR gene that is predicted to fail B cell selection in the subject. The non-tolerant BCR gene encoding an antibody drug can encode an antibody drug that is likely to bind self-antigens in the subject. A BCR gene classified as likely to bind self-antigen can indicate a lack of safety of use of the CAR-T cell therapy in the subject. The sample can be peripheral blood or a tissue sample.

Therefore, provided herein are unconventional methods of determining an organ donor/organ recipient compatibility, cellular donor/cellular recipient compatibility, and other predictive methods using, inter alia, an unconventional step of classifying immune receptor chain genes that relies on making a repair or repairs to the non-productive immune receptor chain genes. The unique methodology can be used in, for example, methods of determining an organ donor/organ recipient compatibility, cellular donor/cellular recipient compatibility, methods of predicting a risk of developing an autoimmune disease, methods of predicting a risk of developing alloimmunity from organ or cellular transplant in a recipient, methods of predicting a risk of developing graft-versus-host disease (GvHD) from organ or cellular transplant in a recipient, methods of predicting cancer relapse in a hematopoietic stem cell recipients, methods of predicting a risk of developing alloimmunity from an adoptive T cell therapy in a recipient, methods of predicting compatibility of an engineered T cell receptor (TCR) therapy in a recipient, methods of predicting an antibody drug safety in a subject, and methods of predicting a risk of developing alloimmunity from a chimeric antigen receptor (CAR)-T cell therapy in a subject using a machine learning system that relies on the reconstitution of T cell selection. The use of these methods provides for greater accuracy in determining organ/cellular donor and recipient compatibly and other predictions using unique technical steps including, among others, the generation of non-naturally occurring repaired immune receptor chain genes from non-productive or non-functional immune receptor chain genes. The generation of non-naturally occurring repaired immune receptor chain genes for use in these types of methods is not currently routine or known in the art.

### BRIEF DESCRIPTION OF THE DRAWINGS

The accompanying drawings are included to provide a further understanding of the methods and compositions of the disclosure, are incorporated in, and constitute a part of this specification. The drawings illustrate one or more embodiments of the disclosure, and together with the description serve to explain the concepts and operation of the disclosure.

FIGS. 1A-1C illustrate the TCR recombination process and the TCR selection processes. FIG. 1A illustrates the genome multiple V, D, and J gene segments. During V(D)J recombination, the genome is cut and ligated to pair individual V, D (β-chain only), and J gene segments. Deletions and insertions introduce random nucleotides at the junctions between gene segments. The TCR gene expresses on the surface of the T cell as a protein. FIG. 1B illustrates how T cells are culled by positive and negative selection based on the expressed TCR establishing immune tolerance. FIG. 1C illustrates how non-productive TCR genes do not express because the V and J segments are in different open reading frames (top) or because of a stop codon (middle). V(D)J recombination on the alternate chromosome can result in a second TCR gene, which may express a receptor, thereby allowing the T cell to survive T cell selection.

FIG. 2 illustrates an exemplary method of predicting T cell selection outcomes.

FIGS. 3A-3B illustrate exemplary alterations that are made to repair non-productive TCR genes.

FIG. 4 illustrates an exemplary machine learning system for predicting T cell selection outcomes.

FIGS. 5A-5B illustrate an exemplary neural committee tree architecture.

FIG. 6 illustrates more details of the predictions models included in the machine learning system of FIG. 4.

FIGS. 7A-7C illustrate results from an exemplary T cell selection simulation analysis performed using TCR genes from a single mouse subject.

FIG. 8 illustrates results from an exemplary T cell selection simulation performed using TCR genes from mature T cells.

FIGS. 9A-9C illustrate results from an exemplary B cell selection simulation preformed using BCR genes from naïve B cells.

FIG. 10 illustrates the comparison of T cells before and after T cell selection used to determine donor-recipient compatibility.

FIG. 11 illustrates how the sequenced TCRβ genes are used to mimic TCRβ before and after T cell selection.

FIG. 12A is a Venn diagram illustrating how PSF_{DONOR-PROD} is calculated.

FIG. 12B is a graph illustrating PSF_{DONOR-PROD} for aGvHD cases and controls. Each column is a different transplant.

FIG. 12C is a ROC curve illustrating that moving the cutoff from FIG. 12B changes the true and false positive rates.

FIG. 13A is a Venn diagram illustrating how PSF_{DONOR-REPAIR} is calculated.

FIG. 13B is a graph illustrating PSF_{DONOR-REPAIR} for cGvHD cases and controls. Each column is a different transplant.

FIG. 13C is a ROC curve illustrating that moving the cutoff from FIG. 13B changes the true and false positive rates.

FIG. 14 is a Venn diagram revealing the number of TCRβs shared between a donor and recipient. Translating nucleotide sequences to protein sequences reveals different TCRβ genes encoding identical TCRβs. The first and last three amino acid residues from each CDR3 protein sequence are trimmed because these residues do not contact antigen. TCRβs are considered equivalent if the trimmed CDR3 protein sequences are identical.

FIGS. 15A-15C illustrate that TCRβ gene sequences reveal germline encoded V, D, J gene segments as well as somatic alterations that occur during V(D)J recombination. FIG. 15A shows that productive TCRβ genes found in peripheral blood can be translated to an amino acid sequence. FIG. 15B shows that TCRβ genes found in peripheral blood with out-of-frame V and J gene segments do not express a functioning receptor for T cell selection. This example of a non-productive TCRβ gene can be repaired by deleting somatic nucleotides. FIG. 15C shows that TCRβ genes found in peripheral blood encoding a stop codon in a somatic junction also do not express a functioning receptor for T cell selection. This example of a non-productive TCRβ gene can be repaired by modifying somatic nucleotides.

FIG. 16A is a Venn diagrams illustrating how PSF_{AUTO} is calculated.

FIG. 16B is a graph illustrating PSF_{AUTO} for autologous skin (square, triangle), PBMC (circle), and thymus (diamond) samples. Each column is a different patient. PSF_{AUTO} values for thymus, which contains developing T cells before T cell selection, is lower than skin and PBMC, which contain mature T cells after T cell selection. The cutoff (dashed line) distinguishes TCRβ populations before and after T cell selection and is almost identical to the cutoff used to distinguish aGvHD cases and controls.

FIG. 17A is a Venn diagram illustrating donor TCRβs lacking from the recipient, denoted f_{NOVEL}.

FIG. 17B is a graph illustrating f_{NOVEL} for cancer relapse cases and controls. Each column represents a different recipient.

FIG. 17C is a ROC curve illustrating that moving the cutoff from FIG. 17B changes the true and false positive rates.

FIG. 18 is a graph illustrating predictions for aGvHD plotted against predictions for relapse. The cutoffs correctly identify 3/7≈43% of recipients that avoid both aGvHD and relapse. Without cutoffs, 6/17≈35% of recipients avoid both aGvHD and relapse.

FIG. 19 is a graph illustrating predictions for cGvHD plotted against predictions for cancer relapse. The cutoffs correctly identify 5/5≈100% of recipients that avoid both cGvHD and relapse. Without cutoffs, 8/17≈47% of recipients avoid both cGvHD and relapse.

FIG. 20 is a schematic illustrating how predictions for aGvHD, cGvHD, and cancer relapse can be used to screen candidates for the best donor.

### DETAILED DESCRIPTION

The present disclosure provides method of predicting if a T cell passes or fails T cell selection for a T cell receptor (TCR) implemented in a machine learning system, and methods of use thereof. The methods of use include methods of predicting a risk of developing an autoimmune disease or disorder in a subject, methods of predicting a risk of developing alloimmunity from organ transplant in an organ recipient, methods of predicting a risk of developing graft-versus-host disease (GvHD) from organ or cellular transplant in a recipient, methods of predicting a risk of developing alloimmunity from an adoptive T cell therapy in a recipient, methods of predicting an antibody drug safety in a subject, and methods of predicting a risk of developing alloimmunity from a chimeric antigen receptor (CAR)-T cell therapy in a subject.

Overview

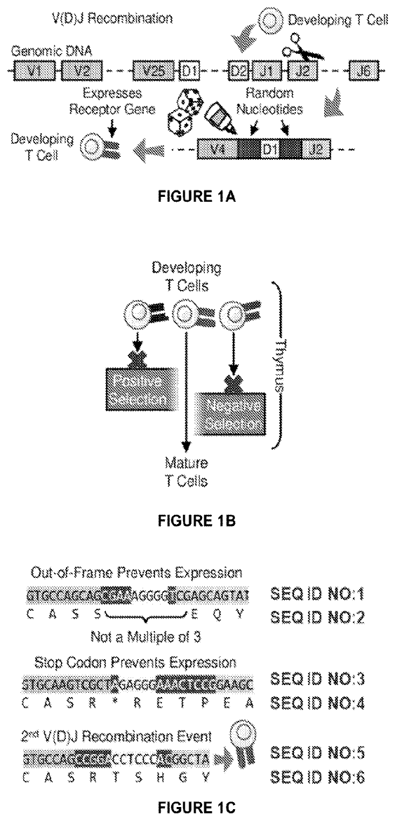

By a process known as V(D)J recombination, developing T cells edit their DNA to assemble de-novo TCR genes. From dozens of variable (V), diversity (D), and joining (J) gene segments, a TCR gene is formed by directly editing the genome to couple individual V, D, and J segments into a complete gene (FIG. 1A). When segments are ligated together, consecutive deletions and insertions introduce random nucleotides at the junctions between segments, creating additional alterations in the TCR gene. Thus, each TCR gene contains somatically rearranged germline segments with consecutive somatic alterations forming the junctions between these segments. By generating a potentially unique TCR gene, each T cell can potentially express a distinct TCR. When confronted with a new antigen, a large population of T cells will, by chance, contain a TCR that can bind that antigen.

TCR genes are created without regard for which antigens the TCR can bind, making it essential that developing T cells undergo T cell selection. The two major stages of T cell selection are positive and negative selections, which take place in that order in the thymus (FIG. 1B). During positive selection, developing T cells that bind MHC receive a survival signal, ensuring that the surviving T cells are capable of functionally interacting with antigen presented by MHC. Positive selection establishes MHC as designated zones where T cells surveil for antigen. During negative selection, developing T cells expressing TCRs that strongly bind self-antigens receive an apoptotic signal leading to cell death, ensuring that the surviving T cells do not recognize self-antigens. Negative selection culls developing T cells that would drive an autoimmune attack against healthy cells and tissue. Each T cell receptor (TCR) gene is created without regard for which substances (antigens) the receptor can recognize. T cell selection culls developing T cells when their TCRs (i) fail to recognize major histocompatibility complexes (MHCs) that act as antigen presenting platforms or (ii) recognize with high affinity self-antigens derived from healthy cells and tissue. Both positive and negative selection are probabilistic processes without guaranteed outcomes and developing T cells with identical TCRs can have opposite outcomes during T cell selection. T cells that complete the selection process migrate out of the thymus to other organ sites, such as the spleen, as mature T cells.

Early attempts to sequence TCR genes revealed mature T cells with non-productive TCR genes unable to express a functioning TCR because the (i) V and J segments were in different open reading frames or (ii) a stop codon was found in the junctions between gene segments (FIG. 1C). Without a functioning TCR, these T cells would be culled by T cell selection rather than reach maturity, so the appearance of non-productive TCR genes in mature T cells would seem like a conundrum. However, V(D)J recombination taking place on the alternate chromosome can result in a partner TCR gene expressing a functioning TCR, allowing the T cell to survive T cell selection. Because the fate of the T cell during the selection process depends solely on the partner TCR gene, the non-productive TCR gene remains independent of T cell selection, representing the types of TCRs that would appear in the absence of T cell selection. Therefore, comparisons of productive to non-productive TCR genes can reveal information about the TCR genes culled by T cell selection. Previous studies have found that T cell selection restricts TCR genes by sequence length and V(D)J rearrangements.

Described herein are methods based on high throughput TCR sequencing and machine learning system that uses the TCR gene to predict which T cells are culled. Using this system, T cell selection can be reconstituted in-silico for any individual. The in-silico methods can be used to uncover patterns in TCR protein sequences that influence whether a T cell is culled.

Allogenic hematopoietic stem cell transplantation (allo-HSCT) is an important treatment option for various types of leukemias, lymphomas, and other hematologic malignancies. However, its use is associated with significant morbidity and mortality with 9-15% of allo-HSCT recipients dying from graft-vs-host disease (GvHD) and another 23% from cancer relapse. Reducing allo-HSCT morbidity and mortality is important because (i) new cancer immunotherapies are reducing and delaying but not eliminating the need for allo-HSCT, and (ii) wider use of cyclophosphamide has reduced but does not eliminate GvHD. Despite the challenges of allo-HSCT and the emergence of alternative treatments, the annual number of allo-HSCTs has consistently increased over the past two decades, suggesting allo-HSCT will remain an indispensable treatment for hematologic malignancies for the foreseeable future.

Although not the goal of an allo-HSCT, T cells residing with hematopoietic stem cells (HSC) are also transplanted into the recipient and develop later from donor HSC in the recipient. T cells are an important part of the transplant because donor T cells sometimes recognize the recipient's cancer, thereby protecting against cancer relapse. However, it is crucial to match the donor and recipient because incompatible donor T cells will cause immune attacks against the recipient, thereby leading to graft-vs-host disease (GvHD).

Current approaches for identifying donor-recipient matches for allo-HSCT only partially determine T cell compatibility. For example, HLA typing determines if the donor and recipient share the same major histocompatibility complexes (MHCs) during the first stage of T cell selection, but this leaves the second stage of T cell selection untyped, potentially explaining why 40% of identically matched related donors still develop GvHD. Minor histocompatibility antigen (mHA) typing attempts to close this gap by determining if the donor and recipient express the same self-antigens, but mHA typing can only match a few hundred of the millions of self-antigens that can cause GvHD, potentially explaining why mHA typing fails to predict GvHD. Finally, mixed lymphocyte reactions (MLRs) determine if donor T cells adversely interact with recipient lymphocytes, but adverse reactions can take place in other tissues not tested, potentially explaining the reasons behind the failure of MLRs to predict GvHD.

To determine donor-recipient compatibility, donor and recipient T cells can be compared before and after T cell selection (also known as thymic selection) because this is the immunological process that determines T cell compatibility. As illustrated in FIG. 10, hematopoietic stem cells (HSC) in the bone marrow produce developing T cells that undergo T cell selection in the thymus. T cell selection removes developing T cells that are not MHC restricted or that strongly recognize self-antigens. The T cells that survive migrate to peripheral blood as mature T cells compatible with the host.

A compatible donor would delete the same types of T cells as the recipient during T cell selection, ensuring the donor T cells are already compatible with the recipient. During T cell selection, incompatible T cells are removed based on their expressed TCR. Therefore, the TCRs can be used to check for compatibility. Described herein, is a demonstration that the quantification of compatible donor T cells, as predicted by their TCRs, can be utilized as a marker for predicting GvHD. This information can be used to select a donor or a specific GvHD prophylactic strategy.

Immune Cell Receptor Classification and Repairing of Non-Productive Receptors

FIG. 1A illustrates the VDJ recombination process that leads to the expression at the surface of immune cells of a variety of possible immune receptors (due to the recombination and the addition/deletion of random nucleotides). Among the multitude of possible immune receptors generated, some can comprise out-of-frame events in their protein sequence which prevent the expression of the receptor (e.g., the number of nucleotides is not a multiple of 3), some can present a premature stop codon, which also prevent the expression of the receptor. In the absence of such events, a receptor can be expressed at the surface of the immune cell (see FIG. 1C). FIG. 10 illustrates the immune cell selection process, using T cell selection as an example. In the bone marrow, developing T cells are present and no selection has occurred. The obtaining of mature T cells (T cells remaining after T cell selection), T cells that are not MHC restricted are removed (such as those cells that do not express an immune receptor at their surface), and self-antigen reactive T cells are removed.

The present disclosure relies on the discovery that the gene sequence of an immune receptor can be obtained from a sample, the gene sequence can be translated into a protein sequence or an attempt made thereof, and the analysis of the protein sequence can be used to identify immune receptor chain genes encoding an amino acid sequence capable of antigen recognition which corresponds to productive immune receptor genes or immune receptor chain genes and to identify immune receptor genes or immune receptor chain genes without an amino acid sequence not capable of antigen recognition which correspond to non-productive immune receptor genes or immune receptor chain genes (see FIGS. 10 and 11). The method then relies on repairing the sequences of immune receptor chain genes identified as non-productive to generate a repaired immune receptor chain gene.

A functional immune receptor, such as a functional TCR is a TCR that has an amino acid rendering the TCR capable of recognizing an antigen. Antigen recognition, as used herein refers to the capability of an immune receptor to functionally interact with an antigen when it is presented by an antigen presenting complex such as an MHC for example. A productive TCR, as used herein can refer, without different in the meaning to either a functional TCR (i.e., that has an amino acid sequence rendering the TCR capable of antigen recognition), or to a TCR that has an amino acid sequence that does not present an out-of-frame VDJ recombination, nor a stop codon.

The immune receptor chain gene sequence can comprise multiple gene segments e.g., variable (V) gene segments, diversity (D) gene segments, joining (J) gene segments, and any combination thereof.

Not all immune receptor genes contain a D gene segment. For example, TCR alpha, TCR delta, BCRL, IgL, and Igκ do not contain D genes. Also, in some cases, somatic alterations can completely remove the D gene from TCR beta, TCR gamma, BCRH, and IgH genes. Accordingly, the immune receptor chain describes herein can comprise multiples gene segments including V, D and J gene segments, or a combination thereof depending on the recombination and somatic alterations.

An immune receptor is encoded by two immune receptor gene chains. The method described herein generally refer to one immune receptor gene chain at a time and can be applied for any immune receptor gene chain. Without wanting to limit any of the methods presented herein, it is to be understood that to be reflective of a complete immune receptor, the methods described herein can be applied to each chain of an immune receptor, using the methods described herein for each single chain. As used herein, repairing the immune receptor chain genes can include repairing the full immune receptor. The immune receptor chain gene can be any immune cell receptor, including but not limited to those selected from the group consisting of T cell receptor (TCR), TCR alpha chain (TCRα), TCR beta chain (TCRβ), TCR delta chain (TCRβ), TCR gamma chain (TCRγ), B cell receptor (BCR), BCR light chain (BCRL), BCR heavy chain (BCRH), immunoglobulin light chain (IgL), immunoglobulin heavy chain (IgH), immunoglobulin kappa chain (Igκ) and immunoglobulin lambda chain (Igλ). For example, the immune receptor chain gene can be TCRβ.

The methods described herein provide for repairing non-productive immune receptor genes. That is the methods provide for the identification of immune receptor genes that are not selected during the immune cell selection process, and therefore that are not expressed at the surface of immune cells in a subject. Repairing non-productive immune receptor genes has multiples applications as described herein, e.g., it can be used to compare the immune cell receptor selection process in matched subjects, and to predict for example, adverse events associated with immune cells (e.g., organ rejection, graft versus host disease, cancer relapse, etc.). Repairing non-productive immune cell receptor chain genes, e.g., TCRβ genes, can comprise modifying the nucleotide sequence of said TCRβ genes to obtain a sequence that would otherwise be classified as productive. Non-productive TCRβ genes can be TCRβ genes with out-of-frame gene segments or TCRβ genes with a stop codon in a somatic junction between gene segments and somatic alterations. Therefore, repairing non-productive TCRβ genes can comprise adding or removing one or more nucleotides at a somatic junction between gene segments to bring the gene segments into a same reading frame or mutating a nucleotide in a somatic region between gene segments to convert a stop codon into an amino acid.

Non-productive TCRβ genes can include TCRβ genes that do not express a TCRβ capable of antigen recognition. The repairing of TCRβ genes described herein (e.g., modifying the sequence of an immune receptor) can result in the generation of an immune receptor that has an amino acid sequence capable of antigen recognition. Repairing non-productive TCRβ genes can comprise bringing the V and J segments into the same reading frame, without bringing the reading frame of the D segment into the same reading frame. As used herein, “bring genes fragments into a same reading frame” can include adding or removing one or more nucleotides at a somatic junction between genes segments to bring the gene segments in a same reading frame. One or more nucleotides can include one or two nucleotides, that can be added or removed such that the reading frame is restored.

It is to be understood that the methods described herein generally rely on the use of the minimal number of sequence modifications to repair the immune receptor chain genes. That is, the method generally relies on the addition or the deletion of one or two nucleotides to bring gene fragments into a same reading frame, or to the mutation of one amino acid to remove a stop codon from an amino acid sequence. However, in some instances, the initial modification can induce a secondary event (or a third event, or a fourth event) that might require a second (or a third, or a fourth) modification to obtain an amino acid sequence that encodes a receptor chain capable of antigen recognition. For example, an addition or a deletion of one or two nucleotides to bring two gene fragments in a same reading frame can lead to the generation of a stop codon in the amino acid sequence and prevent the generation of an immune receptor capable of amino acid recognition. In a second repair, the stop codon would be removed. While it is possible to repair the immune receptor genes using more than one repair, it is to be understood that the more modifications are introduced into the sequences, the more artificial and foreign from the initial sequence the receptor becomes. This can be associated with a deterioration of the quality of the predictions that can be made using the methods described herein.

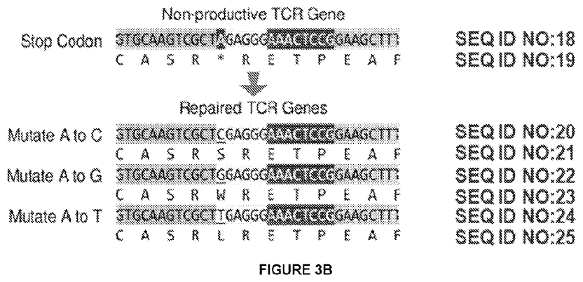

There are multiple ways to repair a non-productive immune receptor gene (see FIG. 3) because there are usually multiple somatic alterations. All the different ways to repair the gene are valid. Each repair can only require a single correction (i.e., removing 1 nucleotide, removing 2 nucleotides, adding a nucleotide, adding a first and second nucleotide, or mutating a nucleotide). In an embodiment one repair comprises removing 1 nucleotide, removing 2 nucleotides, adding a nucleotide, or mutating a nucleotide to bring gene segments into a same reading frame or to change a stop codon to a codon for an amino acid. In the methods described herein, a receptor that would require more than one modification to be repaired is not considered in the analysis of the immune receptor. In an embodiment, a receptor that would require more than two, or more than three, or more than four modifications to be repaired is not considered in the analysis of the immune receptor. In the methods described herein, the repair of the immune receptor can include the repair of the immune receptor chain gene sequence (e.g., nucleic acid sequence or amino acid sequence) after the VDJ recombination events; therefore the repair of the immune receptor chain gene sequences is not directed at the repair or germline genes.

A TCRβ gene sequence can comprise a complimentary determining region 1 (CDR1) sequence of the TCRβ gene, a CDR2 sequence of the TCRβ gene, a CDR3 sequence of the TCRβ gene, a combination thereof, or a sequence of a complete TCRβ gene. For example, the TCRβ gene sequence can be a CDR3 sequence of the TCRβ gene.

A TCRβ gene sequence use for the classification method described herein can be the entire TCRβ gene sequence, or any fragment thereof. For example, TCRβ gene sequence can comprise the entire TCRβ gene sequence minus the first three amino acids and the last three amino acids of the CDR3 sequences that can be removed from the TCRβ gene sequence.

Obtaining a TCRβ gene sequence can comprise sequencing TCRβ genes is any sample from a subject. For example, the sample can be a biological sample containing immune cells, for example T cells. The sample can be a blood sample from a subject. The blood sample can be a peripheral blood mononucleated cell sample.

Immune cells can be isolated from the sample prior to sequencing the immune cell receptor genes. For example, T cells can be isolated from a sample. Isolating T cells can be by cell sorting and/or RNA expression.

T cells can be any T cells, including but not limited to conventional adaptive T cells (including helper CD4+ T cells, cytotoxic CD8+ T cells, memory T cells, and regulatory CD4+ T cells) or innate-like T cells (including natural killer T cell and mucosal associated invariant T cells). For example, T cells can be non-regulatory T cells.

The subject can be a mammal such as a human.

Methods of Uses

The classification of immune cell receptors described herein can be used in a variety of applications, including, but not limited to determining an organ donor/organ recipient compatibility, predicting graft versus host disease (GvHD) in a recipient, and predicting cancer relapse in a subject (see FIG. 20)

Methods of Determining an Organ Donor/Organ Recipient Compatibility are Provided.

The method can comprise classifying TCRβ genes of the organ donor and TCRβ genes of the organ recipient as productive TCRβ genes or repaired TCRβ genes using the method described herein; comparing a number of productive and repaired TCRβ genes in a donor to a number of productive TCRβ genes in a recipient; and quantifying the fraction of TCRβ genes from the organ recipient that are compatible with the organ donor, thereby determining an organ donor/organ recipient compatibility.

Comparing can comprise calculating a post selection fraction score, denoted PSF_{RECIPIENT}, wherein the PSF_{RECIPIENT} score is a ratio between F_{PROD} and F_{TOTAL}, wherein F_{TOTAL} is F_{REPAIR}+F_{PROD}, and wherein F_{PROD} is a number of TCRβ genes identified as productive TCRβ genes in both the organ recipient and the organ donor, and F_{REPAIR} is a number of TCRβ genes identified as repaired TCRβ genes in the organ donor and identified as productive TCRβ genes in the organ recipient. The PSF_{RECIPIENT} can range from 0 to 1. A PSF_{RECIPIENT} of zero can indicate that none the TCRβ genes sequenced in the organ recipient are compatible with the organ donor. A PSF_{RECIPIENT} score of 1 can indicate that all the TCRβ genes sequenced in the organ donor are compatible with the organ recipient. The TCRβ gene sequence can comprise a CDR3 sequence of the TCRβ gene. The first three amino acids and the last three amino acids of the CDR3 sequences from the TCRβ gene sequence can be removed.

A PSF_{RECIPIENT} score equal to or greater than 0.81 can be indicative of a compatibility between the organ donor and the organ recipient. Alternatively, a PSF_{RECIPIENT} score lesser than 0.81 can be indicative of an incompatibility between the organ donor and the organ recipient.

A favorable score can be defined as a score that would be interpreted, by a physician or another health care professional responsible for assessing the compatibility of an organ recipient and an organ donor, as in favor of a transplant of the organ from the donor to the recipient. An unfavorable score can be defined as a score that would be interpreted as not in favor of the transplant of the organ from the donor to the recipient. The method described herein can further include the treatment of the organ recipient, which generally comprises the transplant of an organ from the organ donor to the organ recipient. As described herein, the treatment is to be administered to the organ recipient, when the score determined by the method described herein is favorable.

Methods of Predicting Graft Versus Host Disease (GvHD) in a Recipient

Methods of predicting graft versus host disease (GvHD) in a recipient are provided.

The methods can comprise classifying T cell receptor β (TCRβ) genes of the donor and TCRβ genes of the recipient as productive TCRβ genes or repaired TCRβ genes using the method described herein; comparing a number of productive and repaired TCRβ genes in the recipient to a number of productive TCRβ genes in the donor; and quantifying the fraction of TCRβ from the donor that are compatible with the recipient, thereby predicting GvHD in a recipient.

The GvHD can be acute GvHD (aGvHD) or chronic GvHD (cGvHD).

The organ or cells can bone marrow or a hematopoietic stem cell transplant.

The TCRβ gene sequence can comprise a CDR3 sequence of the TCRβ gene. The first three amino acids and the last three amino acids of the CDR3 sequences from the TCRβ gene sequence can be removed.

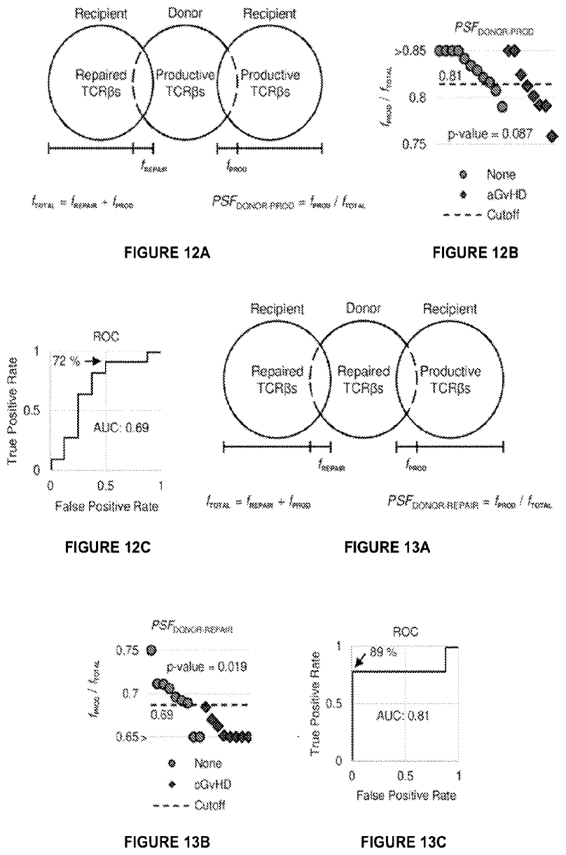

Predicting aGvHD can comprise quantifying a number of productive TCRβ gene from the donor that are compatible with the recipient. Quantifying a number of productive TCRβ genes from the donor that are compatible with the recipient can comprise calculating a post selection fraction score, denoted PSF_{DONOR-PROD}, wherein the PSF_{DONOR-PROD} score is a ratio between F_{PROD} and F_{TOTAL}, wherein F_{TOTAL} is F_{REPAIR}+F_{PROD}, and wherein F_{PROD} is a number of TCRβ genes identified as productive TCRβ genes in both the donor and the recipient, and F_{REPAIR} is a number of TCRβ genes identified as repaired TCRβ genes in the recipient and identified as productive TCRβ genes in the donor. (See FIG. 12A)

A PSF_{DONOR-PROD} score equal to or greater than 0.81 can be indicative of a compatibility between the donor and the recipient. Alternatively, a PSF_{DONOR-PROD} score less than 0.81 can be indicative of an incompatibility between the donor and the recipient, and a likelihood of the recipient to develop aGvHD. For example, a PSF_{DONOR-PROD} score less than the range of about 0.8 to 0.83 can be used to predict aGvHD.

Predicting cGvHD can comprise quantifying a number of repaired TCRβ genes from the donor that are compatible with the recipient. Quantifying a number of repaired TCRβ genes from the donor that are compatible with the recipient can comprise calculating a post selection fraction score, denoted PSF_{DONOR-REPAIR}, wherein the PSF_{DONOR-REPAIR} score is a ratio between F_{PROD} and F_{TOTAL}, wherein F_{TOTAL} is F_{REPAIR}+F_{PROD}, and wherein F_{PROD} is a number of TCRβ genes identified as productive TCRβ genes in the recipient and identified as repaired in the donor, and F_{REPAIR} is a number of TCRβ genes identified as repaired TCRβ genes in both the recipient and the donor.

A PSF_{DONOR-REPAIR} score equal to or greater than 0.69 can be indicative of a compatibility between the donor and the recipient. Alternatively, a PSF_{DONOR-REPAIR} score less than 0.69 can be indicative of an incompatibility between the donor and the recipient, and a likelihood of the recipient to develop cGvHD. For example, a PSF_{DONOR-REPAIR} score less than the range of about 0.69 to 0.3 can be used to predict cGvHD. (See FIG. 13A)

A favorable score can be defined as a score that would be interpreted, by a physician or another health care professional responsible for assessing the risk of developing GvHD in a recipient, as in favor of a transplant of the bone marrow or hematopoietic stem cell transplant from the donor to the recipient. An unfavorable score can be defined as a score that would be interpreted as not in favor of the transplant of the bone marrow or a hematopoietic stem cell transplant from the donor to the recipient. The method described herein can further include the treatment of the recipient, which generally comprises the transplant of bone marrow or a hematopoietic stem cell transplant from the donor to the recipient. As described herein, the treatment is to be administered to the recipient, when the score determined by the method described herein is favorable.

Method of Predicting Cancer Relapse in a Subject

Any new screening method for reducing GvHD risk could inadvertently increase cancer relapse risk because GvHD is associated with an anti-cancer response. However, both GvHD and cancer relapse can be avoided, suggesting GvHD screenings accompanied with cancer relapse screenings could be used to minimize the risks for both outcomes. Because no TCR repertoire has specificity for every antigen, it is hypothesized that the recipient's cancer takes advantage of any gaps in the recipient's TCR specificities. According to this hypothesis, a donor with lots of TCRs different from the recipient will be more likely to fill these gaps than a donor with the same TCRs as the recipient. Herein, it was demonstrated that the quantification of donor TCRs not in the recipient can be utilized as a marker for predicting cancer relapse, which is separate from our marker for predicting GvHD.

Methods of predicting cancer relapse in a subject are provided.

The methods can comprise classifying TCRβ genes of a hematopoietic stem cell donor and TCRβ genes of a hematopoietic stem cell recipient as productive TCRβ genes or repaired TCRβ genes using the methods described here; comparing a number of repaired TCRβ genes in both the hematopoietic stem cell donor and the hematopoietic stem cell recipient; and quantifying a number of repaired TCRβ genes that in the hematopoietic stem cell donor that are not found in the hematopoietic stem cell recipient, thereby predicting cancer relapse.

The hematopoietic stem cell recipient can be a subject having cancer. Repaired TCRβ genes from the hematopoietic stem cell donor that are absent in the hematopoietic stem cell recipient can be likely to produce a T cell receptor (TCR) that recognizes cancer cells in the hematopoietic stem cell recipient.

Quantifying can comprise calculating a f_{NOVEL} score, wherein the f_{NOVEL} score is the fraction of the total number of TCRβ genes identified as repaired TCRβ genes in the hematopoietic stem cell donor excluding the number of repaired TCRβ genes that are in common between the hematopoietic stem cell recipient and the hematopoietic stem cell donor.

The lower the f_{NOVEL} score between the hematopoietic stem cell recipient and the hematopoietic stem cell donor is, the higher the risk of cancer relapse can be.

The higher the f_{NOVEL} score between the hematopoietic stem cell recipient and the hematopoietic stem cell donor is, the higher the chance of an absence of cancer relapse can be. The TCRβ gene sequence can comprise a CDR3 sequence of the TCRβ gene. The first three amino acids and the last three amino acids of the CDR3 sequences from the TCRβ gene sequence can be removed.

A f_{NOVEL} score equal to or greater than 0.994 is indicative of a likelihood of the TCRβ genes from the donor to produce TCRβ that recognizes cancer cells, and a likelihood that of the recipient not to develop cancer relapse. Alternatively, a f_{NOVEL} score lesser than 0.994 is indicative of an absence of likelihood of the TCRβ genes from the donor to produce TCRβ that recognizes cancer cells, and a likelihood that of the recipient develops cancer relapse.

The cancer can be selected from the group consisting of leukemias, lymphomas, and hematologic malignancies.

A favorable score can be defined as a score that would be interpreted, by a physician or another health care professional responsible for assessing the risk of cancer relapse in an hematopoietic stem cell recipient, as in favor of a transplant of the bone marrow or hematopoietic stem cell transplant from the donor to the recipient. An unfavorable score can be defined as a score that would be interpreted as not in favor of the transplant of the bone marrow or a hematopoietic stem cell transplant from the donor to the recipient. The method described herein can further include the treatment of the recipient, which generally comprises the transplant of bone marrow or a hematopoietic stem cell transplant from the donor to the recipient having cancer. As described herein, the treatment is to be administered to the recipient, when the score determined by the method described herein is favorable.

Machine Learning System

The machine learning system describes herein can be applied to any immune cell receptor. For example, the immune receptor chain gene can be selected from the group consisting of T cell receptor (TCR), TCR alpha chain (TCRα), TCR beta chain (TCRβ), TCR delta chain (TCRβ), TCR gamma chain (TCRγ), B cell receptor (BCR), BCR light chain (BCRL), BCR heavy chain (BCRH), immunoglobulin light chain (IgL), immunoglobulin heavy chain (IgH), immunoglobulin kappa chain (Igκ) and immunoglobulin lambda chain (Igλ). Exemplified herein is a machine leaning system using TCRβ gene as the immune receptor chain gene. The gene segments can be selected from the group consisting of variable (V) gene segments, diversity (D) gene segments, joining (J) gene segments, and any combination thereof.

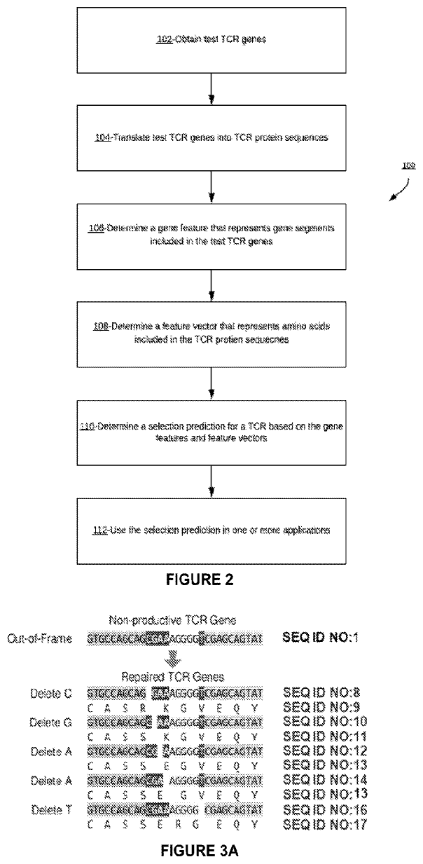

FIG. 2 illustrates an exemplary process for predicting T cell selection 100. At step 102, a set of test TCRβ genes is obtained from a tissue sample. For example, the set of test TCRβ genes may be sequenced from developing T cells included in tissue from the thymus to obtain TCRβ genes that have not undergone T cell selection. The set of test TCRβ genes may also be obtained from mature T cells included in other peripheral tissues (e.g., from the spleen, colon, skin, and the like). The set of test TCRβ genes may include productive TCRβ genes that express a functioning TCRβ included in a mature T cell. The set of test TCRβ genes may also include non-productive TCRβ genes that are unable to express a functioning TCR. At step 104, the set of test TCRβ genes are translated into TCRβ protein sequences. To translate, the sequences of the non-productive TCRβ genes into protein sequences, the non-productive TCRβ genes may be repaired using one or more algorithms to transform the non-productive TCRβ genes into production TCRβ genes.

To maximally preserve the original biological sequences, which contain the intricate and complex biases of natural TCRβ gene recombination, the computer algorithms repair each non-productive TCRβ gene using the fewest alterations required to obtain a productive copy. The repaired TCRβ genes more closely mimic the gene alterations that occur naturally. Therefore, analyzing the surgically repaired TCRβ genes improves the accuracy and precision of methods for reconstituting T cell selection over other techniques including methods that rely on simulating the recombination gene segments (e.g., V, D, and J segments) to create simulated TCRβ genes.

Each repair may be weighted according to the probability of that repair appearing naturally among the subject's non-productive genes. To determine the weight (i.e., the probability that repair occurs naturally) for each repair, the subsequence of the TCRβ gene may be isolated around the repair and the probability of observing that subsequence in non-productive TCRβ genes of the subject may be used to determine the weight of the repair. To weight the repairs based on the occurrence of the subsequence around the repair in subject's non-productive TCRβ genes, the subsequence around the repair may be isolated by defining a radius around the repair (i.e., two nucleotides) and including every nucleotide within this radius in the subsequence. An additional symbol paired with each nucleotide indicating the gene segment (i.e., V(D)J) annotations of the nucleotide may also be included. For example, a nucleotide could be paired with V to indicate the nucleotide is from a V-segment, D to indicate the nucleotide is from a D-segment, J to indicate the nucleotide is from a J-segment, or S to indicate the nucleotide is from a somatic alteration. Every subsequence may then be isolated from every non-productive TCRβ gene of the subject. Using the same radius as before, a radius around every position in a somatic junction may be defined and every nucleotide within this radius may be included in the subsequence. This operation may be performed for every position in a somatic junction for every non-productive TCRβ gene to isolate all relevant subsequences of the subject. The probability of observing the subsequence around the repair may then be calculated by dividing the number of times the subsequence is isolated among non-productive TCRβ genes by the number of subsequences isolated among all of the subject's the non-productive TCRβ genes. This value may then be used as the probability for determining the weight of the repair.

FIG. 3 illustrates exemplary alterations that are made to repair the non-productive TCRβ genes. As shown in section “a” at the top of FIG. 3, for cases where the V and J gene segments are in different open reading frames, the algorithms walk through every permutation for removing the minimal number of nucleotides from the somatic junctions to bring the gene segments into the same open reading frame. Depending on the reading frames, the algorithms remove only one or two nucleotides. As shown in section “b” at the bottom of FIG. 3, for cases where there is a stop codon, the algorithms walk through every possible nucleotide mutation in the somatic junction that converts the stop codon to an amino acid residue. To remove a stop codon, the algorithms mutate only one nucleotide. There are many ways to repair non-productive TCRβ genes, therefore, multiple repaired TCRβ genes are generated from a single non-productive copy. Repairing non-productive TCRβ genes can comprise removing a nucleotide at a somatic junction between gene segments to bring the gene segments in a same reading frame or mutating a nucleotide in a somatic region between gene segments to convert a stop codon into an amino acid. Repairing an TCRβ gene identified as non-productive can comprise generating a repaired TCRβ gene.

The protein sequences of TCRβs that survived T cell selection may be obtained by translating the productive TCRβ genes. To approximate the full pre-selection library of TCRβs, the protein sequences of TCRβs that were not subjected to T cell selection may be obtained by translating the repaired TCR genes. Both types of TCRβ genes may be simultaneously captured by bulk TCRβ sequencing, which can provide upwards of 10⁵ distinct TCRβ genes from a single run, with at least 80% of TCR genes typically being productive (assuming β-chain).

At step 106, gene features are determined for the TCRβ genes. The gene features represent the TCRβ genes in a machine-readable format that may be interpreted by a machine learning system. To generate the gene features, the gene segments included in each TCRβ gene may be input into an encoding layer that outputs one or more gene features that transfer the meaning included in the genetic code of each gene segment into a quantitative format (e.g., a number that describes a position in a multi-dimensional vector space). At step 108, feature vectors are determined for the TCRβ protein sequences. The feature vectors represent the TCRβ protein sequences in a machine-readable format that may be interpreted by a machine learning system. To generate the feature vectors, the TCRβ protein sequences may be input into a encoding layer that outputs one or more protein features that transfer the meaning included in the amino acid sequence of each TCRβ protein into a quantitative format (e.g., a number that describes a position in a multi-dimensional vector space).

At step 110, the machine learning system determines a selection prediction for each TCR included in the set of TCRβ genes based on the gene features and the protein features. The machine learning system may generate a selection prediction by determining the probability that the TCRβ is from a productive TCRβ gene or a non-productive, repaired TCRβ gene. A non-productive TCRβ gene can be a TCRβ gene with out-of-frame gene segments or a TCRβ gene with a stop codon in a somatic junction between gene segments. A TCRβ gene encoding an amino acid sequence involving an antigen recognition can be identified as a productive TCRβ gene, and a TCRβ gene with an amino acid sequence not involving an antigen recognition can be identified as a non-productive TCRβ gene. TCRβs having a probability of originating from a productive TCRβ gene that is greater than the probability of originating from a non-productive, repaired TCRβ gene may be predicted to survive T cell selection. TCRβs having a probability of originating from productive TCRβ gene that is less than the probably of originating from a non-productive, repaired TCRβ gene may be predicted to be culled during T cell selection. At step 112, the T cell selection predictions for the TCRβs may be used in one or more applications as described below.

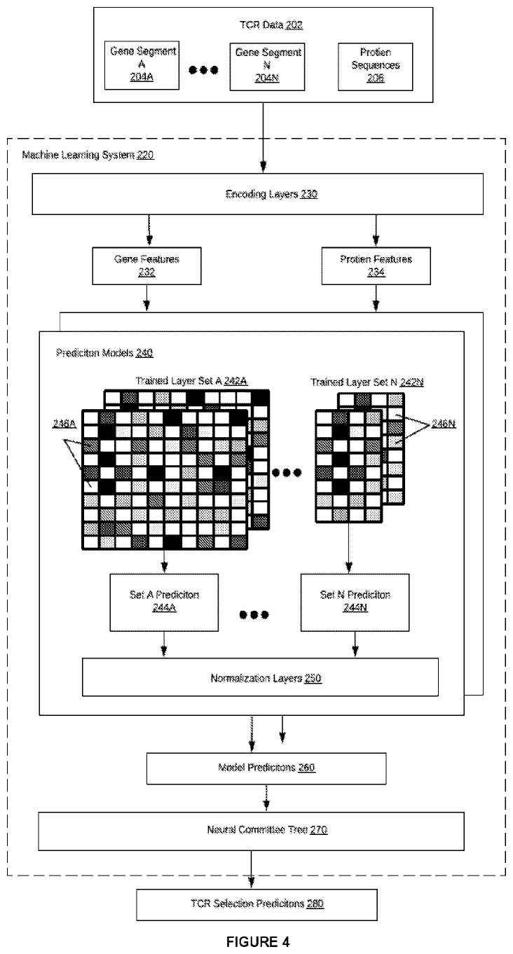

FIG. 4 illustrates an exemplary machine learning system 220. The machine learning system 220 may simulate an immune cell selection process (e.g., T cell selection, B cell selection, and the like). The machine learning system may simulate immune cell selection by receiving immune cell selection data 202 an input and generating immune cell selection predictions 280 as an output. The immune cell selection data 202 may include one or more representations of a cell. For example, to simulate T cell selection, the immune cell selection data 202 may include TCR data that represents a T cell receptor beta chain (TCRβ). The TCRβ representation may include gene segments and other genetic information (e.g., gene segment A 204A, . . . , gene segment N 204N) and protein sequences 206 that may represent other aspects (e.g., a complimentary determining region) of the TCRβ chain. For example, gene segment A 204A may be a V gene segment of the TCRβ, gene segment N 204N may be a J gene segment of the TCRβ, and the protein sequences 206 may be an amino acid sequence that represents the complementary determining region 3 (CDR3) (i.e., the region of the of the TCRβ gene that captures the somatic junctions and D gene segment) of the TCR gene encoding the TCRβ. These three components collectively represent the complete TCRβ chain.

One or more encoding layers 230 included in the machine learning system 220 may be used to convert the genetic information and protein sequences included in the immune cell selection data 202 into a machine-readable format that may be understood by the machine learning system 220. For example, the encoding layers 230 may covert the gene segments 204A, . . . , 204N into gene features 232 and the protein sequences 206 into protein features 234. The encoding layers 230 may determine the gene features 232 using one hot encoding or other techniques for mapping categorical variables to a vector representation that can be provided to a machine learning model. For example, the encoding layers 230 may covert a V gene segment of a TCR gene encoding a TCRβ into 28 binary vectors or other gene features 232. The encoding layers 230 may convert a J gene segment of a TCR gene encoding a TCRβ into 14 binary vectors or other gene features 232.

To determine the protein features 234 for the protein sequences 206, the encoding layers 230 may represent each amino acid included in the protein sequences 206 using Atchley numbers (i.e., a piece of data related to a property of each amino acid). For example, the Atchley numbers may include values that correspond loosely to chemical and or physical properties of each amino acid. The amino acid properties represented by the Atchley numbers may include polarity, one or more secondary structure associations, molecular volume, codon diversity, and or electrostatic charge. For example, the encoding layers 230 may determine vectors containing the five Atchley numbers for each amino acid included in the protein sequences 206 and may replace the amino acids with the appropriate Atchley vectors. Therefore, the protein features 234 provided by the encoding layers 230 may be a sequence of numeric vectors corresponding to the Atchley vectors for each amino acid included in each of the protein sequences 206. The number of amino acids included in the protein sequences 206 is variable so the protein features 234 for each protein sequence 206 may include between 8 and 20 vectors.

For B cell selection predictions, the machine learning system 220 may receive B cell selection data 202 that includes B cell receptor (BCR) data for BCR genes that encode BCR heavy chains (BCRH) sequenced from naïve B cells. Developing B cells edit their DNA by V gene segment CDR3 gene segment and J gene segment recombination to assemble de-novo B cell receptor (BCR) genes. Therefore the length of gene segments 204A, . . . , 204N and protein sequences 206 for the BCR genes may be the same as in the TCRβ representation. Therefore, the encoding layers 230 may generate the same number and type of gene features 232 and protein features 234 when predicting B cells selection as are generated when predicting T cell selection.

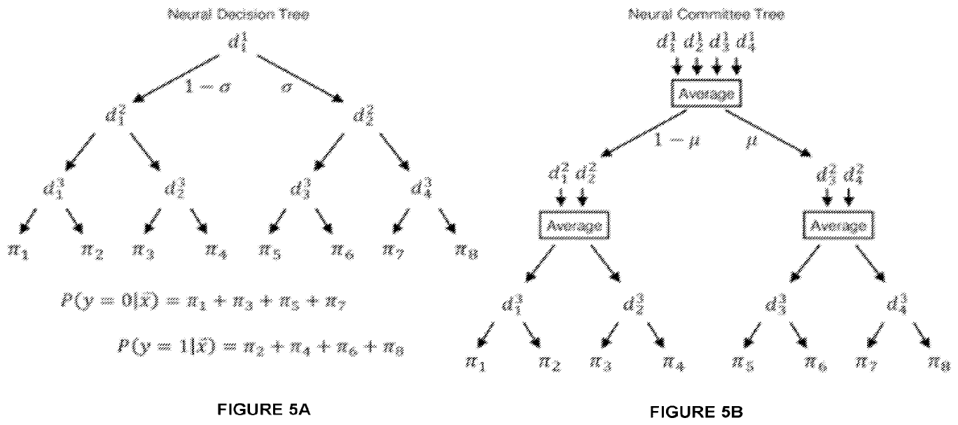

To simulate immune cell section, the gene features 232 and protein features 234 determined by the encoding layers 230 are input into one or more prediction models 240. The prediction models 240 include one or more trained layers (e.g., trained layer set A 242A, . . . , trained layer set N 242N). The gene features 232 and the protein features 234 are multiplied by weight values included in the trained layer sets 242A, . . . 242N to generate set predictions 244A, . . . , 244N. The weight values assigned to each feature may be derived based on a training dataset of prediction specific genes having known selection outcomes. For example, TCR selection predictions may be determined using weight values derived from a training dataset including TCR genes. BCR selection predictions may be determined using weight values derived from a training dataset including BCR genes. The unique weight values for each feature are represented by the different shades included in the squares 246A, . . . , 246N for each trained layer. Each of the squares 246A, . . . 246N included in the trained layer sets 242A, . . . 242N corresponds to one or more of the gene features 232 and or protein features 234 included in the training set of immune cell selection data 202. The optimal weight value to assign to each feature is determined using a training process described below in FIG. 5.

The number of gene features 232 for each of the gene segments 204A, . . . , 204N may be fixed so that the number of weighted values included in the trained layer sets 242A, . . . 242N used to multiply the gene features 232 may be consistent. For example, 28 gene features 232 may be determined for the V gene segment of the TCR gene encoding the TCRβ or the BCR gene encoding the BCRH and 14 gene features 232 may be determined for the J gene segment of the TCRβ gene or BCRH gene. Accordingly, the trained layer sets 242A, . . . , 242N used to handle the gene features 232 may be dense layers having a fixed number of weight values. The number of protein features 234 for each of the protein sequences 206 may be variable because shorter protein sequences may be represented by fewer vectors representing the Atchley numbers for each amino acid. Dynamic kernel matching (also referred to as a dynamic time-alignment kernel) or other techniques for assigning a variable number of features to a prefixed number of weight values may be used to handle the variable number of Atchley number vectors for each protein sequence. For example, the dynamic kernel matching process may require calculating the inner product of the features (i.e., the protein features 234 or other features having a variable number) and weights as a similarity score. An alignment algorithm may then match features and weights to determine an alignment score (i.e., the maximum value for the sum of the similarity scores between the features and the weights). The alignment score is then used to match the variable number of protein features 234 to the fixed number of weights in the trained layers. Each protein feature 234 is then multiplied by its matched weight to generate a prediction.

The set predictions 244A, . . . , 244N generated by each trained layer set 242A, . . . , 242N are then scaled using normalization layers 250 to ensure the expected magnitude for each of the values included in the set predictions 244A, . . . , 244N is the same. For example, the normalization layers 250 may scale the values generated by the trained layers sets 242A, . . . , 242N (i.e., the sum of the products of each gene features 232 and or protein features 234 and its corresponding weight value) so that the expected magnitudes of the set predictions 244A, . . . , 244N for the V gene segment, J gene segment, and the CDR3 are the same. Scaling the values included in the set predictions 244A, . . . , 244N enables the values generated for each of the gene segments 204A, . . . , 204N and protein sequences 206 to be combined to generate a model prediction 260 for the complete TCRβ or BCRH. The model predictions 260 may be re-scaled by the normalization layers 250 so that the values included in the model predictions 260 generated by each of the prediction models have the same magnitude and can be combined.

An ensemble of prediction models 240 may be used to generate immune cell selection predictions 280. For example, 32 different, individually trained models 240 may be used to generate the immune cell selection predictions 280. A neural committee tree 270 may be used to aggregate the model predictions 260 from each of the machine learning models 240 to generate one TCR selection prediction for each TCR gene encoding each TCRβ and or one BCR selection prediction for each BCR gene encoding each BCRH. The neural committee tree 270 may include a modified neural decision tree architecture. The modified neural decision tree architecture may include a hierarchical arrangement of more than two consecutive decisions that are used to aggregate the model predictions 260 to generate immune cell selection prediction 280. For example, the modified neural decision tree architecture may include a hierarchical arrangement of branches with a decision associated with each branch. The decisions made at the branches located on the upper levels of the hierarchical arrangement determine the path through the decision tree and the terminal decisions reached at the end of the decision tree. FIG. 5 below illustrates a simplified modified neural committee tree architecture included in the neural committee tree.

To generate the immune cell selection predictions 280, the model predictions 260 may be used to make decisions in a neural decision tree included in the neural committee tree 270. To make decisions in the neural decision tree, each of the values included in the model predictions 260 may be passed through a sigmoid function or other mathematical function to generate a probability representing a binary decision. The binary decision corresponding to the probability may be used to make a soft decision on a branch in the neural decision tree. This process is repeated until all decisions in the neural decision tree have been made and a prediction for the input model prediction 260 is determined. The selection predictions determined from each of the model predictions 260 generated by all of the prediction models 240 are then aggregated to generate an immune cell selection prediction 280 for the TCRβ and or the BCRH. For example, the selection predictions determined by the neural committee tree 270 for each of the 32 model predictions 260 generated by the 32 prediction models 240 may be averaged to generate the immune cell selection prediction 280.

To enhance the accuracy of the immune cell selection predictions 280, the neural committee tree 270 may include a modified neural decision tree architecture. The neural committee tree 270 structure may include more weights at the base of the neural decision tree to dilute the excepted contribution of the weights at the base of the neural decision tree to match the excepted contribution of the weights at the terminal branches on the neural decision tree. To add more weights at the base of the neural decision tree, each sigmoid near the base of the neural decision tree may be replaced with a committee of sigmoid functions, with each sigmoid function in the committee receiving a distinct output. Adding more sigmoid functions increases the number of weights required to generate the additional outputs required by each sigmoid function. A decision may be reached by the committee of sigmoid functions by averaging the outputs of each sigmoid function included in the committee.

For example, FIG. 5 illustrates an exemplary neural committee tree architecture as compared to a traditional neural decision tree. Section “a” at the left of the figure illustrates a neural decision tree. In the neural decision tree, each decision d is made by a sigmoid function σ. Neural decision trees make soft decisions that encompass a range of possible outcomes based on weights associated with each branch. The right branch of the neural decision tree is used with weight a and the left branch is used with weight 1−σ. When a branch is traversed, the weight associated with that branch is multiplied by the weights from the proceeding branches. The outputs from the terminal decisions correspond to probabilities. The sum of the probabilities on the branches to the right represent the probability that the outcome is 1. The sum of the probabilities on the branches to the left represent the probability that the outcome is 0. This structure biases the weights on the upper branches of the neural decision tree because the weights associated with the upper branches are repeatedly used to determine the probabilities that correspond to each terminal decision. For example, the weight associated with the top branch on the left side of the illustrated tree (1−σ) is used to calculate the probability for all four of the terminal decisions of the left side of the tree (i.e., π₁, π₂, π₃, and π₄). Conversely, the weight associated with the terminal decision on the far left of the tree (i.e., π₁) is used only once to calculate the probability that corresponds to π₁. To increase the accuracy of the predictions generated by the machine learning system 220, the modified neural committee tree 270 architecture balances the contribution of each decision so that decisions at the base of the tree do not contribute more than terminal decisions. For example, the neural committee tree 270 shown in section “b” at the right of FIG. 5, has four terminal decisions. The base (i.e., the top) of the neural committee tree 270 averages together 4 decisions to ensure that the number of decisions remains the same across the depth of the tree. This modification resolves a vanishing gradient problem which halts increases in predictive performance observed for trees having more than two consecutive decisions by smoothing the learning rate across the levels of the tree. Slowing the learning rate down at the base of the tree to ensures decisions at the base of the tree are not learned faster than decisions at the terminal ends. This allows the learning at the terminal decisions to influence the decisions at the base of the tree and vice versa.

Matching the committee sizes to the number of sigmoid functions at each level in the neural decision tree may further increase the performance of the model. For example, if the tree has 32 terminal branches with 32 sigmoid functions (one sigmoid function for each terminal branch) then the committee size at the base of the neural decision tree is picked to be 32. Using the same number of sigmoid functions at each level in the neural decision tree may ensure that each weight can contribute equally to the final prediction. Using the neural committee tree 270 architecture described above provided as much as a 5% increase in the performance of the model relative to traditional neural decision trees. Additionally, the neural committee tree architecture enabled the performance of the model to continuously increase with increasing numbers of consecutive decisions. Therefore, the size of the neural decision trees used in the neural committee tree 270 was increased until the number of weights in the model was approximately equal to the number of labeled datapoints. This provided a significant increase in performance over traditional decision trees which were observed to achieve maximum performance after only five consecutive decisions.

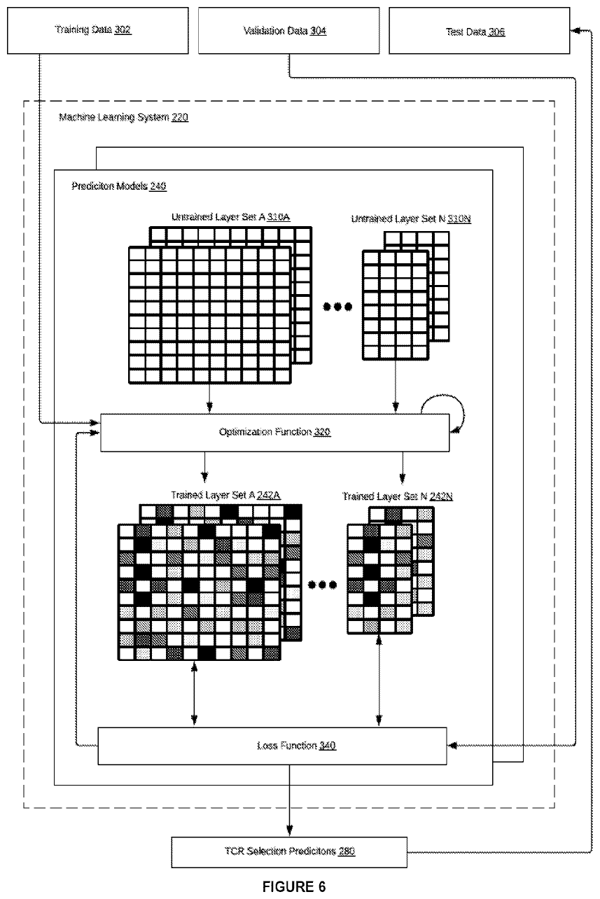

FIG. 6 illustrates an exemplary training process used to determine the weight values included in the trained layer sets 242A, . . . , 242N. To determine the weight values, training data 302 may be used to fit the untrained layer sets 310A, . . . , 310N using an optimization function. The training data 302 may be specific to the type of immune cell selection prediction 280 generated by the machine learning system 220. For example, the training data 302 for T cell selection predictions may include TCR data for TCR genes encoding TCRβs having known selection outcomes. The training data 302 for B cell selection predictions may include BCR data for BCR genes encoding BCRHs having known selection outcomes. To fit the untrained layer sets 310A, . . . , 310N, the weight values include in the untrained layers are randomly initialized. The optimal set of weight values for each feature included in the training data 302 then be determined using a gradient optimization function or other optimization function 320. For example, the gradient optimization function may provide for end-to-end gradient optimization with respect to a loss function. The optimization function 320 may be run through the training data 302 several times (e.g., 128 times) to determine the optimal weight values. Each time through the training data 302, the weight values may be tweaked, and the performance of the model may be tested using validation data 304 (e.g., a data sample that is separate from the training data 302 that includes TCR data and known TCR selection outcomes for T cell predictions, BCR data and known BCR selection outcomes for B cell predictions, and the like).

To test the model performance, the immune cell selection predictions 280 for the TCRβs and of BCRHs included in the validation data 304 (i.e., a 1 for TCRβ chains or BCRH chains from productive genes and a 0 for TCRβ chains or BCRH chains from non-productive and or repaired genes) may be compared to the known selection outcomes. A loss function 340 (e.g., cross-entropy loss function) may measure the error between the selection predictions generated by the model and the known selection outcomes for the TCRβ genes and BCRH genes (i.e., TCR genes encoding the TCRβs and BCR genes encoding the BCRHs respectively) included in the validation set. One or more aspects of the prediction models 240 may be then altered based on the performance of the model. For example, the weight values for gene features and or protein features included in TCRβ genes or BCRH genes that the model was unable to accurately prediction selection for may be tweaked. Training time, learning rate, the number of prediction models used, the number of gene features, and other hyperparameters may also be changed to increase the performance of the model. The weight values and or hyperparameters are tweaked and tested until the minimum error determined by the loss function 340 is achieved for the validation data 304.

The performance of the trained prediction models 240 is then evaluated using test data 306 (i.e., a data sample separate from the training data 302 and validation data 340). The test data 306 may include immune cell selection data that is input into the machine learning system 220 at runtime but has not been previously seen by the prediction models 240 (i.e., has not been used for training and or validation). The prediction models 240 may generate immune cell selection predictions 280 for the TCRβ genes and or the BCRH genes included in the test data 306 using the trained weight values included in the trained layer sets 242A, . . . 242N. The immune cell selection predictions 280 for the TCRβ genes and or the BCRH genes included in the test data 306 may then be compared to the known selection predictions for the TCRβ genes or BCRH genes to determine the performance of the model.

Methods of Use

The machine learning system described herein can be used to predict the risk of developing an autoimmune disease or disorder, the risk of developing alloimmunity from organ transplant, the risk of developing graft-versus-host disease (GvHD) from organ or cellular transplant, the risk of developing alloimmunity from an adoptive T cell therapy, the risk of developing alloimmunity from an chimeric antigen receptor (CAR)-T cell therapy, and to predict the safety of an antibody drug in a subject.

As used herein, a “subject” can be any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

As used herein, the term “predicting a risk of developing” a disease or condition refers to the ability of the methods described herein to indicate with a minimal risk of error, based on a threshold, if a subject is more likely as compared to a healthy subject for example to have or to develop a disease or condition.

Methods of Predicting a Risk of Developing an Autoimmune Disease or Disorder

Methods of predicting a risk of developing an autoimmune disease or disorder in a subject are provided.

The method can comprise reconstituting T cell selection in a matching healthy donor or in multiple healthy donors by classifying each T cell receptors (TCRβ) gene as a productive TCRβ gene or a repaired TCRβ using the machine learning system described herein, applying the T cell selection reconstituted from the donors to the subject, and evaluating a number of escaped T cells in the subject that fail T cell selection in the healthy donor, wherein a number of escaped T cells higher than a threshold indicates a risk of having or of developing an autoimmune disease or disorder.

Predicting a risk of developing an autoimmune disease in a subject can comprise comparing the reconstituted T cells in the subject to the reconstituted T cell in a healthy donor using a sample collected from the subject and a sample collected from the healthy donor.

As used herein, a “sample” or “biological sample” is meant to refer to any “biological specimen” that can be collected from a subject, and that is representative of the content or composition of the source of the sample, considered in its entirety, and that can be used to reconstitute T cell selection in the subject. A sample can be collected and processed directly for analysis or be stored under proper storage conditions to maintain sample quality until analyses are completed. Ideally, a stored sample remains equivalent to a freshly collected specimen. The source of the sample can be an internal organ, vein, artery, or even a fluid. Non-limiting examples of sample include blood, plasma, urine, saliva, sweat, organ biopsy, and cerebrospinal fluid (CSF). In certain embodiments, the sample is peripheral blood or a tissue sample.

As used herein, the term “healthy donor” can include an HLA-matched healthy donor, such as a genetic relative of the subject; the subject himself, or multiple non-HLA-matched healthy donors. A same individual can be the subject and the healthy donor, for example, a sample collected from the individual at a time that is prior the individual is experiencing any symptoms of a disease or condition that can be suspected to be an autoimmune disease, the sample can be used as a sample from a healthy HLA-matched donor, and compared to a sample collected in the individual at a time that is after the individual started experiencing symptoms, at which time the sample collected can be used as a sample from the subject. For example, the sample can be a biospecimen from the subject collected prior to the development of any symptom of a disease, such as banked blood. The biospecimen can also be collected prior to an immune checkpoint inhibitor therapy.

In the absence of an HLA-matched healthy donor, a sample can be collected from multiple healthy donors that are not HLA-matched, and the analysis of the T cell selection can be made by taking into account the HLA status of each healthy donors.

When applying the T cell selection reconstituted from a single healthy donor to a subject, the healthy donor can be an HLA-matched healthy donor. In such case, applying the T cell selection reconstituted from the healthy donor to the subject can comprise sequencing T cell receptors (TCRβ) genes in a sample from the healthy donor, sequencing TCRβ genes in a sample from the subject, and classifying each TCRβ gene as a productive TCRβ gene or a repaired TCRβ gene.

Alternatively, when reconstituting T cell selection in a subject in the absence of an HLA-matched donor available, multiple healthy donors can be used, the multiple healthy donors can be non-HLA-matched healthy donors. In such case, reconstituting T cell selection in multiple healthy donors and applying it to the subject can comprise a) sequencing TCRβ genes in a sample from each donor and in a sample from the subject, b) determining HLA type of each donor and of the subject or sequencing MHC genes for each donor and for the subject, c) tagging each TCRβ gene by the donor's or subject's HLA type, and d) classifying each TCRβ gene as a productive TCRβ gene or a repaired TCRβ gene, using the HLA tag as an additional feature for each TCRβ gene.

Using the machine learning system described herein, reconstituting T cell selection in the donor and applying it to the subject can be used to identify escaped T cells, which are T cells with a productive TCRβ gene misclassified as a repaired TCRβ gene. That is, the system can identify T cells in the subject (i.e., by identifying TCRβ in the subject) that fail T cell selection in the healthy donor, but that pass T cell selection in the subject. T cells that should fail T cell selection are likely to strongly bind self-antigens and therefore to induce an autoimmune reaction in a subject, and a number or T cells that should fail T cell selection (but that are not eliminated) that is above a certain threshold can indicate that the subject has to many T cells that are likely to induce an autoimmune reaction, and is therefore at risk of having or of developing an autoimmune disease or condition.

Autoimmune Diseases and Disorders

The immune system is a system of biological structures and processes within an organism that protects against disease. This system is a diffuse, complex network of interacting cells, cell products, and cell-forming tissues that protects the body from pathogens and other foreign substances, destroys infected and malignant cells, and removes cellular debris: the system includes the thymus, spleen, lymph nodes and lymph tissue, stem cells, white blood cells, antibodies, and lymphokines. B cells or B lymphocytes are a type of lymphocyte in the humoral immunity of the adaptive immune system and are important for immune surveillance. T cells or T lymphocytes are a type of lymphocyte that plays a central role in cell-mediated immunity. There are two major subtypes of T cells: the killer T cell and the helper T cell. In addition, there are suppressor T cells which have a role in modulating immune response. Killer T cells only recognize antigens coupled to Class I MHC molecules, while helper T cells only recognize antigens coupled to Class II MHC molecules. These two mechanisms of antigen presentation reflect the different roles of the two types of T cell. A third minor subtype are the gamma delta T cells (γδ T cells) that recognize intact antigens that are not bound to MHC receptors. γδ T cells are T cells that have a distinctive T-cell receptor (TCR) on their surface. Unlike most T cells that are αβ (alpha beta) T cells with a TCR composed of two glycoprotein chains called α (alpha) and β (beta) TCR chains, γδ T cells have a TCR that is made up of one γ (gamma) chain and one δ (delta) chain. γδ T cells are usually less common than αβ T cells but are at their highest abundance in the gut mucosa, within a population of lymphocytes known as intraepithelial lymphocytes (IELs). The antigenic molecules that activate γδ T cells are largely unknown, and do not seem to require antigen processing and major-histocompatibility-complex (MHC) presentation of peptide epitopes, although some recognize MHC class Ib molecules. γδ T cells are believed to have a prominent role in recognition of lipid antigens. In contrast, the B cell antigen-specific receptor is an antibody molecule on the B cell surface and recognizes whole pathogens without any need for antigen processing. Each lineage of B cell expresses a different antibody, so the complete set of B cell antigen receptors represent all the antibodies that the body can manufacture.

The term “immune response” refers to an integrated bodily response to an antigen and can refer to a cellular immune response or a cellular as well as a humoral immune response. The immune response may be protective/preventive/prophylactic and/or therapeutic.

A “cellular immune response”, a “cellular response”, a “cellular response against an antigen” or a similar term is meant to include a cellular response directed to cells characterized by presentation of an antigen with class I or class II MHC. The cellular response relates to cells called T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells.

The terms “immunoreactive cell” “immune cells” or “immune effector cells” in the context of the present invention relate to a cell which exerts effector functions during an immune reaction. An “immunoreactive cell” can be capable of binding an antigen or a cell characterized by presentation of an antigen, or an antigen peptide derived from an antigen and mediating an immune response. For example, such cells secrete cytokines and/or chemokines, secrete antibodies, recognize cancerous cells, and optionally eliminate such cells. For example, immunoreactive cells comprise T cells (cytotoxic T cells, helper T cells, tumor infiltrating T cells), B cells, natural killer cells, neutrophils, macrophages, and dendritic cells.

As used herein, “autoimmune disorder” or “autoimmune disease” can refer to any medical conditions characterized by a dysfunction of the immune system. Autoimmune diseases are characterized by the abnormal activation and proliferation of self-reactive T- and B-cells, capable of being reactive against substances and tissues normally present in the body (autoimmunity). Self-antigen reactivity can induce damage to or destruction of tissues, alteration of organ growth, and/or alteration of organ function. These disorders can be characterized in several different ways: by the component(s) of the immune system affected; by whether the immune system is overactive or underactive and by whether the condition is congenital or acquired. A major understanding of the underlying pathophysiology of autoimmune diseases has been the application of genome wide association scans that have identified a striking degree of genetic sharing among the autoimmune diseases.

Autoimmune disorders include, but are not limited to, acute disseminated encephalomyelitis (ADEM), Addison's disease, agammaglobulinemia, alopecia areata, amyotrophic lateral sclerosis (aka Lou Gehrig's disease), ankylosing spondylitis, antiphospholipid syndrome, anti-synthetase syndrome, atopic allergy, atopic dermatitis, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune pancreatitis, autoimmune peripheral neuropathy, autoimmune polyendocrine syndrome, autoimmune progesterone dermatitis, autoimmune thrombocytopenic purpura, autoimmune urticaria, autoimmune uveitis, Balo disease/Balo concentric sclerosis, Behcet's disease, Berger's disease, Bickerstaffs encephalitis, Blau syndrome, bullous pemphigoid, cancer, Castleman's disease, celiac disease, chagas disease, chronic inflammatory demyelinating polyneuropathy, chronic inflammatory demyelinating polyneuropathy, chronic obstructive pulmonary disease, chronic recurrent multifocal osteomyelitis, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, dercum's disease, dermatitis herpetiformis, dermatomyositis, diabetes mellitus type 1, diffuse cutaneous systemic sclerosis, discoid lupus erythematosus, Dressler's syndrome, drug-induced lupus, eczema, endometriosis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressiva, fibrosing alveolitis (or idiopathic pulmonary fibrosis), gastritis, gastrointestinal pemphigoid, glomerulonephritis, Goodpasture's syndrome, graft versus host disease, Graves' disease, Guillain-Barré syndrome, Hashimoto's encephalopathy, Hashimoto's thyroiditis, Henoch-Schonlein purpura, herpes gestationis aka gestational pemphigoid, hidradenitis suppurativa, Hughes-Stovin syndrome, hypogammaglobulinemi, idiopathic inflammatory demyelinating diseases, idiopathic pulmonary fibrosis, idiopathic thrombocytopenic purpura, IgA nephropathy, inclusion body myositis, interstitial cystitis, juvenile idiopathic arthritis aka juvenile rheumatoid arthritis, Kawasaki's disease, Lambert-Eaton myasthenic syndrome, leukocytoclastic vasculitis, lichen planus, lichen sclerosus, linear IgA disease, lupoid hepatitis aka autoimmune hepatitis, lupus erythematosus, Majeed syndrome, microscopic colitis, microscopic polyangiitis, Miller-Fisher syndrome, mixed connective tissue disease, Morphea, Mucha-Habermann disease aka pityriasis lichenoides et varioliformis acuta, multiple sclerosis, myasthenia gravis, myositis, Ménière's disease, narcolepsy, neuromyelitis optica, neuromyotonia, ocular cicatricial pemphigoid, opsoclonus myoclonus syndrome, ord's thyroiditis, palindromic rheumatism, PANDAS (pediatric autoimmune neuropsychiatric disorders associated with streptococcus), paraneoplastic cerebellar degeneration, paroxysmal nocturnal hemoglobinuria (PNH), Parry Romberg syndrome, pars planitis, Parsonage-Turner syndrome, pemphigus vulgaris, perivenous encephalomyelitis, pernicious anemia, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pure red cell aplasia, pyoderma gangrenosum, Rasmussen's encephalitis, Raynaud phenomenon, Reiter's syndrome, relapsing polychondritis, restless leg syndrome, retroperitoneal fibrosis, rheumatic fever, rheumatoid arthritis, sarcoidosis, schizophrenia, Schmidt syndrome, Schnitzler syndrome, scleritis, scleroderma, serum sickness, Sjögren's syndrome, spondyloarthropathy, stiff person syndrome, still's disease, subacute bacterial endocarditis (SBE), Susac's syndrome, Sweet's syndrome, sydenham chorea, sympathetic ophthalmia, systemic lupus erythematosus, takayasu's arteritis, temporal arteritis, thrombocytopenia, tolosa-hunt syndrome, transverse myelitis, ulcerative colitis, undifferentiated spondyloarthropathy, urticarial vasculitis, vasculitis, vitiligo, wegener's granulomatosis, myopathies, acne (PAPA), deficiency of the interleukin-1-receptor antagonist (DIRA), allergic reactions, Crohn's disease and Gout.

In certain aspects, the immune disorder is rheumatoid arthritis, systemic lupus erythematosus, celiac disease, Crohn's disease, inflammatory bowel disease, Sjogren's syndrome, polymyalgia rheumatic, psoriasis, multiple sclerosis, ankylosing spondylitis, type 1 diabetes, alopecia areata, vasculitis, temporal arteritis, Graves' disease, or Hashimoto's thyroiditis.

The methods described herein can allow the identification of an autoimmune disease or disorder in a subject. The methods can further comprise, after the identification of such a subject, the administration of a treatment for the autoimmune disease or disorder.

The treatment of autoimmune disorders and diseases can include immunosuppressive and/or anti-inflammatory agents or drugs. The agent may be, for example, an antibody including muromab, basiliximab, and daclizumab, or a nucleic acid encoding one of those antibodies. Examples of immunosuppressive and anti-inflammatory drugs that may be used as the active agent include corticosteroids, rolipram, calphostin, CSAIDs; interleukin-10, glucocorticoids, salicylates, nitric oxide; nuclear translocation inhibitors, such as deoxyspergualin (DSG); non-steroidal anti-inflammatory drugs (NSAIDs) such as ibuprofen, celecoxib and rofecoxib; steroids such as prednisone or dexamethasone; antiviral agents such as abacavir; antiproliferative agents such as methotrexate, leflunomide, FK506 (tacrolimus); cytotoxic drugs such as azathioprine and cyclophosphamide; TNF-α inhibitors such as tenidap, anti-TNF antibodies or soluble TNF receptor, and rapamycin (sirolimus) or derivatives thereof. When the disease is cancer of the thymus, the active agent may be a chemotherapeutic drug or other type of anti-cancer therapeutic.

Methods of Predicting a Risk of Developing Alloimmunity from Organ Transplant

Methods of predicting a risk of developing alloimmunity from organ transplant in an organ recipient are provided.

As used herein, the term “alloimmunity” or “isoimmunity” can refer to an immune response to non-self-antigens from members of the same species (i.e., alloantigens or isoantigens). Two major types of alloantigens are blood group antigens and histocompatibility antigens. In alloimmunity, the body creates antibodies (alloantibodies) against the alloantigens, attacking transfused blood, allotransplanted tissue, and even the fetus in some cases. Alloimmune (isoimmune) response can result for example in graft rejection, which can manifest itself as deterioration or complete loss of graft function. Alloimmunization (isoimmunization) is the process of becoming alloimmune, that is, developing the relevant antibodies for the first time. Alloimmunity can be caused by the difference between products of highly polymorphic genes, primarily genes of the major histocompatibility complex, of a donor and a graft recipient. These products are recognized by T-lymphocytes and other mononuclear leukocytes which infiltrate the graft and damage it.

During organ transplant, an organ is removed from the body of a donor and implanted into the body of an organ recipient to replace a damaged or missing organ. Organs that have been successfully transplanted include the heart, kidneys, liver, lungs, pancreas, intestine, thymus and uterus. Tissues include bones, tendons (both referred to as musculoskeletal grafts), cornea, skin, heart valves, nerves and veins. Organ transplantation is a challenging and complex procedure which requires specific medical management to avoid or manage problems such as transplant rejection, during which the body of the organ recipient can induce an immune response against the transplanted organ, possibly leading to transplant failure and the need to immediately remove the organ from the recipient. When possible, transplant rejection can be reduced through serotyping to determine the most appropriate donor-recipient match and through the use of immunosuppressant drugs.

The method described herein can be used for the prediction of a risk of the organ recipient to generate an immune response against the transplant (alloimmune response). The method can comprise reconstituting T cell selection in an organ donor by classifying each T cell receptors (TCRβ) gene as a productive TCRβ gene or a repaired TCRβ using the machine learning system described herein, applying the T cell selection reconstituted from the donor to the organ recipient, and determining a number of T cells from the organ recipient that are non-tolerant to an organ donor tissue, wherein a number of non-tolerant T cells in the organ recipient higher than a threshold indicates a risk of having or of developing an alloimmunity from e.g., organ transplant.

Reconstituting T cell selection in the organ donor can comprise sequencing TCRβ genes in a sample from the organ donor and classifying each TCRβ gene as a productive TCRβ gene or a repaired TCRβ gene using the machine learning system described herein. A sample collected from the organ donor can be a sample from the transplant. Alternatively, the sample can be is peripheral blood or a sample from another tissue that is not the transplant.

Applying the T cell selection reconstituted from the organ donor to the organ recipient can comprise sequencing TCRβ genes in a sample from the organ recipient and classifying each TCRβ gene as a productive TCRβ gene or a repaired TCRβ gene. A sample from the organ recipient can be peripheral blood or a tissue sample.

Using the machine learning system described herein, reconstituting T cell selection in the donor and applying it to the recipient can be used to identify escaped T cells, which are T cells with a productive TCRβ gene misclassified as a repaired TCRβ gene. That is, the system can identify T cells in the organ recipient (i.e., by identifying TCRβ in the organ recipient) that are predicted to fail T cell selection (i.e., non-tolerant T cell) in the organ donor, but that pass T cell selection in the organ recipient. Non-tolerant T cells that should fail T cell selection in the organ recipient are likely to induce alloimmune reaction in a subject, and a number or T cells that should fail T cell selection (but that are not eliminated) that is above a certain threshold can indicate that the subject has to many T cells that are likely to induce an alloimmune reaction, and is therefore at risk of having or of developing a rejection of the transplanted organ. That is, non-tolerant T cells from the organ recipient are likely to drive an organ transplant rejection.

The methods described herein can allow the identification of an organ recipient that is at risk of developing an alloimmune response after an organ transplant. The methods can further comprise, after the identification of such a subject, the administration of a treatment for the organ rejection or risk thereof.

There is no treatment for hyperacute rejection (which manifests within minutes of the transplant), the only option being the removal of the tissue. Chronic rejection is considered irreversible, with re-transplant being often the best indication for the patients. Acute rejection can be treated with one or more agents.

Despite the use of immunosuppressive therapies, which can include the administration of corticosteroids (such as prednisolone or hypercortisone); calcineutine inhibitors (such as ciclosporin or tacrolimus); anti-proliferative (such as azathioprine or mycophenolic acid); mTOR inhibitors (such as sirolimus or everolimus), antibody-based treatments can be administered. Antibody specific to select immune components can be added to immunosuppressive therapy and can include monoclonal anti-IL-2Rα receptor antibodies (such as basiliximab or daclizumab), polyclonal anti-T-cell antibodies (such as anti-thymocyte globulin (ATG) or anti-lymphocyte globulin (ALG)), monoclonal anti-CD20 antibodies (such as rituximab). Alternatively, blood transfer can be indicated, in cases refractory to immunosuppressive or antibody therapy to remove antibody molecules specific to the transplanted tissue. Marrow transplant can also be used to replace the transplant recipient's immune system with the donors, such that the recipient can accept the new organ without rejection.

Methods of Predicting a Risk of Developing Graft-Versus-Host Disease (GvHD) from Organ or Cellular Transplant

Methods of predicting a risk of developing graft-versus-host disease (GvHD) from organ or cellular transplant in a recipient are provided.

Graft-versus-host disease (GvHD) is a syndrome, characterized by inflammation in different organs, with the specificity of epithelial cell apoptosis and crypt drop out. GvHD is commonly associated with bone marrow transplants and stem cell transplants. GvHD also applies to other forms of transplanted tissues such as solid organ transplants. White blood cells of the donors immune system which can remain within the donated tissue (the graft) can recognize the recipient (the host) as foreign (non-self). The white blood cells present within the transplanted tissue then attack the recipient's body's cells, which leads to GvHD.

The methods described herein can be used for the prediction of a risk of the recipient to develop GvHD from organ or cellular transplant. The methods can comprise reconstituting T cell selection in a recipient by classifying each TCRβ gene as a productive TCRβ gene or a repaired TCRβ gene using the machine learning system described herein, applying the T cell selection reconstituted from the recipient to the donor, and determining a number of T cells from the donor that are non-tolerant to a recipient, wherein a number of non-tolerant T cells in the donor higher than a threshold indicates a risk of having or of developing GvHD from organ or cellular transplant.

Reconstituting T cell selection in the recipient can comprise sequencing TCRβ genes in a sample from the recipient and classifying each TCRβ gene as a productive TCRβ gene or a repaired TCRβ gene using the machine learning system described herein. A sample collected from the recipient can be a sample from the transplant. Alternatively, the sample can be is peripheral blood or a sample from another tissue that is not the transplant.

Applying T cell selection to the donor can comprise sequencing TCRβ genes in a sample from the donor and classifying each TCRβ gene as a productive TCRβ gene or a repaired TCRβ gene using the machine learning system described herein. A sample from the recipient can be peripheral blood or a tissue sample.

Using the machine learning system described herein, reconstituting T cell selection in the recipient and applying it to the donor can be used to identify incompatible T cells, which are T cells with a productive TCRβ gene misclassified as a repaired TCRβ gene. That is, the system can identify T cells in the donor (i.e., by identifying TCRβ in the donor) that are predicted to fail T cell selection in the recipient, but that pass T cell selection in the donor (i.e., non-tolerant T cell). Non-tolerant T cells that should fail T cell selection in the recipient are likely to induce alloimmune reaction in a recipient, and a number or T cells that should fail T cell selection (but that are not eliminated) that is above a certain threshold can indicate that the transplant is likely to comprise too many T cells that are likely to induce an alloimmune reaction, and that the recipient is therefore at risk of having or of developing a GvHD. That is, non-tolerant T cells from the donor are likely to drive a GvHD.

The methods described herein can allow the identification of a recipient that is at risk of developing GvHD after an organ or cellular transplantation. The methods can further comprise, after the identification of such a subject, the administration of a treatment for the GvHD.

Treatment of GvHD can include intravenously administered glucocorticoids, such as prednisone, to suppress the T-cell-mediated immune onslaught on the host tissues. Other substances for GvHD treatment or prophylaxis can include, for example, cyclosporine with methotrexate, sirolimus, pentostatin, etanercept, ibrutinib, and alemtuzumab.

Methods of Predicting a Risk of Developing Alloimmunity from an Adoptive T Cell Therapy

Methods of predicting a risk of developing alloimmunity from an adoptive T cell therapy in a recipient are provided.

As used herein, the term “adoptive T cell therapy,” “engineered TCR therapy,” “TCR T cell therapy” and the like can refer to a cellular immunotherapy that relies on the use of the cells of a subject's or a donor's immune system to eliminate cancer cells. Adoptive T cell therapy involves the isolation and ex vivo expansion of tumor specific T cells to achieve greater number of T cells and the infusion into patients with cancer in an attempt to give their immune system the ability to overwhelm remaining tumor via T cells which can attack and kill cancer cells. There are many forms of adoptive T cell therapy being used for cancer treatment; culturing tumor infiltrating lymphocytes or TIL, isolating and expanding one particular T cell or clone, and even using T cells that have been engineered to potently recognize and attack tumors. The adoptive T cell therapy may be an allogenic CAR T cell therapy or involve allogenic T cells engineered with an additional TCR.

The term “cancer” refers to a group of diseases characterized by abnormal and uncontrolled cell proliferation starting at one site (primary site) with the potential to invade and to spread to other sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof. As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, biliary, colon, rectal, liver, kidney, lung, testicular, breast, ovarian, pancreatic, brain, and head and neck cancers, melanoma, sarcoma, multiple myeloma, leukemia, lymphoma, and the like.

Cancer that begins in blood-forming tissue, such as the bone marrow, or in the cells of the immune system are referred to as hematologic cancer, or blood cancer. Hematologic cancers affect the production and function of blood cells, and are classified in three main types: leukemia, lymphoma, and multiple myeloma.

As used herein, “leukemia” refers to a blood caused by the rapid production of abnormal white blood cells. Examples of leukemia include acute lymphoblastic leukemia (ALL), acute myeloid leukemia (AML), chronic lymphocytic leukemia, chronic myelogenous leukemia, and hairy cell leukemia. As used herein, “lymphoma” refers to a type of blood cancer that affects the lymphatic system. Examples of lymphoma include AIDS-related lymphoma, cutaneous T-cell lymphoma, Hodgkin lymphoma, Hodgkin lymphoma, mycosis fungoides, non-Hodgkin lymphoma, primary central nervous system lymphoma, Sezary syndrome, cutaneous T-Cell lymphoma, and Waldenström macroglobulinemia. As used herein, “myeloma” is a cancer of the plasma cells. Examples of myeloma include chronic myeloproliferative neoplasms, Langerhans cell histiocytosis, multiple myeloma, plasma cell neoplasm, myelodysplastic syndromes, and myelodysplastic/myeloproliferative neoplasms.

The method described herein can be used for the prediction of a risk of developing alloimmunity from an adoptive T cell therapy in a recipient. The method can comprise reconstituting T cell selection in a recipient by classifying each TCRβ gene as a productive TCRβ gene or a repaired TCRβ gene using the machine learning system described herein, applying the T cell selection reconstituted in the recipient to the donor T cells, and determining a number of T cells from the donor that are non-tolerant to the recipient, wherein a number of non-tolerant T cells in the donor higher than a threshold indicates a risk of having or of developing alloimmunity from an adoptive T cell therapy. The donor could be the same person as the recipient or a different person.

Reconstituting T cell selection in the recipient can comprise sequencing TCRβ genes in a sample from the donor and classifying each TCRβ gene as a productive TCRβ gene or a repaired TCRβ gene using the machine learning system described herein. The sample can be peripheral blood, or a tissue sample collected e.g., prior to the ex vivo expansion of the cells.

Applying T cell selection to the donor can comprise sequencing TCRβ genes in a sample from the donor and classifying each TCRβ gene as a productive TCRβ gene or a repaired TCRβ gene using the machine learning system described herein. The sample can be peripheral blood, or a tissue.

Non-tolerant T cells can be T cells with a productive TCRβ gene misclassified as a repaired TCRβ gene. A non-tolerant T cell can be a T cell from the donor that is predicted to fail T cell selection in the recipient. The non-tolerant T cell can be a T cell from the donor that is likely to drive alloimmunity in the recipient. Alloimmunity from an adoptive T cell therapy can comprise unwanted immune attacks from the donor T cells against the recipient's cells and tissues. The sample can be peripheral blood or a tissue sample.

The methods described herein can allow the identification of a recipient that is at risk of developing alloimmunity from an adoptive T cell therapy. The methods can further comprise, after the identification of such a subject, the administration of an anti-cancer treatment.

The term “anti-cancer therapy” or “anti-cancer treatment” as used herein is meant to refer to any treatment that can be used to treat cancer, such as surgery, radiotherapy, chemotherapy, immunotherapy, and checkpoint inhibitor therapy.

Examples of chemotherapy include treatment with a chemotherapeutic, cytotoxic or antineoplastic agents including, but not limited to, (i) anti-microtubules agents comprising vinca alkaloids (vinblastine, vincristine, vinflunine, vindesine, and vinorelbine), taxanes (cabazitaxel, docetaxel, larotaxel, ortataxel, paclitaxel, and tesetaxel), epothilones (ixabepilone), and podophyllotoxin (etoposide and teniposide); (ii) antimetabolite agents comprising anti-folates (aminopterin, methotrexate, pemetrexed, pralatrexate, and raltitrexed), and deoxynucleoside analogues (azacitidine, capecitabine, carmofur, cladribine, clofarabine, cytarabine, decitabine, doxifluridine, floxuridine, fludarabine, fluorouracil, gemcitabine, hydroxycarbamide, mercaptopurine, nelarabine, pentostatin, tegafur, and thioguanine); (iii) topoisomerase inhibitors comprising Topoisomerase I inhibitors (belotecan, camptothecin, cositecan, gimatecan, exatecan, irinotecan, lurtotecan, silatecan, topotecan, and rubitecan) and Topoisomerase II inhibitors (aclarubicin, amrubicin, daunorubicin, doxorubicin, epirubicin, etoposide, idarubicinm, merbarone, mitoxantrone, novobiocin, pirarubicin, teniposide, valrubicin, and zorubicin); (iv) alkylating agents comprising nitrogen mustards (bendamustine, busulfan, chlorambucil, cyclophosphamide, estramustine phosphate, ifosamide, mechlorethamine, melphalan, prednimustine, trofosfamide, and uramustine), nitrosoureas (carmustine (BCNU), fotemustine, lomustine (CCNU), N-Nitroso-N-methylurea (MNU), nimustine, ranimustine semustine (MeCCNU), and streptozotocin), platinum-based (cisplatin, carboplatin, dicycloplatin, nedaplatin, oxaliplatin and satraplatin), aziridines (carboquone, thiotepa, mytomycin, diaziquone (AZQ), triaziquone and triethylenemelamine), alkyl sulfonates (busulfan, mannosulfan, and treosulfan), non-classical alkylating agents (hydrazines, procarbazine, triazenes, hexamethylmelamine, altretamine, mitobronitol, and pipobroman), tetrazines (dacarbazine, mitozolomide and temozolomide); (v) anthracyclines agents comprising doxorubicin and daunorubicin. Derivatives of these compounds include epirubicin and idarubicin; pirarubicin, aclarubicin, and mitoxantrone, bleomycins, mitomycin C, mitoxantrone, and actinomycin; (vi) enzyme inhibitors agents comprising FI inhibitor (Tipifarnib), CDK inhibitors (Abemaciclib, Alvocidib, Palbociclib, Ribociclib, and Seliciclib), Prl inhibitor (Bortezomib, Carfilzomib, and Ixazomib), Phl inhibitor (Anagrelide), IMPDI inhibitor (Tiazofurin), LI inhibitor (Masoprocol), PARP inhibitor (Niraparib, Olaparib, Rucaparib), HDAC inhibitor (Belinostat, Panobinostat, Romidepsin, Vorinostat), and PIKI inhibitor (Idelalisib); (vii) receptor antagonist agent comprising ERA receptor antagonist (Atrasentan), Retinoid X receptor antagonist (Bexarotene), Sex steroid receptor antagonist (Testolactone); (viii) ungrouped agent comprising Amsacrine, Trabectedin, Retinoids (Alitretinoin Tretinoin) Arsenic trioxide, Asparagine depleters (Asparaginase/Pegaspargase), Celecoxib, Demecolcine Elesclomol, Elsamitrucin, Etoglucid, Lonidamine, Lucanthone, Mitoguazone, Mitotane, Oblimersen, Omacetaxine mepesuccinate, and Eribulin.

Method of Predicting Compatibility of an Engineered T Cell Receptor (TCR) Therapy in a Recipient

Method of predicting compatibility of an engineered T cell receptor (TCR) therapy in a recipient are provided.

The methods can comprise reconstituting T cell selection in a recipient by classifying each TCRβ gene as a productive TCRβ gene or a repaired TCRβ gene using the machine learning system described herein, applying the T cell selection reconstituted from the recipient to the engineered TCRβ gene, and determining if the engineered TCRβ is non-tolerant to the recipient.

Reconstituting T cell selection in the recipient can comprise sequencing T cell receptors (TCRβ) genes in a sample from the recipient and classifying each TCRβ gene as a productive TCRβ gene or a repaired TCRβ gene. Applying the T cell selection from the recipient to the engineered TCRβ can comprise classifying the engineered TCRβ gene as a productive TCRβ gene or a repaired TCRβ gene. A non-tolerant engineered TCRβ gene can be a productive TCR gene misclassified as a repaired TCRβ gene. A non-tolerant engineered TCRβ is predicted to fail T cell selection in the recipient. The non-tolerant engineered TCR is likely to drive alloimmunity in the recipient. Alloimmunity from an engineered TCR therapy can comprise unwanted immune attacks from the donor T cells against the recipient's cells and tissues. The sample can be peripheral blood or a tissue sample.

Methods of Predicting a Risk of Developing an Autoimmune Disease or Disorder

Methods of predicting a risk of developing an autoimmune disease or disorder in a subject are provided.

The methods can comprise reconstituting B cell selection in the donors by classifying each B cell receptor (BCR) genes as a productive BCR gene or a repaired BCR gene using the machine learning system described herein and evaluating a number of escaped B cells in the subject, wherein a number of escaped B cells higher than a threshold indicates a risk of having or of developing an autoimmune disease or disorder.

Reconstituting B cell selection in the donors can comprise sequencing B cell receptor (BCR) genes in a sample from the donor. Applying B cell selection reconstituted from the donor to the subject can comprise classifying each BCR gene of the subject as a productive BCR gene or a repaired BCR gene. Escaped B cells can be B cells with a productive BCR gene misclassified as a repaired BCR gene. The sample can be peripheral blood or a tissue sample.

Methods of Predicting an Antibody Drug Safety

Methods of predicting an antibody drug safety in a subject are provided.

As used herein, the term “antibody drug safety” can refer to the toxicity or lack thereof of a drug comprising an antibody. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. There are natural pathways that regulate antibody production in a subject, to ensure that antibodies that would react too strongly with self-antigens can be removed. However, there are no means to predict and anticipate an antibody drug binding to self-antigen in a given subject.

“Antibody,” as used herein, encompasses any polypeptide comprising an antigen-binding site regardless of the source, species of origin, method of production, and characteristics. Antibodies include natural or artificial, mono- or polyvalent antibodies including, but not limited to, polyclonal, monoclonal, multispecific, human, humanized, or chimeric antibodies, single chain antibodies, and antibody fragments. “Antibody fragments” include a portion of an intact antibody, such as the antigen binding or variable region of the intact antibody. Examples of antibody fragments include Fab, Fab′ and F(ab′)2, Fc fragments or Fc-fusion products, single-chain Fvs (scFv), disulfide-linked Fvs (sdfv) and fragments including either a VL or VH domain; diabodies, tribodies and the like (Zapata et al. Protein Eng. 8(10):1057-1062 [1995]).

The term “antibody,” as used herein, refers to immunoglobulin molecules and immunologically active portions of immunoglobulin molecules, i.e., molecules that contain an antigen binding site that immunospecifically binds an antigen. “Native antibodies” and “intact immunoglobulins”, or the like, are usually heterotetrameric glycoproteins of about 150,000 daltons, composed of two identical light (L) chains and two identical heavy (H) chains. The light chains from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: Ig, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known.

The intact antibody may have one or more “effector functions” which refer to those biological activities attributable to the Fc region (a native sequence Fc region or amino acid sequence variant Fc region or any other modified Fc region) of an antibody. Examples of antibody effector functions include Clq binding; complement dependent cytotoxicity; Fc receptor binding; antibody-dependent cell-mediated cytotoxicity (ADCC); phagocytosis; down regulation of cell surface receptors (e.g., B cell receptor (BCR); and cross-presentation of antigens by antigen presenting cells or dendritic cells.

Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains. Each variable region includes three segments called complementarity-determining regions (CDRs) or hypervariable regions and a more highly conserved portions of variable domains are called the framework region (FR). The variable domains of heavy and light chains each includes four FR regions, largely adopting a β-sheet configuration, connected by three CDRs, which form loops connecting, and in some cases forming part of the β-sheet structure. The CDRs in each chain are held together in close proximity by the FRs and, with the CDRs from the other chain, contribute to the formation of the antigen-binding site of antibodies (see Kabat et al., NIH Publ. No. 91-3242, Vol. I, pages 647-669 [1991]). The constant domains are not involved directly in binding an antibody to an antigen, but exhibit various effector functions, such as participation of the antibody in antibody dependent cellular cytotoxicity.

An “antigen” can be any substance that will elicit an immune response. In particular, an “antigen” relates to any substance, such as a peptide or protein, that reacts specifically with antibodies or T-lymphocytes (T cells). The term “antigen” can comprise any molecule that comprises at least one epitope. An antigen in the context of this disclosure is a molecule which, optionally after processing, induces an immune reaction. Any suitable antigen may be used, which is a candidate for an immune reaction, wherein the immune reaction can be a cellular immune reaction. In the context of certain embodiments, the antigen can be presented by a cell by an antigen presenting cell, which includes a diseased cell, in particular a cancer cell, in the context of MHC molecules, which results in an immune reaction against the antigen. An antigen can be a product that corresponds to or is derived from a naturally occurring antigen. Such naturally occurring antigens include tumor antigens.

The term “binding-affinity” generally refers to the strength of the sum total of noncovalent interactions between a single binding site of a molecule (e.g., an antibody), and its binding partner. A variety of methods of measuring binding affinity or binding activity are known in the art, any of which can be used for purposes of the present methods. Specific illustrative embodiments are described in the following.

As used herein, “specific binding” refers to antibody binding to a predetermined antigen. Typically, the antibody binds with an affinity corresponding to a KD of about 10⁻⁸ M or less and binds to the predetermined antigen with an affinity (as expressed by KD) that is at least 10-fold less and can be at least 100-fold less than its affinity for binding to a non-specific antigen (e.g., BSA, casein) other than the predetermined antigen or a closely related antigen. Alternatively, the antibody can bind with an affinity corresponding to a KA of about 10⁶ M⁻¹, or about 10⁷ M⁻¹, or about 10⁸ M⁻¹, or 10⁹ M⁻¹ or higher, and binds to the predetermined antigen with an affinity (as expressed by KA) that is at least 10 fold higher or at least 100 fold higher than its affinity for binding to a non-specific antigen (e.g., BSA, casein) other than the predetermined antigen or a closely-related antigen.

The term “kd” (sec−1), as used herein, is intended to refer to the dissociation rate constant of a particular antibody-antigen interaction. This value is also referred to as the off value. The term “KD” (M⁻¹), as used herein, is intended to refer to the dissociation equilibrium constant of a particular antibody-antigen interaction.

The term “ka” (M⁻¹sec⁻¹), as used herein, is intended to refer to the association rate constant of a particular antibody-antigen interaction. The term “KA” (M), as used herein, is intended to refer to the association equilibrium constant of a particular antibody-antigen interaction.

The methods can comprise reconstituting B cell selection in the subject by classifying each B cell receptor (BCR) gene of the subject as a productive BCR gene or a repaired BCR gene using the machine learning system described herein and determining if a BCR gene encoding the antibody drug is tolerant to subject's self-antigens, wherein a tolerant BCR gene encoding an antibody drug is a BCR gene correctly classified as a productive BCR gene.

Because of the similarities between B and T cells, B cell selection can be reconstituted in-silico using the method for reconstituting T cells. Pertinent differences between B and T cells can include:
Developing B cells edit their DNA by V(D)J recombination to assemble de-novo B cell receptor (BCR) genes (like how T cells assemble TCR genes);
B cell selection occurs in the bone marrow and requires additional steps that take place in the spleen before B cells reach maturity. In contrast, T cells undergo T cell selection in the thymus;
B cells bind antigens independently of MHC molecules because B cells are not required to recognize MHC molecules during B cell selection. In contrast, T cells are required to recognize MHC molecules during T cell selection;
Developing B cells with too high an affinity for self-antigen that fail negative selection are either (i) deleted, (ii) allowed to re-edit their BCR gene, or (iii) placed in an anergic state. B cell selection removes and suppresses B cells that could drive immune attacks against self-antigens on healthy cells and tissue, helping to ensure that the remaining B cells will not drive an autoimmune disease;
Mature B cells that recognize an antigen are sometimes allowed to further edit the DNA of their BCR gene, accumulating additional genetic alterations known as somatic hypermutations (SHMs). SHMs can result in a new BCR with greater affinity for self-antigens;
When reconstituting B cell selection in-silico, naïve B cells can used because naïve B cells have not yet recognized an antigen and therefore have not accumulated SHMs. This allows a focus on B cell selection (e.g., sequencing B cell receptor (BCR) genes in a sample from the subject and classifying each BCR gene of the subject as a productive BCR gene or a repaired BCR gene) without having to take into consideration SHMs. Alternatively, instead of using naïve B cells, all B cells can be used and all BCR sequences that contain SHMS can be removed;
Machine learning is used to discriminate repaired from productive BCR heavy chains (BCRHs) sequenced from naïve B cells from a C57BL/6 mouse. On unique holdout BCRHs representing our test set, the model achieves a balanced classification accuracy of 83.3%. The sensitivity for productive BCRHs is 91.2% and the specificity is 75.3%. A plot of the true positive rate versus the false positive rate for various classification thresholds of our model, known as a receiver operating characteristic (ROC) curve, has an area under the curve (AUC) of 0.91;
Pre-B cells that have not undergone B cell selection and can be classified like repaired BCR genes, confirming that this method can reconstitute some aspects of B cell selection;
B cells can differentiate into plasma cells and produce antibodies, which are essentially BCRs that can detach from the cell and act independently. Because plasma cells originate from B cells, plasma cells undergo B cell selection before becoming plasma cells. A model of B cell selection can be used to determine if an antibody would pass B cell selection. First, a peripheral blood or tissue sample collected from the patient can be sequenced for BCRs. The BCRs can then be used to reconstitute B cell selection in the recipient by fitting a machine learning model to discriminate between repaired and productive BCR genes from the recipient. Next, the BCR encoding the antibody can be passed through the fitted machine learning model generating a prediction. Antibodies classified like repaired receptors would presumably fail B cell selection and may bind self-antigens.

Reconstituting B cell selection in the subject can comprise sequencing BCR genes in a sample from the subject and classifying each BCR gene of the subject as a productive BCR gene or a repaired BCR gene by using the machine learning system described herein.

A non-tolerant BCR gene encoding an antibody drug can be a BCR gene misclassified as a repaired BCR gene. A non-tolerant BCR gene encoding an antibody drug can be a BCR gene that is predicted to fail B cell selection in the subject. The non-tolerant BCR gene encoding an antibody drug can encode an antibody drug that is likely to bind self-antigens in the subject. An antibody drug classified as likely to bind self-antigen can indicate a lack of safety of use of the antibody drug in the subject. The sample can be peripheral blood or a tissue sample.

Methods of Predicting a Risk of Developing Alloimmunity from a Chimeric Antigen Receptor (CAR)-T Cell Therapy

Methods of predicting a risk of developing alloimmunity from a chimeric antigen receptor (CAR)-T cell therapy in a subject are provided.

As used herein, “CAR-T cell therapy” can refer to chimeric antigen receptor T cells (also known as CAR T cells) that have been genetically engineered to produce an artificial T-cell receptor, and that can be used as immunotherapy to treat cancer. Chimeric antigen receptors (CARs, also known as chimeric immunoreceptors, chimeric T cell receptors or artificial T cell receptors) are receptor proteins that have been engineered to give T cells the new ability to target a specific protein. The receptors are chimeric because they combine both antigen-binding and T-cell activating functions into a single receptor. CAR-T cell therapy uses T cells engineered with CARs for cancer therapy. The premise of CAR-T immunotherapy is to modify T cells to recognize cancer cells in order to more effectively target and destroy them. T cells can be harvested from subject, or donors, genetically altered, and infused into patients to attack their tumors. CAR-T cells can be either derived from T cells in a patient's own blood (autologous) or derived from the T cells of another healthy donor (allogeneic). Once isolated from a subject, these T cells are genetically engineered to express a specific CAR, which programs them to target an antigen that is present on the surface of tumors. For safety, CAR-T cells are engineered to be specific to an antigen expressed on a tumor that is not expressed on healthy cells. After CAR-T cells are infused into a patient, they act as a “living drug” against cancer cells. When they come in contact with their targeted antigen on a cell, CAR-T cells bind to it and become activated, then proceed to proliferate and become cytotoxic. CAR-T cells can destroy cells through several mechanisms, including extensive stimulated cell proliferation, increasing the degree to which they are toxic to other living cells (cytotoxicity) and by causing the increased secretion of factors that can affect other cells such as cytokines, interleukins and growth factors.

Methods can comprise determining if an antigen binding domain of the CAR is tolerant to subject's self-antigens, wherein determining if an antigen binding domain of the CAR is tolerant to subject's self-antigens comprises reconstituting B cell selection in the subject by fitting the machine learning system described herein, and determining if a BCR gene encoding the antigen binding domain of the CAR is tolerant to subject's self-antigens, wherein a tolerant BCR gene encoding the antigen binding domain of the CAR is a BCR gene correctly classified as a productive BCR gene.

Reconstituting B cell selection in a subject can comprise sequencing BCR genes in a sample from the subject and classifying each BCR gene of the subject as a productive BCR gene or a repaired BCR gene by using the machine learning system described herein.

A non-tolerant BCR gene encoding the antigen binding domain of the CAR can be a BCR gene misclassified as a repaired BCR gene. A non-tolerant BCR gene encoding the antigen binding domain of the CAR can be a BCR gene that is predicted to fail B cell selection in the subject.

The non-tolerant BCR gene encoding an antibody drug can encode an antibody drug that is likely to bind self-antigens in the subject.

A BCR gene classified as likely to bind self-antigen can indicate a lack of safety of use of the CAR-T cell therapy in the subject.

The sample can be peripheral blood or a tissue sample.

The compositions and methods are more particularly described below, and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1

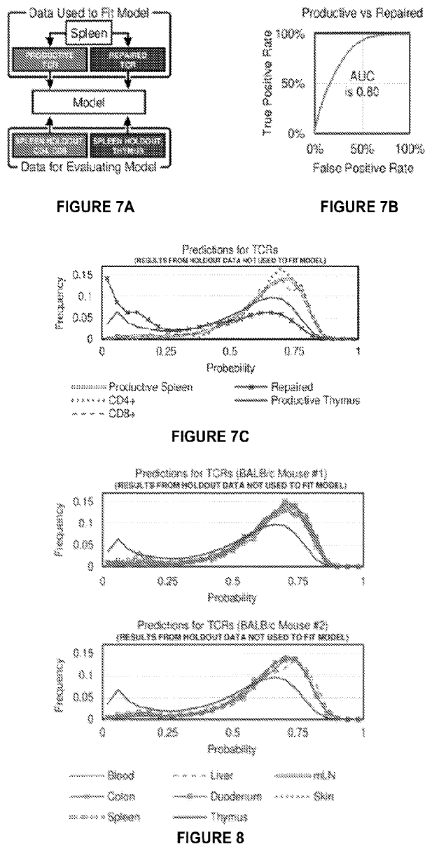

To evaluate the performance of the model, TCR selection was simulated for a single BALB/c mouse. FIG. 7 illustrates the results of the TCR selection simulation. Section “a” in the top right corner of FIG. 7 illustrates the data used to fit and evaluate the model. As shown in Section “a” TCR β-chain (TCRB) genes sequenced from spleen of a single BALB/c mouse are used to fit our machine learning model. This sample of TCRB genes includes both productive TCR genes and non-productive repaired TCR genes. The goal of this analysis is to identify the origin of TCRBs included in the unique holdout test set. The holdout TCRBs include both repaired and productive TCR genes isolated from the Thymus. A test set including a holdout of CD4+ and CD8+ T cells was also used to evaluate the model to see if performance differs between CD4+ and CD8+ T cells.

Model achieved a classification accuracy of 73.2% on the test set evaluated. The sensitivity for TCRBs from productive genes was 85.2% and the specificity for TCRBs from repaired genes was 61.1%. Section “b” at the top right of FIG. 7 illustrates a plot of the true positive rate versus the false positive rate for various classification thresholds of our model. The relationship between the true positive rate and the false positive rate shown in the plot is known as a receiver operating characteristic (ROC) curve and has an area under the curve (AUC) of 0.8. The relatively low specificity for TCRBs from repaired genes in the test set evaluated may be attributable to the fact that repaired genes represent an unselected mix of TCRBs, with some that would survive T cell selection and some that would not. This is reflected in a histogram of the model's predictions for each unique holdout TCRB in our test set shown in section “c” at the bottom of FIG. 7).

The histogram reveals the distribution for the model's predictions for different populations of TCRBs. The histogram of the model's predictions reveals a unimodal distribution for TCRBs from productive genes and a bimodal distribution for TCRBs from repaired genes. The second mode associated with repaired genes corresponds to the single mode associated with productive genes, seeming to represent TCRBs from repaired genes that would survive T cell selection. For TCRBs from productive genes from spleen, a single mode is observed centered at 0.74 (double). The histogram also displays results for particular subsets of T cells (e.g., CD4+ and CD8+ cells). These results demonstrate that the machine learning system may be used to reconstitute T cell selection for T cells isolated from a particular T cell subset. For example, the machine learning system may be used to predict T cell selection results for specific populations of T cells isolated by cell sorting and or T cells isolated by RNA expression.

T cells can be identified by the specific expression of a surface cluster of differentiation (CD) molecule named CD3 and can be separated of two major groups: the CD4 and CD8 populations. The CD4 cells display helper activities on other populations of cells, and can be subdivided into at least Th1, Th2, Th9, Th17 and T regulatory (Treg) groups, each with a characteristic profile of production cytokines. The CD8 T cytotoxic population is the second major group of T lymphocytes that function in killing target cells; they are comprised of Tc1 and Tc2 subpopulations with similar cytokine profiles as Th1 and Th2 cells.

Specific cytokines are involved in shaping the two subsets of the T-cell system: CD4+ T helper (Th) and CD8+ Cytotoxic T Lymphocytes (CTL). That is, aside from the expression of specific CD molecule at their surface (CD4 or CD8), T cells can be differentiated by the cytokines they are producing. For example, Tc1 CD8+ T cells are characterized by their production of TNF-β and IFN-γ; Tc2 CD8+ T cells are characterized by their production of IL-4 and IL-10; Th1 CD4+ T cells are characterized by their production of TNF-β and IFN-γ; Th2 CD4+ T cells are characterized by their production of IL-4, IL-5, IL6, IL-10 and IL-13; Th3 CD4+ T cells are characterized by their production of TGF-β; Th17 CD4+ T cells are characterized by their production of IL-17, IL-21 and IL-22; and Treg CD4+ T cells are characterized by their production of IL-10.

Based on these specific surface protein expressions, T cells from different subsets can be sorted using flow cytometry for example, to differentiate and separate cells based on the protein expressed at their surface. The TCR genes from an isolated subset can be used to reconstitute T cell selection just for that isolated subset. Alternatively, RNA expressions from individual T cells can be used to identify T cells belonging to a specific T cell subset among a population of mixed T cell subsets. For example, expression of genes encoding cytokines can be used to isolate T cells subsets based on intracellular protein expression. By pairing the TCR gene with the RNA expression of each individual T cell, TCR genes belonging to a specific T cell subset can be isolated and used to reconstitute T cell selection just for that T cell subset.

For the subset of CD4+ and CD8+ T cells, both CD4+ and CD8+ T cells are classified like productive TCRB genes from spleen (dotted & dashed). The unimodal distributions for the CD4+ and CD8+ T cells match the unimodal distribution for TCRBs from productive genes collected by bulk sequencing. Therefore, the model successfully classifies T cells regardless if the T cell is CD4+ or CD8+. For TCRBs from repaired genes, a bimodal distribution is observed centered at 0.0 and 0.66 (solid with hashes), with the first mode potentially representing TCRBs that would be culled by T cell selection and the second mode potentially representing TCRBs that would survive T cell selection. TCRBs from thymus show a bimodal distribution like the TCRBs from repaired genes (solid). The distribution for thymic TCRBs is slightly shifted from the distribution associated with repaired genes, because some developing T cells may have partially completed the selection process or mature T cells in thymus are diluting the population of developing T cells. As shown in the histogram, most of the TCRBs from thymic cells are classified like TCRBs from repaired genes.

T cell selection can also be reconstituted using non-regulatory (suppressor) T cells by classifying each TCR gene from non-regulatory T cells as a productive TCR gene or a repaired TCR gene using the machine learning system described herein. Applying the reconstituted T cell selection consists of classifying TCR genes from non-regulatory T cells as either a productive TCR gene or a repaired TCR gene. Removing regulatory T cells ensures T cells escaping negative selection by converting to a regulatory T cell are not used to reconstitute T cell selection or to apply the reconstituted T cell selection.

### Example 2

Sequenced TCRBs from other organs of two mouse individuals including colon and skin, which do not contain developing T cells were also evaluated to determine if the performance of the model is extensible to T cells in other organs. FIG. 8 illustrates histograms of predictions for productive TCRB genes sequenced from blood (solid), colon (solid with x), duodenum (solid with square), liver (dashed), mesenteric lymph nodes (mLN, double), skin (dotted), spleen (double dashed), and thymus (solid). Predictions for the top panel are from the model of BALB/c mouse #1 shown in FIG. 8. Predictions for the bottom panel are from a second mouse individual (a BALB/c mouse #2). The histogram of predictions for these TCRBs reveals a unimodal distribution, almost indistinguishable from the unimodal distribution for TCRBs from productive genes from spleen. Thus, TCRBs sequenced from mature T cells are classified the same way across multiple peripheral tissue sources.

### Example 3

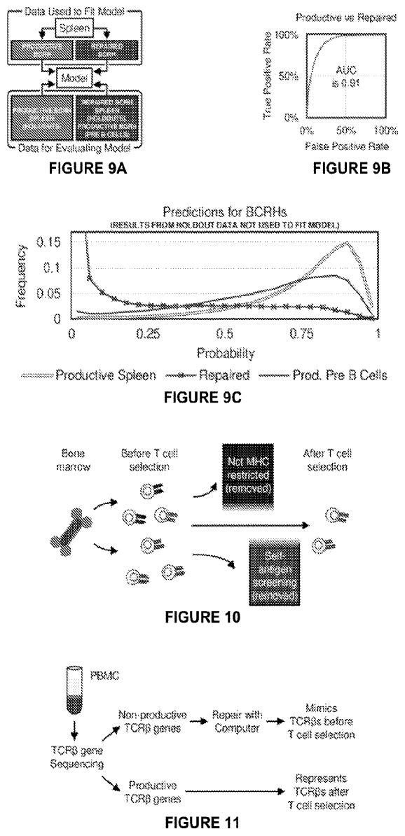

To evaluate the machine learning systems ability to predict B cell selection, a prediction model was fit to distinguish BCRHs from productive and repaired genes from spleen. FIG. 9 illustrates the results of an exemplary B cell selection simulation. The prediction model used to generate the B cell selection predictions is shown in section “a” at the top left of FIG. 9. As shown, a portion of the BCRH obtained from spleen were withheld from the training data set used to fit the model. The withheld sample of BCRHs was used determine the prediction accuracy of the model. For the withheld sample of test data, the model achieves a balanced classification accuracy of 83.3%. The sensitivity for productive BCRHs is 91.2% and the specificity is 75.3%. An ROC curve in section “b” at the top right of FIG. 9 shows the true and false positive rates for different classification thresholds over the holdout data. The area under the ROC curve (AUC) for the predictions on the withheld sample was 0.91. Histograms shown in section “b” at the bottom of FIG. 9 reveal the distribution for the model's predictions for different populations of BCRHs. For BCRHs from productive genes from spleen, a single mode is observed centered at 0.9 (double). For BCRHs from repaired genes, the mode is shifted to the far left (solid with hashes). Productive BCRHs from pre-B cells show a shift from the productive BCR genes from spleen to the repaired BCR genes, indicating the model can identify at least some pre-B cells from the BCR gene (solid).

### Example 4

### Use of Pretransplant T Cell Receptor Sequences to Prognostic GvHD and Cancer Relapse—Approach

Sequenced TCRβ genes from peripheral blood reveal productive and non-productive TCRβ genes that represent the types of TCRβs found before and after T cell selection, respectively. The non-productive TCRβ genes represent the types of TCRβs found before T cell selection because these TCRβ genes never express a receptor for T cell selection, while the productive TCRβ genes are examples of TCRβs found after T cell selection because these TCRβ genes express a receptor that survived T cell selection. Non-productive TCRβ genes from peripheral blood reveal information about the TCRβs removed by T cell selection. However, these comparisons ignored non-productive regions of the TCRβ genes encoding CDR3 that is important for antigen recognition. To include the CDR3 in comparisons, a computer algorithm to computationally repair non-productive TCRβ genes was developed and used, making it possible to compare CDR3s before and after T cell selection (FIG. 11). Briefly, peripheral blood is sequenced for TCRβ genes, non-productive TCRβ genes do not express a receptor chain for T cell selection while productive TCRβ genes express a receptor chain that survives T cell selection. Therefore, repaired non-productive TCRβ genes (the repairing process is described in example 5) represent the types of TCRβs before T cell selection, and the productive TCRβ genes represent TCRβs after T cell selection.

The productive and repaired TCRβs from a recipient are used to infer if donor T cells will be compatible with the recipient. For example, a productive TCRβ from a recipient must have survived T cell selection in the recipient. Therefore, a donor T cell with the same TCRβ could also survive T cell selection in the recipient, indicating the donor T cell would be compatible with the recipient. In a Venn diagram, this is illustrated by the overlap of the donor TCRβs with the recipient's productive TCRβs and is denoted f_{PROD} (see FIGS. 12A and 13A). In contrast, a repaired TCRβ from a recipient might be removed by T cell selection in the recipient. Therefore, a donor T cell with the same TCRβ might also be removed by T cell selection in the recipient, suggesting the donor T cell might be incompatible with the recipient. In a Venn diagram, this is illustrated by the overlap of the donor TCRβs with the recipient's repaired TCRβs and is denoted f_{REPAIR} (see FIGS. 12A and 13A). To quantify the fraction of donor T cells compatible with the recipient, a post-selection fraction, denoted PSFₓ, was developed, where x denotes the sample in the middle of the two Venn diagrams (see FIGS. 12A and 13A).

PSFₓ=f_{PROD}/f_{TOTAL};f_{TOTAL}=f_{REPAIR}+f_{PROD}

The value for PSFₓ calculates the number of compatible donor TCRβs divided by the number of TCRβs in the measurement by comparing the overlap of the top Venn diagram to the sum of the overlaps from both Venn diagrams. A PSFₓ value of 1 predicts all donor TCRβs are compatible with the recipient, while a PSFₓ value of 0 predicts none of the donor TCRβs are compatible with the recipient.

Both the productive and repaired TCRβs from the donor can be screened for compatibility with the recipient. The productive TCRβs represent T cells after T cell selection, like the T cells residing with HSC that are transplanted into the recipient. Therefore, the productive TCRβs from the donor can be screened to determine the compatibility of any transplanted T cells. We determine if the productive TCRβs from the donor contain markers for acute GvHD (aGvHD) because the compatibility of the transplanted T cells is associated with aGvHD. The repaired TCRβs represent T cells before T cell selection, like T cells that develop from donor HSC. Therefore, the repaired TCRβs from the donor can be screened to determine the compatibility of T cells that develop from donor HSC in the recipient. We determine if the repaired TCRβs from the donor contain markers for chronic GvHD (cGvHD) because the compatibility of T cells that develop from donor HSC is associated with cGvHD.

Because T cells are involved in the control of cancer, the TCRβs may contain markers for cancer relapse. The productive TCRβs from the donor represent transplanted T cells that are transient, and thus the transplanted T cells are not expected to be around long-term to prevent cancer relapse. Alternatively, the repaired TCRβs from the donor represent T cells that continuously develop from HSC to replace old T cells, and thus these T cells represent a long-term T cell population that can potentially prevent cancer relapse. Therefore, we evaluate the repaired TCRβs for markers for long-term cancer relapse remission.

### Example 5

### Use of Pretransplant T Cell Receptor Sequences to Prognostic GvHD and Cancer Relapse— Material and Methods

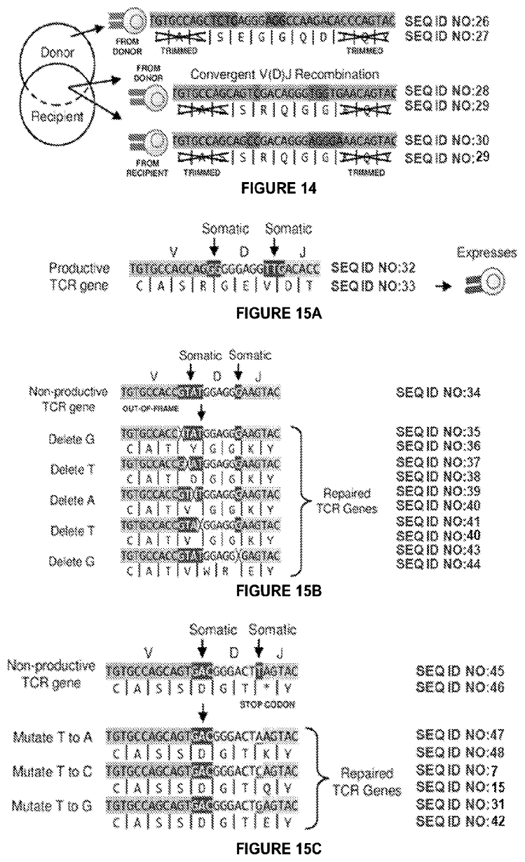

Venn diagram constructions: All Venn diagrams were constructed from the complimentary determining region 3 (CDR3) of each TCRβ because this TCRβ region is involved in antigen recognition. Furthermore, the first and last three amino acid residues from each CDR3 were removed because analyses of 3D X-ray crystallographic structures of TCRβs in contact with antigen revealed the first and last three CDR3 amino acid residues do not directly contact antigen. Based on this insight, donor and recipient TCRβs were considered to be identical when the trimmed CDR3 amino acid sequences were the same, and these TCRβs were placed in the overlapping region of the Venn diagram (see FIG. 14).

Repairing non-productive TCRβ genes: To maximally preserve the original biological sequences, which contain complex and intricate biases from V(D)J recombination, a computer algorithm that surgically repairs each non-productive TCRβ gene using the fewest alterations required to obtain a productive copy was used (see FIG. 15). TCRβ genes can be non-productive because the V and J gene segments are in different open reading frames are referred to as being out-of-frame. When the open reading frame of the J segment is one position ahead of the open reading frame of the V segment, any single nucleotide at a somatic junction was removed to bring the segments into the same open reading frame. When the open reading frame of the J segment is two positions ahead of the open reading frame of the V segment, any two nucleotides at the somatic junctions were removed to bring the segments into the same open reading frame. TCRβ genes can be non-productive because of a stop codon in a somatic junction. These non-productive cases can be identified by translating the TCRβ gene to determine if a stop codon exist in the regions encoded by the somatic junctions. Once a stop codon is identified, the nonproductive TCRβ gene was repaired by mutating any nucleotide in the somatic junction encoding that stop codon to attempt to convert it to an amino acid residue.

All repairs were conducted in somatically encoded junctions because the germline encoded segments are conserved from V(D)J recombination. In some cases, the D gene segment could not be identified after V(D)J recombination because most or all of the D gene segment has been deleted. For this reason, cases where the D gene segment could not be found were not excluded and the nucleotides between the V and J gene segments were treated as a single somatic junction. In many cases, a repaired TCRβ gene will fail to be productive because a new stop codon will be introduced into the TCRβ gene by the repair. Rather than attempt to conduct additional repairs on these TCRβ genes, these cases were discarded out of a concern that multiple repairs will result in TCRβ genes too far away from the original biological sequences to be meaningful. Finally, the repairing algorithm ignored palindromic repeats, potentially breaking these biological patterns when a repair is conducted over a repeat. However, palindromic repeats are present in less than 2% of TCRβ genes allowing us to ignore these infrequent events, as a first approximation.

Template count: The template count is an important number for each sequenced TCRB gene that may reflect the size of the T cell clone. However, the template count is meaningless for non-productive TCRβ genes because these TCRβs cannot express and therefore are not expected to influence clonal expansion. The template count was ignored, effectively treating every productive and repaired TCRβ gene as a singleton.

Sequencing error: TCRβ genes with a large duplicate count are sequenced many times. A handful of these duplicate sequences will inevitably contain sequencing errors. Thus, sufficiently abundant TCRβ genes will contain copies with sequencing errors. This becomes problematic because sequencing error can result in false non-productive TCRβ genes from productive copies. The two types of sequencing error are insertions/deletions and mutations. Sequences where an insertion/deletion or stop codon occurs in germline encoded segments were discarded, reasoning that these alterations should only appear in somatic junctions. All non-productive TCRβ genes that are a single edit distance from a productive TCRβ gene were also discarded, reasoning that sequencing error of the productive copy may have resulted in the non-productive copy.

Statistics: P-values were calculated using a one-sided Mann-Whitney U test assuming a null hypothesis that the cases are at least as high as the controls. Correlation coefficients were calculated using the Pearson correlation coefficient.

### Example 6

### Use of Pretransplant T Cell Receptor Sequences to Prognostic GvHD and Cancer Relapse— Results

Pretransplant TCR β-chain (TCRβ) genes sequenced from 19 allo-HSCT donors and recipients were utilized from two published studies as shown in Table 1. Thirty-two percent of donors were haplotypes (e.g., a parent or mother) while the remaining 68% were matched related donors (MRDs). Sixty-three percent of recipients had acute myeloid leukemia while the rest had other cancer types. Recipients in both studies were monitored for 365 days or death for aGvHD, cGvHD, and cancer relapse. Forty-two percent of recipients developed a GvHD, 47% of recipients developed cGvHD, and 28% of recipients relapsed.

TABLE 1
Clinical characteristics and pretransplant TCRβ repertoires from the donor and recipient for 19
cases have been published. Asterisk (*) denotes patient death during study. Abbreviations are acute
myeloid leukemia (AML), biphenotypic acute leukemia (BAL), myelodysplastic syndrome (MDS), myelofibrosis
(MF), chronic myeloid leukemia (CML), haplotype donor (Haplo), and matched related donor (MRD).

| | | | | | | | | | Number of Unique TCRβs | | | |
|---|---|---|---|---|---|---|---|---|---|---|---|---|
| | Sample | | Type of | | | Acute | Chronic | | Donor | | Recipient | |
| Study | ID | Disease | Donor | Follow Up | | GvHD | GvHD | Relapse | Prod. | Repair | Prod. | Repair |
| JCI | 002-011 | AML | MRD | ≥365 | days | N | N | N | 47605 | 92223 | 39289 | 78791 |
| Insight | 002-016 | AML | MRD | ≥365 | days | Y | N | N | 38541 | 87269 | 29535 | 61004 |
| 2016 | 002-019 | AML | MRD | ≥365 | days | N | N | N | 16282 | 42124 | 62293 | 146875 |
| | 002-037 | AML | MRD | ≥365 | days | Y | N | N | 63938 | 148214 | 3204 | 8050 |
| JCI | CCF1 | AML | MRD | 1764 | days | Y | N | N | 6168 | 14043 | 23031 | 39828 |
| Insight | CCF3 | BAL | Haplo | 997 | days | Y | Y | Y | 45232 | 84942 | 61338 | 107577 |
| 2021 | * CCF5 | MDS/AML | Haplo | * 84 | days | N | | | 2192 | 4633 | 9414 | 17818 |
| | CCF6 | AML | MRD | 1619 | days | Y | N | N | 2785 | 6424 | 7172 | 19079 |
| | CCF9 | AML | MRD | 1441 | days | N | N | N | 15328 | 34777 | 36115 | 71465 |
| | CCF10 | MF | MRD | 1628 | days | N | Y | N | 24440 | 44850 | 913 | 1408 |
| | CCF12 | MF | MRD | 1553 | days | N | Y | N | 88144 | 157530 | 6704 | 9667 |
| | CCF14 | BAL | Haplo | 1606 | days | N | N | N | 51501 | 98442 | 26568 | 50722 |
| | CCF15 | AML | MRD | 1502 | days | Y | Y | N | 15596 | 25867 | 13950 | 26588 |
| | CCF16 | CML | Haplo | 1369 | days | N | Y | Y | 2837 | 5713 | 36802 | 71690 |
| | CCF21 | AML | MRD | 1420 | days | Y | Y | N | 20878 | 32109 | 51356 | 98063 |
| | * CCF25 | AML | Haplo | * 149 | days | N | | Y | 18018 | 31308 | 16010 | 28649 |
| | CCF27 | AML | Haplo | 1336 | days | N | N | Y | 20711 | 36356 | 8680 | 18079 |
| | CCF28 | CML | MRD | 1336 | days | N | Y | Y | 35961 | 58593 | 4147 | 7617 |
| | CCF48 | AML | MRD | 1234 | days | Y | Y | N | 20286 | 42107 | 807 | 1989 |

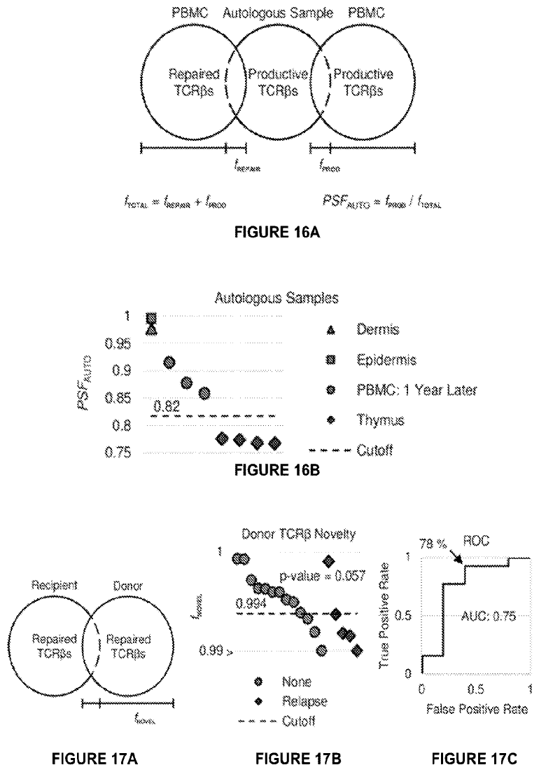

To evaluate productive TCRβs from the donor as a marker for aGvHD, the post-selection fraction of the productive TCRβs from the donor, denoted PSF_{DONOR-PROD}, was calculated to find the fraction of these TCRβs compatible with the recipient (FIG. 12A). A plot of aGvHD cases and controls reveals PSF_{DONOR-PROD} tend to be lower for the aGvHD cases, as anticipated (FIG. 12B). The p-value that PSF_{DONOR-PROD} was lower for aGvHD cases than controls is 0.087. PSF_{DONOR-PROD} below a cutoff of 0.81 was observed for 5/8 aGvHD cases and above this cutoff for 9/11 controls. A cutoff of 0.82 predicts if an autologous sample contains TCRβs before or after T cell selection was observed (FIG. 16), providing a second method for determining this cutoff that achieves essentially the same result. Varying the cutoff for PSF_{DONOR-PROD} yields different true and false positive rates that are plotted in a receiver operating characteristic (ROC) curve (FIG. 12C). The achievable prognostic accuracy (average of the sensitivity and specificity) was 82%. The low prognostic accuracy for aGvHD was attributed to diagnostic uncertainty due to its acute nature and variations in prophylactic treatments that mask the disease.

To evaluate repaired TCRβs from the donor as a marker for cGvHD, the post-selection fraction of the repaired TCRβs from the donor, denoted PSF_{DONOR-PROD}, was calculated to find the fraction of these TCRβs compatible with the recipient (FIG. 13A). A plot of cGvHD cases and controls reveals PSF_{DONOR-REPAIR} tent to be lower for the cGvHD cases, as anticipated (FIG. 13B). The p-value that PSF_{DONOR-REPAIR} was lower for cGvHD cases than controls is 0.019. PSF_{DONOR-REPAIR} was below a cutoff of 0.69 for 8/8 cGvHD cases and above this cutoff for 7/9 controls. The cutoff was lower for cGvHD than aGvHD because the T cells associated with cGvHD undergo impaired T cell selection whereas T cells associated with aGvHD undergo no T cell selection. The achievable prognostic accuracy was 89%, as shown on a ROC curve (FIG. 13C).

To evaluate the repaired TCRβs as a marker for cancer relapse, the fraction of repaired TCRβs from the donor not in the recipient, denoted f_{NOVEL}, was calculated to find the fraction of TCRβs from the donor that recognize antigens the recipient could not, including any cancer antigens (FIG. 17A). A plot of cancer relapse cases and controls revealed that f_{NOVEL} values tent to be lower for the cancer relapse cases, as hypothesized (FIG. 17B). The p-value that f_{NOVEL} was lower for cancer relapse cases than controls was 0.057. f_{NOVEL} was below a cutoff of 0.994 for 4/5 cancer relapse cases and above this cutoff for 10/13 controls. The achievable prognostic accuracy was 78%, as shown on a ROC curve (FIG. 17C).

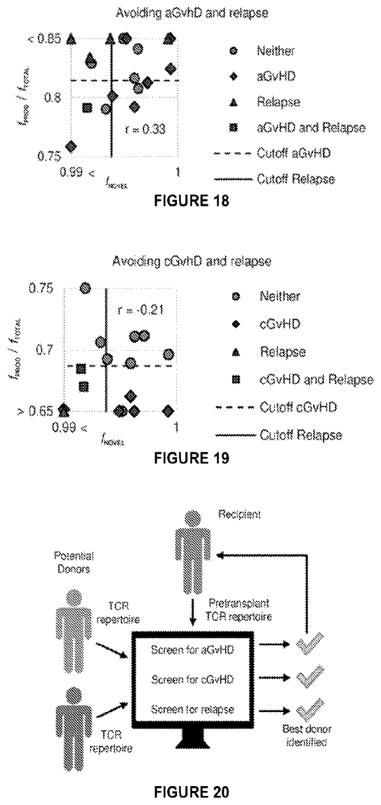

Because GvHD is associated with an anti-cancer response, PSF and f NOVEL were evaluated as joint markers to determine if both GvHD and cancer relapse can be avoided. Because the results for cGvHD were better than aGvHD, combining the marker for cGvHD with the marker for cancer relapse was prioritized (FIGS. 16 and 18). PSF_{DONOR-REPAIR} was plotted against f_{NOVEL} for 17 recipients (FIG. 19). The correlation coefficient for PSF_{DONOR-REPAIR} and f_{NOVEL} was r=−0.21, indicating any correlation was almost non-existent. Cutoffs of 0.69 were used prognosticate cGvHD and cutoffs of 0.994 were used to prognosticate cancer relapse, as previously determined. Recipients above both cutoffs were predicted to avoid both negative outcomes. Cutoffs correctly identify 5/5 recipients that avoid both cGvHD and cancer relapse. Without cutoffs, 8/17 of recipients avoided both cGvHD and cancer relapse.

Samples were initially collected to confirm TCRβ genes can be distinguished before and after T cell selection. TCRβ genes sequenced from peripheral blood from 8 human subjects are shown in Table 2. Four of these subjects have TCRβ genes sequenced from autologous thymic tissue enriched with T cells before T cell selection. The other four subjects have TCRβ genes sequenced from PBMC collected 1 year later or skin that contain T cells after T cell selection.

TABLE 2
TCRβ genes sequenced from PBMC
and autologous samples from 8 subjects.

| | | Autologous | | PBMC | |
|---|---|---|---|---|---|
| | | | Number of | | |
| | Subject | Tissue | Unique TCRs | | |
| Study | ID | Source | Prod. | Prod. | Repair |
| Mol. | Thymus 1 | Thymus | 161696 | 51578 | 138863 |
| Immuno. | Thymus 2 | Thymus | 133093 | 49227 | 132839 |
| 2020 | Thymus 3 | Thymus | 103378 | 72458 | 199755 |
| | Thymus 4 | Thymus | 93012 | 58497 | 168831 |
| Immune | Case 1 | Dermis | 5254 | 14828 | 28843 |
| Access | | Epidermis | 367 | | |
| Immune | Subject01 | PBMC 1 | 278082 | 337151 | 862028 |
| Access | Subject02 | Year Later | 193007 | 215353 | 455556 |
| | Subject03 | | 157764 | 162608 | 384926 |

For each peripheral blood sample, non-productive TCRβ genes that did not express a functional TCRβ were repaired and separated from productive TCRβ genes that could express a functional TCRβ. For the autologous samples, only productive TCRβ genes were used.

Cutoff distinguishes TCRβs before and after T cell selection: PSF_{AUTO} measures whether the autologous sample contains TCRβs matching productive or repaired TCRβs from peripheral blood (FIG. 16). Autologous thymic samples are known to be enriched with the patient's T cells before T cell selection, which is why PSF_{AUTO} for thymic samples is lower. Autologous PBMC collected 1 year later and skin contain T cells after T cell selection, which is why PSF_{AUTO} for these samples is higher. The optimal cutoff to distinguish T cell populations before or after T cell selection was 0.82. This cutoff helped interpret results for aGvHD cases and controls.

Combining cutoffs for aGvHD and cancer relapse: PSF_{DONOR-PROD} was plotted against f_{NOVEL} for 17 recipients (FIG. 18) The correlation coefficient for PSF_{DONOR-PROD} and f_{NOVEL} was r=0.33, indicating any correlation is almost non-existent and in the wrong direction. Cutoffs of 0.81 were used to prognosticate aGvHD and cutoffs of 0.994 were used to prognosticate cancer relapse, as previously determined. Recipients above both cutoffs were predicted to avoid both negative outcomes. The cutoffs correctly identify 3/7=43% transplants that avoid both aGvHD and relapse. Without the cutoffs, 6/17=35% of recipients avoid both aGvHD and relapse.

### Example 7

### Use of Pretransplant T Cell Receptor Sequences to Prognostic GvHD and Cancer Relapse—Discussion

Prognostic markers for aGvHD, cGvHD, and cancer relapse that can be used to reduce the significant morbidities and mortalities associated with allo-HSCT were identified. For example, an alternative donor or specific GvHD prophylactic treatment can be selected when our markers predict GvHD or cancer relapse (FIG. 20). Assuming an alternative donor or treatment always exists, it can be estimated the achievable reduction in disease incidence by calculating the sensitivity of each marker. From the observed results, it was estimated incidence reductions of:
5/8=62.5% for aGvHD,
8/8=100% for cGvHD, and
4/5=100% for cancer relapse

Thus, the prognostic markers can potentially reduce allo-HSCT morbidities and perhaps even the subsequent mortalities.

Multiple factors hinder the predictions from the GvHD markers. For example, some GvHD diagnoses used to select the cutoffs may be inaccurate because the highly variable clinical manifestations associated with the disease can lead to diagnostic uncertainty. Additional samples from future studies will help mitigate this limitation. Also, the markers are based on T cells, but GvHD is sometimes mediated by B cells and other components of the immune system. Applying othisur approach to develop B cell markers can potentially close any performance gaps remaining with T cell markers. Finally, GvHD is influenced by external factors like posttransplant infections, which can trigger GvHD that would have otherwise not occurred. Thus, there are limits to what can be predicted pretransplant.

By prognosticating GvHD and cancer relapse from pretransplant TCR sequences, candidate donors can be screened for these outcomes in the recipient. The predictions, which only compare T cells, can conceivably be used to identify specific T cells associated with these outcomes. Because different types of comparisons are used to predict GvHD and cancer relapse, we can potentially identify T cells that elicit an anti-cancer response without the alloreactive side-effects that cause GvHD. Therefore, this study is not only important for HSCT but also engineered T cell transfer therapies being explored as cancer treatment options.

Although the present invention has been described with reference to specific details of certain embodiments thereof in the above examples, it will be understood that modifications and variations are encompassed within the spirit and scope of the invention. Accordingly, the methods and compositions are limited only by the following claims.

### REFERENCES

J. Styczynski, G. Tridello, L. Koster, S. lacobelli, A. v. Biezen, S. v. d. Werf, M. Mikulska, L. Gil, C. Cordonnier, P. Ljungman, D. Averbuch, S. Cesaro, R. d. I. Camara, H. Baldomero, P. Bader, G. Basak, C. Bonini, R. Duarte, C. Dufour, J. Kuball, A. Lankester, S. Montoto, A. Nagler, J. A. Snowden, N. Kröger, M. Mohty and A. Gratwohl, “Death after hematopoietic stem cell transplantation: changes over calendar year time, infections and associated factors,” Bone Marrow Transplantation, vol. 55, no. 1, pp. 126-136, 2020.
D. L. Cooper, J. Manago, V. Patel, D. Schaar, T. Krimmel, M. K. McGrath, A. Tyno, Y. Lin and R. Strair, “Incorporation of posttransplant cyclophosphamide as part of standard immunoprophylaxis for all allogeneic transplants: a retrospective, single institution study,” Bone Marrow Transplantation, vol. 56, no. 5, pp. 1099-1105, 2021.
J. Bolaños-Meade, R. Reshef, R. Fraser, M. Fei, S. Abhyankar, Z. Al-Kadhimi, A. M. Alousi, J. H. Antin, S. Arai, K. Bickett, Y. B. Chen, L. E. Damon, Y. A. Efebera, N. L. Geller, S. A. Giralt, P. Hari, S. G. Holtan, M. M. Horowitz, D. A. Jacobsohn, R. J. Jones, J. L. Liesveld, B. R. Logan, M. L. MacMillan, M. Mielcarek, P. Noel, J. Pidala, D. L. Porter, I. Pusic, R. Sobecks, S. R. Solomon, D. J. Weisdorf, J. Wu, M. C. Pasquini and J. Koreth, “Three prophylaxis regimens (tacrolimus, mycophenolate mofetil, and cyclophosphamide; tacrolimus, methotrexate, and bortezomib; or tacrolimus, methotrexate, and maraviroc) versus tacrolimus and methotrexate for prevention of graft-versus-host disease with haemopoietic cell transplantation with reduced-intensity conditioning: a randomised phase 2 trial with a non-randomised contemporaneous control group (BMT CTN 1203),” The Lancet Haematology, vol. 6, no. 3, p. 132, 2019.
R. A. M. C. M. Phelan, “Current use and outcome of hematopoietic stem cell transplantation: CIBMTR US summary slides,” Center for International Blood and Marrow Transplant Research, 2020.
N. Ra, P. Ms, S. R, L. G, P. M, A. C, A. Fr, B. Ra, D. Hj and D. K, “Acute graft-versus-host disease: analysis of risk factors after allogeneic marrow transplantation and prophylaxis with cyclosporine and methotrexate,” Blood, vol. 80, no. 7, pp. 1838-1845, 1992.
M. S. Anderson, E. S. Venanzi, L. Klein, Z. Chen, S. P. Berzins, S. J. Turley, H. v. Boehmer, R. Bronson, A. Dierich, C. Benoist and D. Mathis, “Projection of an Immunological Self Shadow Within the Thymus by the Aire Protein,” Science, vol. 298, no. 5597, pp. 1395-1401, 2002.
A. Liston, S. Lesage, J. Wilson, L. Peltonen and C. C. Goodnow, “Aire regulates negative selection of organ-specific T cells,” Nature Immunology, vol. 4, no. 4, pp. 350-354, 2003.
J. Z. L. L. X. L. J. W. J. W. W. Z. J. C. X. Z. Y. T. H. L. a. W. T. Daijing Nie, “Targeted minor histocompatibility antigen typing to estimate,” Bone Marrow Transplantation, 2021.
S. H. Lim, W. N. Patton, S. Jobson, T. A. Gentle, M. Baynham, I. M. Franklin and B. J. Broughton, “Mixed lymphocyte reactions do not predict severity of graft versus host disease (GVHD) in HLA-DR compatible, sibling bone marrow transplants,” Journal of Clinical Pathology, vol. 41, no. 11, pp. 1155-1157, 1988.
O. Ringden, S. Z. Pavletic, C. Anasetti, A. J. Barrett, T. Wang, D. Wang, J. H. Antin, P. D. Bartolomeo, B. J. Bolwell, C. Bredeson, M. S. Cairo, R. P. Gale, V. Gupta, T. Hahn, G. A. Hale, J. Halter, M. Jagasia, M. R. Litzow, F. Locatelli, D. I. Marks, P. L. McCarthy, M. J. Cowan, E. W. Petersdorf, J. A. Russell, G. J. Schiller, H. Schouten, S. Spellman, L. F. Verdonck, J. R. Wingard, M. M. Horowitz and M. Arora, “The graft-versus-leukemia effect using matched unrelated donors is not superior to HLA-identical siblings for hematopoietic stem cell transplantation,” Blood, vol. 113, no. 13, pp. 3110-3118, 2009.
J. Michalek, R. H. Collins, H. P. Durrani, P. Vaclavkova, L. E. Ruff, D. C. Douek and E. S. Vitetta, “Definitive separation of graft-versus-leukemia- and graft-versus-host-specific CD4+ T cells by virtue of their receptor β loci sequences,” Proceedings of the National Academy of Sciences of the United States of America, vol. 100, no. 3, pp. 1180-1184, 2003.
A. R. Datta, A. J. Barrett, Y. Z. Jiang, A. Guimaraes, D. A. Mavroudis, F. v. Rhee, A. A. Gordon and A. Madrigal, “Distinct T cell populations distinguish chronic myeloid leukaemia cells from lymphocytes in the same individual: a model for separating GVHD from GVL reactions,” Bone Marrow Transplantation, vol. 14, no. 4, pp. 517-524, 1994.
J. R. Currier, M. Yassai, M. A. Robinson and J. Gorski, “Molecular defects in TCRBV genes preclude thymic selection and limit the expressed TCR repertoire,” Journal of Immunology, vol. 157, no. 1, pp. 170-175, 1996.
B. J. Manfras, D. Terjung and B. O. Boehm, “Non-productive human TCR β chain genes represent V-D-J diversity before selection upon function: insight into biased usage of TCRBD and TCRBJ genes and diversity of CDR3 region length,” Human Immunology, vol. 60, no. 11, pp. 1090-1100, 1999.
B. Baumann, M. Potash and G. Kohler, “Consequences of frameshift mutations at the immunoglobulin heavy chain locus of the mouse,” The EMBO Journal, vol. 4, no. 2, pp. 351-359, 1985.
S. Li and M. F. Wilkinson, “Nonsense Surveillance in Lymphocytes,” Immunity, vol. 8, no. 2, pp. 135-141, 1998.
H. Li, C. Ye, G. Ji, X. Wu, Z. Xiang, Y. Li and et al., “Recombinatorial Biases and Convergent Recombination Determine Interindividual TCRβ Sharing in Murine Thymocytes,” Journal of Immunology, vol. 189, no. 5, pp. 2404-2413, 2012.
N. Heikkila, R. Vanhanen, D. A. Yohannes, I. Kleino, I. P. Mattila, J. Saramaki and et al., “Human thymic T cell repertoire is imprinted with strong convergence to shared sequences,” Molecular Immunology, vol. 127, pp. 112-123, 2020.
L. M. O. d. Bruin, M. Bosticardo, A. Barbieri, S. G. Lin, J. H. Rowe, P. L. Poliani and et al., “Hypomorphic Rag1 mutations alter the preimmune repertoire at early stages of lymphoid development,” Blood, vol. 132, no. 3, pp. 281-292, 2018.
T. Wu, J. S. Young, H. Johnston, X. Ni, R. Deng, J. Racine, M. Wang, A. Wang, I. Todorov, J. Wang and D. Zeng, “Thymic Damage, Impaired Negative Selection, and Development of Chronic Graft-versus-Host Disease Caused by Donor CD4+ and CD8+ T Cells,” Journal of Immunology, vol. 191, no. 1, pp. 488-499, 2013.
Y. D. H. S. A. K. T. M. H. M. T. a. T. T. Sakoda, “Donor-derived thymic-dependent T cells cause chronic graft-versus-host disease,” Blood, vol. 109, no. 4, pp. 1756-1764, 2007.
C. G. Kanakry, D. G. Coffey, A. M. Towlerton, A. Vulic, B. E. Storer, J. Chou, C. C. Yeung, C. D. Gocke, H. S. Robins, P. V. O'Donnell, L. Luznik and E. H. Warren, “Origin and evolution of the T cell repertoire after posttransplantation cyclophosphamide,” JCI insight, vol. 1, no. 5, 2016.
S. Pagliuca, C. Gurnari, S. Hong, R. Zhao, S. Kongkiatkamon, L. Terkawi, M. Zawit, Y. Guan, H. Awada, A. Kishtagari, C. M. Kerr, T. LaFramboise, B. J. Patel, B. K. Jha, H. E. Carraway, V. Visconte, N. S. Majhail, B. K. Hamilton and J. P. Maciejewski, “Clinical and basic implications of dynamic T cell receptor clonotyping in hematopoietic cell transplantation,” JCI insight, vol. 6, no. 13, 2021.
J. Yu, L. Lal, A. Anderson, M. DuCharme, S. Parasuraman and D. J. Weisdorf, “Healthcare Resource Utilization (HCRU) and Costs Among Patients with Steroid-Resistant (SR) Chronic Graft-Vs-Host Disease (cGVHD) in the United States: A Retrospective Claims Database Analysis,” Biology of Blood and Marrow Transplantation, vol. 25, no. 3, 2019.
M. A. a. J. F. D. Schroeder, “Mouse models of graft-versus-host disease: advances and limitation,” Disease models & mechanisms, vol. 4, no. 3, pp. 318-333, 2011.
J. A. Rath and C. Arber, “Engineering Strategies to Enhance TCR-Based Adoptive T Cell Therapy,” Cells, vol. 9, no. 6, p. 1485, 2020.
Q. Zhao, Y. Jiang, S. Xiang, P. J. Kaboli, J. Shen, Y. Zhao, X. Wu, F. Du, M. Li, C. H. Cho, J. Li, Q. Wen, T. Liu, T. Yi and Z. Xiao, “Engineered TCR-T Cell Immunotherapy in Anticancer Precision Medicine: Pros and Cons,” Frontiers in Immunology, vol. 12, pp. 658753-658753, 2021.
J. Glanville, H. Huang, A. Nau, O. Hatton, L. E. Wagar, F. Rubelt, X. Ji, A. Han, S. M. Krams, C. Pettus, N. Haas, C. S. L. Arlehamn, A. Sette, S. D. Boyd, T. J. Scriba, O. M. Martinez and M. M. Davis, “Identifying specificity groups in the T cell receptor repertoire,” Nature, vol. 547, no. 7661, pp. 94-98, 2017.
J. Ostmeyer, S. Christley, I. T. Toby and L. G. Cowell, “Biophysicochemical Motifs in T-cell Receptor Sequences Distinguish Repertoires from Tumor-Infiltrating Lymphocyte and Adjacent Healthy Tissue,” Cancer Research, vol. 79, no. 7, pp. 1671-1680, 2019.
C. Pannetier, M. Cochet, S. Darche, A. Casrouge, M. Zoller and P. Kourilsky, “The sizes of the CDR3 hypervariable regions of the murine T-cell receptor beta chains vary as a function of the recombined germ-line segments,” Proceedings of the National Academy of Sciences of the United States of America, vol. 90, no. 9, pp. 4319-4323, 1993.
T. Funck, M. B. Barnkob, N. Holm, L. Ohm-Laursen, C. S. Mehlum, S. Möller and et al., “Nucleotide Composition of Human Ig Nontemplated Regions Depends on Trimming of the Flanking Gene Segments, and Terminal Deoxynucleotidyl Transferase Favors Adding Cytosine, Not Guanosine, in Most VDJ Rearrangements,” Journal of Immunology, vol. 201, no. 6, pp. 1765-1774, 2018.
E. Q. Roldan, A. Sottini, A. Bettinardi, A. Albertini, L. Imberti and D. Primi, “Different TCRBV genes generate biased patterns of V-D-J diversity in human T cells,” Immunogenetics, vol. 41, no. 2, pp. 91-100, 1995.
S. K. Srivastava and H. S. Robins, “Palindromic Nucleotide Analysis in Human T Cell Receptor Rearrangements,” PLOS ONE, vol. 7, no. 12, 2012.
S. Christley, W. Scarborough, E. Salinas, W. H. Rounds, I. T. Toby, J. M. Fonner, M. K. Levin, M. Kim, S. A. Mock, C. Jordan, J. Ostmeyer, A. Buntzman, F. Rubelt, M. L. Davila, N. L. Monson, R. H. Scheuermann and L. G. Cowell, “VDJServer: A Cloud-Based Analysis Portal and Data Commons for Immune Repertoire Sequences and Rearrangements,” Frontiers in Immunology, vol. 9, pp. 976-976, 2018.
N. Heikkila, R. Vanhanen, D. A. Yohannes, I. Kleino, I. P. Mattila, J. Saramaki and et al., “Human thymic T cell repertoire is imprinted with strong convergence to shared sequences,” Molecular Immunology, vol. 127, pp. 112-123, 2020.
C. Desmarais, “TCRB Example Different Tissues from the Same Patient,” 08 04 2015. [Online]. Available: https://doi.org/10.21417/B7NP4W.
C. D. R. E. Anna Sherwood, “TCRB Time Course,” 08 04 2015. [Online]. Available: https://doi.org/10.21417/B7J01X.
C. Pannetier, M. Cochet, S. Darche, A. Casrouge, M. Zoller and P. Kourilsky, “The sizes of the CDR3 hypervariable regions of the murine T-cell receptor beta chains vary as a function of the recombined germ-line segments,” Proceedings of the National Academy of Sciences of the United States of America, vol. 90, no. 9, pp. 4319-4323, 1993.
T. Funck, M. B. Barnkob, N. Holm, L. Ohm-Laursen, C. S. Mehlum, S. Möller and et al., “Nucleotide Composition of Human Ig Nontemplated Regions Depends on Trimming of the Flanking Gene Segments, and Terminal Deoxynucleotidyl Transferase Favors Adding Cytosine, Not Guanosine, in Most VDJ Rearrangements,” Journal of Immunology, vol. 201, no. 6, pp. 1765-1774, 2018.
E. Q. Roldan, A. Sottini, A. Bettinardi, A. Albertini, L. Imberti and D. Primi, “Different TCRBV genes generate biased patterns of V-D-J diversity in human T cells,” Immunogenetics, vol. 41, no. 2, pp. 91-100, 1995.
S. K. Srivastava and H. S. Robins, “Palindromic Nucleotide Analysis in Human T Cell Receptor Rearrangements,” PLOS ONE, vol. 7, no. 12, 2012.

What is claimed is:

1. A method of classifying an immune receptor chain gene comprising:
a) obtaining an immune receptor chain gene sequence comprising multiple gene segments and somatic alterations;
b) translating at least one of the multiple gene segments or somatic alterations into an amino acid sequence;
c) identifying an immune receptor chain gene encoding an amino acid sequence capable of antigen recognition as a productive immune receptor chain gene,
d) identifying an immune receptor chain gene without an amino acid sequence capable of antigen recognition as a non-productive immune receptor chain gene,
e) repairing the amino acid sequence of an immune receptor chain gene identified as non-productive to generate a repaired immune receptor chain gene capable of antigen recognition, and
f) classifying the immune receptor chain gene as a productive immune receptor chain gene or as a repaired immune receptor chain gene,
thereby classifying the immune receptor chain gene.

2. The method of claim 1, wherein the gene segments are selected from the group consisting of variable (V) gene segments, diversity (D) gene segments, joining (J) gene segments, and any combination thereof.

3. The method of claim 1, wherein the immune receptor chain gene is selected from the group consisting of T cell receptor (TCR), TCR alpha chain (TCRα), TCR beta chain (TCRβ), TCR delta chain (TCRδ), TCR gamma chain (TCRγ), B cell receptor (BCR), BCR light chain (BCRL), BCR heavy chain (BCRH), immunoglobulin light chain (IgL), immunoglobulin heavy chain (IgH), immunoglobulin kappa chain (Igκ) and immunoglobulin lambda chain (Igλ).

4. The method of claim 3, wherein the immune receptor chain gene is a TCRβ gene.

5. The method of claim 3, wherein the non-productive TCRβ gene is a TCRβ gene with out-of-frame gene segments or a TCRβ gene with a stop codon in a somatic junction between gene segments.

6. The method of claim 3, wherein repairing non-productive TCRβ gene comprises adding or removing one or more nucleotides at a somatic junction between gene segments to bring the gene segments in a same reading frame and/or mutating a nucleotide in a somatic region between gene segments to convert a stop codon into an amino acid.

7. The method of claim 3, wherein the TCRβ gene sequence comprises a complimentary determining region 1 (CDR1) sequence of the TCRβ gene, a CDR2 sequence of the TCRβ gene, a CDR3 sequence of the TCRβ gene, a combination thereof, or a sequence of a complete TCRβ gene.

8. The method of claim 3, wherein the TCRβ gene sequence comprises a CDR3 sequence of the TCRβ gene.

9. The method of claim 8, further comprising removing the first three amino acids and the last three amino acids of the CDR3 sequences from the TCRβ gene sequence.

10. The method of claim 3, wherein obtaining a TCRβ gene sequence comprises sequencing TCRβ genes is a blood sample from a subject.

11. The method of claim 10, wherein the blood sample is a peripheral blood mononucleated cell sample.

12. The method of claim 3, wherein obtaining a TCRβ gene sequence further comprises isolating T cells from a sample.

13. The method of claim 12, wherein isolating T cells is by cell sorting and/or RNA expression.

14. The method of claim 12, wherein T cells are non-regulatory T cells.

15. The method of claim 1, wherein the subject is human.

16. A method of determining an organ donor/organ recipient compatibility comprising:
a) classifying T cell receptor β (TCRβ) genes of the organ donor and TCRβ genes of the organ recipient as productive TCRβ gene or repaired TCRβ gene using the method of any one of claims 4-15;
b) comparing a number of productive and repaired TCRβ genes in a donor to a number of productive TCRβ genes in a recipient; and
c) quantifying the fraction of TCRβ from the organ recipient that are compatible with the organ donor,
thereby determining an organ donor/organ recipient compatibility.

17. The method of claim 16, wherein quantifying is calculating a post selection fraction PSF score.

18. The method of claim 17, wherein the PSF score is a ratio between the number of compatible TCRβ genes from the organ recipient and the total number of TCRβ genes.

19. The method of claim 18, wherein the PSF ranges from 0 to 1.

20. The method of claim 17, wherein the PSF score is a PSF_{RECIPIENT} score, wherein PSF_{RECIPIENT} score is a ratio between F_{PROD} and F_{TOTAL}, wherein F_{TOTAL} is F_{REPAIR}+F_{PROD}, and wherein F_{PROD} is a number of TCRβ genes identified as productive TCRβ genes in both the organ donor and the organ recipient, and F_{REPAIR} is a number of TCRβ genes identified as repaired TCRβ genes in the organ donor and identified as productive TCRβ genes in the organ recipient.

21. The method of claim 19, wherein a PSF_{RECIPIENT} of zero indicates that none the TCRβ genes sequenced in the organ recipient are compatible with the organ donor.

22. The method of claim 19, wherein a PSF_{RECIPIENT} of 1 indicates that all the TCRβ genes sequenced in the organ recipient are compatible with the organ donor.

23. The method of claim 16, wherein the TCRβ gene sequence comprises a CDR3 sequence of the TCRβ gene.

24. The method of claim 23, the first three amino acids and the last three amino acids of the CDR3 sequences from the TCRβ gene sequence are removed.

25. A method of predicting graft versus host disease (GvHD) in an organ or cellular recipient comprising:
a) classifying T cell receptor β (TCRβ) genes of the donor and TCRβ genes of the recipient as productive TCRβ gene or repaired TCRβ gene using the method of any one of claims 4-15;
b) comparing a number of productive and repaired TCRβ genes in the recipient to a number of productive TCRβ genes in the donor; and
c) quantifying the fraction of TCRβ from the donor that are compatible with the recipient,
thereby predicting GvHD in a recipient.

26. The method of claim 25, wherein the GvHD is acute GvHD (aGvHD).

27. The method of claim 25, wherein the organ or cells is bone marrow or a hematopoietic stem cell transplant.

28. The method of claim 26, wherein predicting aGvHD comprises quantifying a number of productive TCRβ gene from the donor that are compatible with the recipient.

29. The method of claim 28, wherein quantifying comprises calculating a post selection fraction PSF_{DONOR-PROD} score, wherein the PSF_{DONOR-PROD} score is a ratio between F_{PROD} and F_{TOTAL}, wherein F_{TOTAL} is F_{REPAIR}+F_{PROD}, and wherein F_{PROD} is a number of TCRβ genes identified as productive TCRβ genes in both the donor and the recipient, and F_{REPAIR} is a number of TCRβ genes identified as repaired TCRβ genes in the recipient and identified as productive TCRβ genes in the donor.

30. The method of claim 29, wherein a PSF_{DONOR-PROD} of zero indicates that none the TCRβ genes sequenced in the donor are compatible with the recipient.

31. The method of claim 29, wherein a PSF_{DONOR-PROD} of 1 indicates that all the TCRβ genes sequenced in the donor are compatible with the recipient.

32. The method of claim 25, wherein the GvHD is chronic GvHD (cGvHD).

33. The method of claim 32, wherein predicting cGvHD comprises quantifying a number of repaired TCRβ gene from the donor that are compatible with the recipient.

34. The method of claim 33, wherein quantifying comprises calculating a post selection fraction score, denoted PSF_{DONOR-REPAIR}, wherein the PSF_{DONOR-REPAIR} score is a ratio between F_{PROD} and F_{TOTAL}, wherein F_{TOTAL} is F_{REPAIR}+F_{PROD}, and wherein F_{PROD} is a number of TCRβ genes identified as productive TCRβ genes in the recipient and identified as repaired in the donor, and F_{REPAIR} is a number of TCRβ genes identified as repaired TCRβ genes in both the recipient and the donor.

35. The method of claim 34, wherein a PSF_{DONOR-REPAIR} of zero indicates that none the TCRβ genes sequenced in the donor are compatible with the recipient.

36. The method of claim 34, wherein a PSF_{DONOR-REPAIR} of 1 indicates that all the TCRβ genes sequenced in the donor are compatible with the recipient.

37. The method of claim 25, wherein the TCRβ gene sequence comprises a CDR3 sequence of the TCRβ gene.

38. The method of claim 37, wherein the first three amino acids and the last three amino acids of the CDR3 sequences from the TCRβ gene sequence are removed.

39. A method of predicting cancer relapse in a hematopoietic stem cell recipient comprising:
a) classifying T cell receptor β (TCRβ) genes of a hematopoietic stem cell donor and TCRβ genes of a hematopoietic stem cell recipient as productive TCRβ gene or repaired TCRβ gene using the method of any one of claims 4-15;
b) comparing a number of repaired TCRβ genes in both the hematopoietic stem cell donor and the hematopoietic stem cell recipient; and
c) quantifying a number of repaired TCRβ genes in the hematopoietic stem cell donor that are not found in the hematopoietic stem cell recipient,
thereby predicting cancer relapse in the hematopoietic stem cell recipient.

40. The method of claim 39, wherein the hematopoietic stem cell recipient is a subject having cancer.

41. The method of claim 39, wherein repaired TCRβ genes from the hematopoietic stem cell donor that are absent in the hematopoietic stem cell recipient are likely to produce a T cell receptor (TCR) that recognizes cancer cells in the hematopoietic stem cell recipient.

42. The method of claim 39, wherein quantifying comprises calculating a f_{NOVEL} score, wherein the f_{NOVEL} score is the fraction of the total number of TCRβ genes identified as repaired TCRβ genes in the hematopoietic stem cell donor excluding the number of repaired TCRβ genes that are in common between the hematopoietic stem cell recipient and the hematopoietic stem cell donor.

43. The method of claim 42, wherein the lower the f_{NOVEL} score between the hematopoietic stem cell recipient and the hematopoietic stem cell donor is, the higher the risk of cancer relapse is.

44. The method of claim 42, wherein the higher the f_{NOVEL} score between the hematopoietic stem cell recipient and the hematopoietic stem cell donor is, the higher the chance of an absence of cancer relapse is.

45. The method of claim 39, wherein the TCRβ gene sequence comprises a CDR3 sequence of the TCRβ gene.

46. The method of claim 45, the first three amino acids and the last three amino acids of the CDR3 sequences from the TCRβ gene sequence are removed.

47. The method of claim 39, wherein the cancer is selected from the group consisting of leukemias, lymphomas, and hematologic malignancies.

48. A method of predicting immune cell selection for an immune cell receptor chain gene comprising:
obtaining a test immune cell receptor chain gene including multiple gene segments;
translating at least one of the multiple gene segments to an immune cell receptor chain protein sequence;
for at least two of the multiple gene segments, determining a gene feature that numerically represents a gene segment;
for each amino acid included in the immune cell receptor chain protein sequence, determining a protein feature that numerically represents one amino acid; and
determining, by a machine learning system, a selection prediction for the test immune cell receptor chain gene based on the gene features for each of the multiple gene segments, the protein features for each of the amino acids in the immune cell receptor chain protein sequence, and a number of weight values included in one or more models of the machine learning system.

49. The method of claim 48, wherein the immune receptor chain gene is selected from the group consisting of T cell receptor (TCR), TCR alpha chain (TCRα), TCR beta chain (TCRβ), TCR delta chain (TCRδ), TCR gamma chain (TCRγ), B cell receptor (BCR), BCR light chain (BCRL), BCR heavy chain (BCRH), immunoglobulin light chain (IgL), immunoglobulin heavy chain (IgH), immunoglobulin kappa chain (Igκ) and immunoglobulin lambda chain (Igλ).

50. The method of claim 49, wherein the immune receptor chain gene is TCRβ gene.

51. The method of claim 48, wherein the gene segments are selected from the group consisting of variable (V) gene segments, diversity (D) gene segments, joining (J) gene segments, and any combination thereof.

52. The method of claim 51, wherein the selection prediction identifies TCRβ gene as a productive TCRβ gene or a repaired TCRβ gene.

53. The method of claim 51, wherein the machine learning system includes an ensemble of multiple prediction models, each prediction model included in the ensemble of multiple prediction models generates a model prediction and the model predictions from each prediction model are combined to determine the selection prediction.

54. The method of claim 53, wherein a modified neural decision tree architecture including a hierarchical arrangement of more than two consecutive decisions is used to aggregate the model predictions into the selection prediction.

55. The method of claim 54, wherein the architecture of the neural decision tree includes a committee of functions, a number of functions included in the committee of functions increasing from the terminal decision in the neural decision tree to base decision on the neural decision tree.

56. The method of claim 51, further comprising obtaining a training dataset including a library of TCRβ genes and the TCRβ protein sequences of the TCRβ genes; and
training the one or more prediction models included in the machine learning system using the training dataset by determining the weight values included in each prediction model using an optimization process.

57. The method of claim 56, wherein the library of TCRβ genes includes multiple productive genes and multiple non-productive genes.

58. The method of claim 57, wherein a non-productive TCRβ gene is a TCRβ gene with out-of-frame gene segments or a TCRβ gene with a stop codon in a somatic junction between gene segments.

59. The method of claim 57, wherein a TCRβ gene encoding an amino acid sequence capable of antigen recognition is identified as a productive TCRβ gene, and wherein a TCRβ gene without an amino acid sequence capable of antigen recognition is identified as a non-productive TCRβ gene.

60. The method of claim 57, further comprising repairing each of the multiple non-productive genes; and translating each of the repaired non-productive genes into a TCRβ protein sequence.

61. The method of claim 60, wherein repairing non-productive TCRβ gene comprises adding or removing one or more nucleotides at a somatic junction between gene segments to bring the gene segments in a same reading frame and/or mutating a nucleotide in a somatic region between gene segments to convert a stop codon into an amino acid.

62. The method of claim 60, wherein repairing an TCRβ gene identified as non-productive comprises generating a repaired TCRβ gene.

63. The method of claim 56, wherein the library of TCRβ genes and TCRβ protein sequences are obtained from a sample provided by an HLA-matched healthy donor.

64. The method of claim 63, wherein the sample is peripheral blood or a tissue sample.

65. The method of claim 48, wherein the protein feature includes a piece of data related to a property of an amino acid, the property is at least one of a polarity, one or more secondary structure associations, a molecular volume, a codon diversity, or an electrostatic charge.

66. The method of claim 48, wherein only T cells isolated from a particular T cell subset are used.

67. The method of claim 66, wherein the T cells are isolated by cell sorting.

68. The method of claim 66, wherein the T cells are isolated by RNA expression.

69. The method of claim 48, wherein the subject is human.

70. The method of claim 60, wherein each of the repaired non-productive genes is weighted according to a probability that a repair used to generate a particular repaired non-productive gene appears naturally among the subject's non-productive genes.

71. The method of claim 50, wherein the TCRβ gene is from non-regulatory T cells.

72. A method of predicting a risk of developing an autoimmune disease or disorder in a subject comprising:
a) reconstituting T cell selection in a matching healthy donor by classifying each T cell receptor (TCRβ) gene as a productive TCRβ gene or a repaired TCRβ using the machine learning system of any one of claims 45-66,
b) applying the T cell selection reconstituted from the healthy donor to T cells from the subject, and
c) evaluating a number of escaped T cells in the subject that fail T cell selection in the healthy donor,
wherein a number of escaped T cells higher than a threshold indicates a risk of having or of developing an autoimmune disease or disorder,
thereby predicting a risk of developing an autoimmune disease or disorder in the subject.

73. The method of claim 72, wherein reconstituting T cell selection in the healthy donor comprises sequencing TCRβ genes in a sample from the matching healthy donor and classifying each TCRβ gene as a productive TCRβ gene or a repaired TCRβ gene.

74. The method of claim 73, wherein applying T cell selection in the subject comprises sequencing TCR genes in a sample from the subject and classifying each TCRβ gene of the subject as a productive TCRβ gene or a repaired TCRβ gene.

75. The method of claim 72, wherein a healthy donor is an HLA-matched healthy donor.

76. The method of claim 75, wherein the HLA-matched healthy donor is a genetic relative of the subject.

77. A method of predicting a risk of developing an autoimmune disease or disorder in a subject comprising:
a) reconstituting T cell selection in multiple healthy donors by classifying each T cell receptor (TCRβ) gene as a productive TCRβ gene or a repaired TCRβ using the machine learning system of any one of claims 45-66,
b) applying the T cell selection reconstituted from the healthy donors to T cells from the subject, and
c) evaluating a number of escaped T cells in the subject that fail T cell selection in the healthy donors,
wherein a number of escaped T cells higher than a threshold indicates a risk of having or of developing an autoimmune disease or disorder,
thereby predicting a risk of developing an autoimmune disease or disorder in the subject.

78. The method of claim 72, wherein reconstituting T cell selection in multiple healthy donors comprises:
a) sequencing T cell receptors (TCRβ) genes in a sample from each donor,
b) determining HLA type of each donor or sequencing MHC genes for each donor,
c) tagging each TCRβ gene by the donor's HLA type, and
d) classifying each TCRβ gene as a productive TCRβ gene or a repaired TCRβ gene, using the HLA tag as an additional feature for each TCRβ gene.

79. The method of claim 72, wherein applying the T cell selection reconstituted from the healthy donors in the subject comprises:
a) sequencing TCRβ genes in a sample from the subject,
b) determining HLA type of the subject or sequencing MHC genes of the subject,
c) tagging each TCRβ gene by the subject's HLA type, and
d) classifying each TCRβ gene of the subject as a productive TCRβ gene or a repaired TCRβ gene.

80. The method of claim 67 or 72, wherein escaped T cells are T cells with a productive TCR gene misclassified as a repaired TCRβ gene.

81. A method of predicting a risk of developing alloimmunity from organ or cellular transplant in a recipient comprising:
a) reconstituting T cell selection in a donor by classifying each T cell receptors (TCRβ) gene as a productive TCRβ gene or a repaired TCRβ using the machine learning system of any one of claims 45-66,
b) applying the T cell selection reconstituted from the donor to the recipient, and
c) determining a number of T cells from the recipient that are non-tolerant to a donor tissue,
wherein a number of non-tolerant T cells in the recipient higher than a threshold indicates a risk of having or of developing an alloimmunity from organ or cellular transplant, thereby predicting a risk of developing alloimmunity from organ or cellular transplant in the recipient.

82. The method of claim 81, wherein reconstituting T cell selection in the donor comprises sequencing T cell receptors (TCRβ) genes in a sample from the donor and classifying each TCRβ gene as a productive TCRβ gene or a repaired TCRβ gene.

83. The method of claim 81, wherein applying the T cell selection to the recipient comprises sequencing TCRβ genes in a sample from the recipient and classifying each TCRβ gene as a productive TCR gene or a repaired TCRβ gene.

84. The method of claim 81, wherein non-tolerant T cells are T cells with a productive TCR gene misclassified as a repaired TCRβ gene.

85. The method of claim 84, wherein a non-tolerant T cell is a T cell from the recipient that is predicted to fail T cell selection in the donor.

86. The method of claim 84, wherein the non-tolerant T cell is a T cell from the recipient that is likely to drive an organ or cellular transplant rejection.

87. The method of claim 73, 74, 79, 82 or 83, wherein the sample is peripheral blood or a tissue sample.

88. A method of predicting a risk of developing graft-versus-host disease (GvHD) from organ or cellular transplant in a recipient comprising:
a) reconstituting T cell selection in a recipient by classifying each TCRβ gene as a productive TCRβ gene or a repaired TCRβ gene using the machine learning system of any one of claims 45-66,
b) applying the T cell selection reconstituted from the recipient to the donor, and
c) determining a number of T cells from the organ or cells that are non-tolerant to a recipient,
wherein a number of non-tolerant T cells in the donor higher than a threshold indicates a risk of having or of developing GvHD from organ or cellular transplant,
thereby predicting a risk of developing GvHD from organ or cellular transplant in the recipient.

89. The method of claim 88, wherein reconstituting T cell selection in the recipient comprises sequencing T cell receptors (TCRβ) genes in a sample from the recipient and classifying each TCRβ gene as a productive TCRβ gene or a repaired TCRβ gene.

90. The method of claim 88, wherein applying the T cell selection to the donor comprises sequencing TCRβ genes in a sample from the donor and classifying each TCRβ gene as a productive TCRβ gene or a repaired TCRβ gene.

91. The method of claim 88, wherein non-tolerant T cells are T cells with a productive TCR gene misclassified as a repaired TCRβ gene.

92. The method of claim 91, wherein a non-tolerant T cell is a T cell from the donor that is predicted to fail T cell selection in the recipient.

93. The method of claim 91, wherein the non-tolerant T cell is a T cell from the donor that is likely to drive GvHD.

94. The method of claim 89, wherein the sample from the recipient is peripheral blood or a tissue sample.

95. The method of claim 90, wherein the sample from the donor is a sample from the transplant.

96. A method of predicting a risk of developing alloimmunity from an adoptive T cell therapy in a recipient comprising:
a) reconstituting T cell selection in a recipient by classifying each TCRβ gene as a productive TCRβ gene or a repaired TCRβ gene using the machine learning system of any one of claims 45-66,
b) applying the T cell selection reconstituted from the recipient to the donor T cells, and
c) determining a number of T cells from the donor being donated that are non-tolerant to the recipient,
wherein a number of non-tolerant T cells in the donor higher than a threshold indicates a risk of having or of developing alloimmunity from an adoptive T cell therapy, thereby predicting a risk of developing alloimmunity from an adoptive T cell therapy in the recipient.

97. The method of claim 96, wherein reconstituting T cell selection in the recipient comprises sequencing T cell receptors (TCRβ) genes in a sample from the recipient and classifying each TCRβ gene as a productive TCR gene or a repaired TCRβ gene.

98. The method of claim 96, wherein applying the T cell selection from the recipient to the donor T cells comprises sequencing TCRβ genes in a sample from the recipient and classifying each TCRβ gene as a productive TCRβ gene or a repaired TCRβ gene.

99. The method of claim 96, wherein non-tolerant T cells are T cells with a productive TCR gene misclassified as a repaired TCRβ gene.

100. The method of claim 99, wherein a non-tolerant T cell is a T cell from the donor that is predicted to fail T cell selection in the recipient.

101. The method of claim 99, wherein the non-tolerant T cell is a T cell from the donor that is likely to drive alloimmunity in the recipient.

102. The method of claim 96, wherein alloimmunity from an adoptive T cell therapy comprises unwanted immune attacks from the donor T cells against the recipient's cells and tissues.

103. The method of claim 97 or 98, wherein the sample is peripheral blood or a tissue sample.

104. The method of claim 91, wherein adoptive T cells in the adoptive T cell therapy are allogenic CAR T cells.

105. The method of claim 91 wherein adoptive T cells in the adoptive T cell therapy are allogenic T cells with an engineered TCR.

106. A method of predicting compatibility of an engineered T cell receptor (TCR) therapy in a recipient comprising:
a) reconstituting T cell selection in a recipient by classifying each TCRβ gene as a productive TCRβ gene or a repaired TCRβ gene using the machine learning system of any one of claims 45-66,
b) applying the T cell selection reconstituted from the recipient to the engineered TCR, and
c) determining if the engineered TCR is non-tolerant to the recipient, thereby predicting compatibility to an engineered TCR therapy.

107. The method of claim 106, wherein reconstituting T cell selection in the recipient comprises sequencing T cell receptors (TCRβ) genes in a sample from the recipient and classifying each TCRβ gene as a productive TCRβ gene or a repaired TCRβ gene.

108. The method of claim 106, wherein applying the T cell selection from the recipient to the engineered TCR comprises sequencing TCRβ genes in a sample from the recipient and classifying each TCRβ gene as a productive TCRβ gene or a repaired TCR gene.

109. The method of claim 106, wherein a non-tolerant TCR is an engineered TCRβ gene misclassified as a repaired TCRβ gene.

110. The method of claim 106, wherein a non-tolerant TCR is an engineered TCR predicted to fail T cell selection in the recipient.

111. The method of claim 110, wherein the non-tolerant TCR is an engineered TCR that is likely to drive alloimmunity in the recipient.

112. The method of claim 106, wherein alloimmunity from an adoptive T cell therapy comprises unwanted immune attacks from the engineered TCR against the recipient's cells and tissues.

113. The method of claim 107 or 108, wherein the sample is peripheral blood or a tissue sample.

114. A method of predicting a risk of developing an autoimmune disease or disorder in a subject comprising:
a) reconstituting B cell selection in healthy subjects by classifying each B cell receptor (BCR) genes as a productive BCR gene or a repaired BCR gene using the machine learning system of claim 42, wherein the immune receptor chain gene is BCR gene,
b) applying the B cell selection reconstituted from the healthy donors to B cells from the subject, and
c) evaluating a number of escaped B cells in the subject that fail B cell selection in the healthy donor,
wherein a number of escaped B cells higher than a threshold indicates a risk of having or of developing an autoimmune disease or disorder,
thereby predicting a risk of developing an autoimmune disease or disorder in the subject.

115. The method of claim 114, wherein the gene segments are selected from the group consisting of variable (V) gene segments, diversity (D) gene segments, joining (J) gene segments and any combination thereof.

116. The method of claim 114, wherein the selection prediction identifies BCR gene as a productive BCR gene or a repaired BCR gene.

117. The method of claim 114, wherein the machine learning system includes an ensemble of multiple prediction models, each prediction model included in the ensemble of multiple prediction models generates a model prediction and the model predictions from each prediction model are combined to determine the selection prediction.

118. The method of claim 114, wherein a modified neural decision tree architecture including a hierarchical arrangement of more than two consecutive decisions is used to aggregate the model predictions into the selection prediction.

119. The method of claim 118, wherein the architecture of the neural decision tree includes a committee of functions, a number of functions included in the committee of functions increasing from the terminal decision in the neural decision tree to base decision on the neural decision tree.

120. The method of claim 114, further comprising obtaining a training dataset including a library of BCR genes and the BCR protein sequences of the BCR genes; and
training the one or more prediction models included in the machine learning system using the training dataset by determining the weight values included in each prediction model using an optimization process.

121. The method of claim 114, wherein the library of BCR genes includes multiple productive genes and multiple non-productive genes.

122. The method of claim 121, wherein a non-productive BCR gene is a BCR gene with out-of-frame gene segments or a BCR gene with a stop codon in a somatic junction between gene segments.

123. The method of claim 122, further comprising repairing each of the multiple non-productive genes; and translating each of the repaired non-productive genes into a BCR protein sequence.

124. The method of claim 123, wherein repairing non-productive BCR gene comprises adding or removing one or more nucleotides at a somatic junction between gene segments to bring the gene segments in a same reading frame and/or mutating a nucleotide in a somatic region between gene segments to convert a stop codon into an amino acid.

125. The method of claim 123, wherein repairing an BCR gene identified as non-productive comprises generating a repaired BCR gene.

126. The method of claim 114, wherein the library of BCR genes and BCR protein sequences are obtained from a sample provided by an HLA-matched healthy donor.

127. The method of claim 126, wherein the sample is peripheral blood or a tissue sample.

128. The method of claim 114, wherein the protein feature includes a piece of data related to a property of an amino acid, the property is at least one of a polarity, one or more secondary structure associations, a molecular volume, a codon diversity, or an electrostatic charge.

129. The method of claim 114, wherein each of the repaired non-productive genes is weighted according to a probability that a repair used to generate a particular repaired non-productive gene appears naturally among the subject's non-productive genes.

130. The method of claim 114, wherein reconstituting B cell selection in healthy subjects comprises sequencing B cell receptor (BCR) genes in a sample from the healthy subjects and classifying each BCR gene of the healthy subjects as a productive BCR gene or a repaired BCR gene.

131. The method of claim 114, wherein applying the B cell selection comprises sequencing BCR genes in a sample from the subject and classifying each TCR gene as a productive TCR gene or a repaired TCR gene.

132. The method of claim 114, wherein escaped B cells are B cells with a productive BCR gene misclassified as a repaired BCR gene.

133. A method of predicting an antibody drug safety in a subject comprising:
a) reconstituting B cell selection in the subject by classifying each B cell receptor (BCR) gene of the subject as a productive BCR gene or a repaired BCR gene using the machine learning system of claim 42, wherein the immune receptor chain gene is BCR gene, and
b) determining if a BCR gene encoding the antibody drug is tolerant to subject's self-antigens,
wherein a tolerant BCR gene encoding an antibody drug is a BCR gene correctly classified as a productive BCR gene,
thereby predicting an antibody drug safety in the subject.

134. The method of claim 133, wherein the gene segments are selected from the group consisting of variable (V) gene segments, diversity (D) gene segments, joining (J) gene segments, and any combination thereof.

135. The method of claim 133, wherein the selection prediction identifies BCR gene as a productive BCR gene or a repaired BCR gene.

136. The method of claim 133, wherein reconstituting B cell selection in the subject comprises sequencing BCR genes in a sample from the subject and classifying each BCR gene of the subject as a productive BCR gene or a repaired BCR gene.

137. The method of claim 133, wherein a non-tolerant BCR gene encoding an antibody drug is a BCR gene misclassified as a repaired BCR gene.

138. The method of claim 137, wherein a non-tolerant BCR gene encoding an antibody drug is a BCR gene that is predicted to fail B cell selection in the subject.

139. The method of claim 138, wherein the non-tolerant BCR gene encoding an antibody drug encodes an antibody drug that is likely to bind self-antigens in the subject.

140. The method of claim 139, wherein an antibody drug classified as likely to bind self-antigen indicates a lack of safety of use of the antibody drug in the subject.

141. The method of claim 133, wherein the sample is peripheral blood or a tissue sample.

142. A method of predicting a risk of developing alloimmunity from a chimeric antigen receptor (CAR)-T cell therapy in a subject comprising determining if an antigen binding domain of the CAR is tolerant to subject's self-antigens,
wherein determining if an antigen binding domain of the CAR is tolerant to subject's self-antigens comprises:
a) reconstituting B cell selection in the subject by classifying each BCR gene of the subject as a productive BCR gene or a repaired BCR gene using the machine learning system of claim 42, wherein the immune receptor chain gene is BCR gene, and
b) determining if a B cell receptor (BCR) gene encoding the antigen binding domain of the CAR is tolerant to subject's self-antigens,
wherein a tolerant BCR gene encoding the antigen binding domain of the CAR is a BCR gene correctly classified as a productive BCR gene,
thereby predicting a risk of developing alloimmunity from a chimeric antigen receptor (CAR)-T cell therapy in the subject.

143. The method of claim 142, wherein reconstituting B cell selection in the subject comprises sequencing BCR genes in a sample from the subject and classifying each BCR gene of the subject as a productive BCR gene or a repaired BCR gene.

144. The method of claim 142, wherein a non-tolerant BCR gene encoding the antigen binding domain of the CAR is a BCR gene misclassified as a repaired BCR gene.

145. The method of claim 142, wherein a non-tolerant BCR gene encoding the antigen binding domain of the CAR is a BCR gene that is predicted to fail B cell selection in the subject.

146. The method of claim 145, wherein the non-tolerant BCR gene encoding an antibody drug encodes an antibody drug that is likely to bind self-antigens in the subject.

147. The method of claim 146, wherein a BCR gene classified as likely to bind self-antigen indicates a lack of safety of use of the CAR-T cell therapy in the subject.

148. The method of claim 142, wherein the sample is peripheral blood or a tissue sample.

149. The method of claim 73, wherein the sample matching healthy donor is a biospecimen from the subject collected prior to the development of any symptom of a disease.

150. The method of claim 149, wherein the biospecimen is banked blood.

151. The method of claim 149, wherein the biospecimen is collected prior to an immune checkpoint inhibitor therapy.
